(19) United States
(12) Patent Application Publication
(10) Pub. No.: US 2020/0041493 A1
(43) Pub. Date: Feb. 6, 2020

(54) LEUKOCYTE ADHESIVE FUNCTION ASSAYS, DEVICES AND/OR USES

(71) Applicant: STICKYCELL PTY LTD, Lutwyche (AU)

(72) Inventor: Qiang Cheng, Lutwyche (AU)

(21) Appl. No.: 16/341,684

(22) PCT Filed: Oct. 16, 2017
(86) PCT No.: PCT/AU2017/051118

§ 371 Date: Apr. 12, 2019

(30) Foreign Application Priority Data
Oct. 14, 2016 (AU) 2016904169

(51) Int. Cl. G01N 33/50; G01N 33/58

(52) U.S. Cl. CPC G01N 33/5094; G01N 2800/52; G01N 33/581

(57) ABSTRACT

The present disclosure is directed relates to assays, including but not limited to, leukocyte adhesive function assays (LAFA), devices and/or methods of using such assays. Disclosed embodiments may be used in diagnostic, analytic and/or prognostic applications. Certain embodiments are also related to stratifying, predicting and/or determining how one or more subjects are likely to respond and/or is responding to a drug. The present disclosure also relates to one or more methods of optimising a dosage regimen for one or more subjects taking a drug. In addition, the present disclosure is also related to minimise or potentially reduce drug side effects.

### TECHNICAL FIELD

The present disclosure relates to assays, including but not limited to, leukocyte adhesive function assays (LAFA), devices and/or methods of using such assays. The present disclosure also relates to the uses of the disclosed embodiment in diagnostic, analytic and/or prognostic applications. The present disclosure also relates to assessing the abnormal activation leukocyte adhesion molecules and/or chemokine receptors. The present disclosure is also related to stratifying, predicting and/or determining how one or more subjects are likely to respond and/or is responding to a drug. The present disclosure also relates to one or more methods of optimising a dosage regimen for one or more subjects taking a drug. In addition, the present disclosure is also related to minimise or potentially reduce drug side effects.

### CROSS REFERENCE

This application claims priority to and is related to Australian Application No. 2016904169 entitled “Leukocyte Adhesive Function Assays, Devices and/or Uses” filed on 14 Oct. 2016. This application is incorporated herein by reference in its entirety. In addition, the other references or publications referred to in the present disclosure are also hereby incorporated by reference in their entirety.

### BACKGROUND

If a prior art publication is referred to herein, this reference does not constitute an admission that the publication forms part of the common general knowledge in the art in Australia or in any other country.

Leukocyte recruitment from the circulation to the surrounding tissue is one of the early but critical steps during the induction of inflammation. To be recruited from the fast traveling blood stream, leukocytes undergoing a sequence of interactions with blood vessel endothelium, including tethering, rolling, slow rolling, firm adhesion, crawling and eventually trans-endothelial migration. It understood that these leukocyte and endothelium interactions are dependent on the physical interactions between specific membrane molecules (such as adhesion molecules, chemokines and chemokine receptors), expressed by both leukocytes and endothelial cells.

First, circulating leukocytes tether and roll along the endothelial surface via the interaction between leukocyte-expressed PSGL-1 (P-selectin glycoprotein ligand-1) and its endothelial ligands, P-selectin and E-selectin. Rolling leukocytes subsequently reduce their rolling velocity as a result of chemokine induced cell activation. This allows the interaction between leukocyte β2 and α4 integrins with their endothelial ligands, including intercellular adhesion molecule-1 (ICAM-1) and vascular cell adhesion molecule-1 (VCAM-1), leading to leukocyte firm adhesion on endothelial surface. Adherent leukocytes are able to use αL integrin (CD11a) and αM integrin (CD11b) to interact with endothelial ICAM-1, allowing leukocytes to crawl on the endothelial surface before finding a site for leukocyte extravasation.

Under disease conditions, however, the functions of these adhesion molecules are altered, leading to abnormal leukocyte recruitment and inflammation. Numerous studies have demonstrated an abnormal appearance of leukocytes in tissue biopsy samples from patients with inflammatory diseases, which is correlated with irreversible organ damage during disease progression. For example, an increased α4β1 integrin expression and activity has been reported on leukocytes from patients with multiple sclerosis (MS), a chronic autoimmune disease characterised by demyelination and degeneration of axons in the central nervous system, as a result of increased leukocyte infiltration across the brain blood barrier (BBB). To attenuate the α4 integrin activation, an anti-human α4 integrin antibody. Natalizumab, has been developed.

Accordingly, Natalizumab therapy has been shown to reduce leukocyte infiltration across the brain blood barrier and, therefore, eliminate disease progression. Thus, identification of such abnormality in leukocyte adhesive functions provides information on their potential to leave circulation and their ability to cause tissue damage.

Despite its role in the disease pathogenesis, the ability to assess leukocyte adhesive function is missing from the current monitoring and testing regimes being used in clinical settings. In research laboratories, a parallel plate flow chamber technique may be used to study leukocyte and endothelial cell interactions. However, the existing techniques lack an ability to accurately assess leukocyte migration and/or leukocyte adhesive function under typical clinical settings and time limits. For example, to study a specific subset of leukocytes, the cells in a research laboratory usually need to be isolated from whole blood. The isolated leukocytes are then used for the flow chamber assay in the absence of other blood components (e.g. red blood cells and platelets), which are known to be key regulators of leukocyte recruitment. This isolation process not only requires a large volume of human blood, but also alters the activation status of the leukocytes, which may affect and/or compromise the following assay. Additionally, current imaging techniques lack the ability to quantitatively assess adhesive functions of specific adhesion molecules, limiting their applications in clinical and drug contexts.

There is a need in the art for solutions to one or more of these features and/or disadvantages. The present disclosure describes exemplary embodiments address one or more of the features and/or advantages disclosed herein. The present disclosure is directed to solving these and other problems disclosed herein. The present disclosure is also directed to overcoming and/or ameliorating at least one of the disadvantages of the prior art as will become apparent from the discussion herein.

### SUMMARY

Exemplary embodiments are to new uses for leukocyte adhesive function assays and devices.

Exemplary embodiments are to one or more leukocyte adhesive function assays for assessing leukocyte migration under realistic physiological conditions or under conditions that mimic, attempt to mimic or substantially mimic in vivo conditions.

Exemplary embodiments are to assays that measure the ability of leukocyte to adhere to endothelial cells, endothelial adhesion molecules, endothelial membrane proteins or combination thereof. For example, under realistic physiological conditions or under conditions that mimic, attempt to mimic or substantially mimic in vivo conditions.

Exemplary embodiments are to leukocyte adhesive function assays requiring only a small amount of whole blood, isolated leukocytes, cultured leukocytes and/or leukocyte cell lines.

Exemplary embodiments provide a method (or methods) to assess a subject's response, or potential response, to a drug treatment suitable for controlling progress of a disease, wherein the drug is capable of altering leukocyte recruitment, adhesion and/or migration, the method comprising the steps of:
obtaining a blood sample from the subject:
subjecting the blood sample to at least one leukocyte function assay (LAFA), wherein the LAFA assesses leukocyte recruitment, adhesion and/or migration to at least one or more of the following: at least one endothelial molecule and at least one cell; and
based at least in part on one or more results of the at least one LAFA, assess a subject's respond, or potential response, to the drug treatment for controlling progression of the disease.

Exemplary embodiments provide a method (or methods) to assess a subject's response, or potential response, to a drug treatment suitable for controlling progress of a disease, wherein the drug is capable of altering leukocyte recruitment, adhesion and/or migration, the method comprising the steps of:
obtaining a blood sample from the subject;
subjecting the blood sample to at least one leukocyte function assay (LAFA), wherein the at least one LAFA quantitatively and/or semi-quantitatively assesses leukocyte recruitment, adhesion and/or migration under realistic physiological conditions to at least one or more of the following: at least one endothelial molecule and at least one cell expressing an endothelial molecule; and
based at least in part on one or more results of the at least one LAFA, assess a subject's respond, or potential response, to the drug treatment for controlling progression of the disease.

Exemplary embodiments provide a method (or methods) to assess adhesive function of one or more leukocytes molecules, the method comprising the steps of:
obtaining a blood sample from a subject;
subjecting the blood sample to at least one leukocyte function assay (LAFA), wherein the at least one LAFA quantitatively and/or semi-quantitatively assesses leukocyte recruitment, adhesion and/or migration under realistic physiological conditions to at least one or more of the following: at least one endothelial molecule and at least one cell expressing an endothelial molecule; and
based at least in part on one or more results of the at least one LAFA, assess a level of activation of the one or more leukocytes molecules.

Exemplary embodiments provided methods for one or more of the following: (1) predicting how a subject is likely to respond to a drug for controlling progression of a disease, (2) determining whether a drug can be used to control and/or prevent progression of a disease in a subject, (3) choosing a drug for preventing and/or controlling progression of a disease in a subject, and (4) identifying a drug for preventing and/or controlling progression of a disease in a subject, wherein the drug is capable of at least altering leukocyte adhesion of one or more leukocyte cells to an endothelial molecule, said methods comprising the steps of: subjecting at least one blood sample obtained from the subject to at least one leukocyte adhesive function assay in vitro; and based on a result of the at least one assay, (a) predicting how the subject is likely to respond to the drug for controlling progression of the disease, (b) determining whether the drug may be used to control and/or prevent progression of the disease in the subject, (c) choosing a drug for preventing and/or controlling progression of the disease in the subject, and/or (d) identifying a drug for preventing and/or controlling progression of the disease in the subject.

Exemplary embodiments provided methods for one or more of the following: (1) predicting how one or more subjects is likely to respond to a drug for controlling progression of a disease, (2) determining whether a drug can be used to control and/or prevent progression of a disease in one or more subjects, (3) choosing one or more drug for preventing and/or controlling progression of a disease in one or more subjects, and (4) identifying one or more drugs for preventing and/or controlling progression of a disease and/or diseases in one or more subjects, wherein the one or more drugs is capable of at least altering leukocyte adhesion of one or more leukocyte cells to an endothelial molecule, said methods comprising the steps of:
subjecting at least one blood sample obtained from the one or more subjects to at least one leukocyte adhesive function assay in vitro; and
based on a result of the at least one assay, (a) predicting how the one or more subjects is likely to respond to the drug for controlling progression of the disease. (b) determining whether the one or more drugs may be used to control and/or prevent progression of the disease and/or diseases in the one or more subjects, (c) choosing the one or more drugs for preventing and/or controlling progression of the disease and/or diseases in the one or more subjects, and/or (d) identifying the one or more drugs for preventing and/or controlling progression of the disease and/or diseases in the one or more subjects.

Exemplary embodiments provided a method of (1) predicting how a subject is likely to respond to a drug for controlling progression of a disease, (2) determining whether a drug can be used to control and/or prevent progression of a disease in a subject, (3) choosing a drug for preventing or controlling progression of a disease in a subject, and/or (4) identifying a drug for preventing or controlling progression of a disease in a subject, wherein the drug is capable of altering leukocyte adhesion to an endothelial molecule, said method comprising the steps of:
subjecting at least one blood sample obtained from the subject to at least one leukocyte adhesive function assay in vitro; and
based on a result of the assay, (a) predicting how the subject is likely to respond to the drug for controlling progression of the disease, (b) determining whether the drug may be used to control and/or prevent progression of the disease in the subject, (c) choosing a drug for preventing and/or controlling progression of the disease in the subject, and/or (d) identifying a drug for preventing and/or controlling progression of the disease in the subject.

Exemplary embodiments are to a method of determining how a subject administered a drug for controlling progression of a disease is responding to that drug, wherein the drug is capable of altering leukocyte adhesion to an endothelial molecule, said method comprising the steps of:
subjecting at least one blood sample containing the drug obtained from the subject to at least one leukocyte adhesive function assay in vitro; and
based on a result of the assay, determining how the subject is responding to the drug.

Exemplary embodiments are to methods of determining how a subject administered one or more drug for controlling progression of a disease is responding to that one or more drug, wherein the one or more drug is capable of altering leukocyte adhesion to at least one endothelial molecule, said method comprising the steps of:
subjecting at least one blood sample containing the drug obtained from the subject to at least one leukocyte adhesive function assay in vitro; and
based on a result of the assay, determining how the subject is responding to the drug.

Exemplary embodiments, provide methods of optimising a dosage regimen for at least one subject taking one or more drugs for controlling progression of one or more diseases, wherein the one or more drugs is capable of altering at least in part leukocyte adhesion to one or more endothelial molecules, said method comprising the steps of:
subjecting at least one blood sample containing the drug obtained from the subject to at least one leukocyte adhesive function assay in vitro; and
based on a result of the assay, optimising the drug dosage regimen for the subject to control progression of the disease.

Exemplary embodiments, provide a method of optimising a dosage regimen for a subject taking a drug for controlling progression of a disease, wherein the drug is capable of altering leukocyte adhesion to an endothelial molecule, said method comprising the steps of:
subjecting at least one blood sample containing the drug obtained from the subject to at least one leukocyte adhesive function assay in vitro; and
based on a result of the assay, optimising the drug dosage regimen for the subject to control progression of the disease.

Exemplary embodiments provide methods of treating a patient with an effective drug dose and/or dose range and reducing side effects due to the administration the effective drug dose and/or dose range, wherein the drug is capable of altering at least in part leukocyte adhesion to one or more endothelial molecules, said method comprising the steps of:
(1) administering to the subject a known quantity of the drug for a period of time;
(2) after step (1), subjecting a blood sample obtained from the subject to a leukocyte adhesive function assay in vitro; and
(3) based at least in part on a result of the assay, repeating steps (1) and (2) until a minimum effective drug dose or drug does range for the subject may be determined.

Exemplary embodiments, provide a method of determining a minimum effective drug dose for a subject for controlling progression of a disease, wherein the drug is capable of altering leukocyte adhesion to an endothelial molecule, said method comprising the steps of:
(1) administering to the subject a known quantity of the drug for a predetermined period of time;
(2) after step (1), subjecting a blood sample containing the drug obtained from the subject to a leukocyte adhesive function assay in vitro; and
(3) based on a result of the assay, repeating steps (1) and (2) until a minimum effective drug dose for the subject can be determined for controlling progression of the disease.

Exemplary embodiments provide one or more flow assays or one or more flow devices for carrying out one or more of the methods as disclosed herein.

Exemplary embodiments provided methods of generating a leukocyte adhesion profile for at least one subject, said methods comprising the steps of:
subjecting at least one blood sample to at least one leukocyte adhesive function assay in vitro so as to assess the adhesion functions of different leukocyte subsets to one or more different endothelial molecules; and
using the assay result for one or more of the following: identifying one or more leukocyte abnormalities; determination of personalised pathogenesis; identification of one or more disease markers; identifying early signs of one or more diseases; disease prediction; disease prevention; assisting with early and/or accurate diagnosis; developing an effective and personalised treatment for the subject; monitoring the health status) of one or more subjects; grouping different subjects regardless of disease: developing a treatment for one or more subjects regardless of disease diagnosis; recommending a treatment prior to disease diagnosis; recommending a treatment with an unknown aetiology and/or without disease diagnosis; and recommending a treatment where the disease diagnosis is unknown.

Exemplary embodiments, provide a method of generating a leukocyte adhesion profile for a subject, said method comprising the steps of:
subjecting at least one blood sample obtained from the subject to at least one leukocyte adhesive function assay in vitro so as to quantitatively assess the adhesion functions of different leukocyte subsets to one or more different endothelial molecules at substantially the same time; and using the assay result for one or more of the following: identifying leukocyte abnormalities; determination of personalised pathogenesis; identification of new disease markers for diseases; identifying early signs of disease; disease prediction; disease prevention; assisting with early and accurate diagnosis; developing an effective and personalised treatment for the subject; monitoring the health (healthy status) of the subject; grouping different subjects regardless of disease; and developing a treatment for the subject regardless of disease diagnosis.

### BRIEF DESCRIPTION OF FIGURES

Embodiments of the present disclosure are described, by way of example only, with reference to the accompanying figures.

FIGS. 1A-I: Illustrates divergent effects of Mn²⁺ treatments on migration profiles of CD4, CD8 and CD15 cells on VCAM-1 substrate, according to exemplary embodiments. FIG. 1A illustrates CD4 (Green), CD8 (Red) and CD15 (Cyan) cells that were labelled with antibodies conjugated to different fluorophores in whole blood, allowing simultaneous detection of these leukocyte subsets. Cell recruitment on VCAM-1 substrate was assessed using microfluidic channels. In FIG. 1A, representative screenshots of control (left) and Mn²⁺ treated (right) blood samples are shown. FIG. 1B: Mn²⁺ shows the effects on the number of interacting CD4, CD8 and CD15 cells. Total interacting CD4 (FIG. 1C), CD8 (FIG. 1D) and CD15 (FIG. 1E) cells were divided into 4 groups according to their cell mean speed (Sₘₑₐₙ): S, static cells (Sₘₑₐₙ<5 μm/min); C, crawling cells (Sₘₑₐₙ=5-20 μm/min); SR, slow rolling cells (Sₘₑₐₙ=20-300 μm/min); and R, rolling cells (Sₘₑₐₙ=300-6000 m/min). Mn²⁺ effects on the number of cells in each group were determined. Furthermore, Mn²⁺ effects on the average speed (FIG. 1F), dwell time (FIG. 1G) and straightness (FIG. 1H) of interacting CD4, CD8 and CD15 cells were also assessed. In FIGS. 1B to 1H the data represents the mean±SEM of n=10 independent subjects per group. In FIG. 1I, an anti-human CD16-BV510 antibody was introduced to identify CD16⁺CD15⁺ double positive cells (neutrophils). As a result, the percentages of CD16⁺CD15⁺ cells in the total CD15⁺ cells were calculated in control and the Mn²⁺ treated samples and are illustrate in FIG. 1I. In FIG. 1I: (n=4), *, p<0.05; **, p<0.01.

FIGS. 2A-C: Illustrates Natalizumab inhibits CD4, CD8 and CD15 cell recruitment on VCAM-1 substrate, according to certain exemplary embodiments. Using the microfluidic channel system described in Example 1, blood was treated with various doses of Natalizumab before being analysed. Natalizumab effects on the recruitment of control (circle) and Mn²⁺ treated (square) CD4 (FIG. 2A), CD8 (FIG. 2B) and CD15 (FIG. 2C) cells were determined. The numbers above each curve indicate the number of interacting cells without Natalizumab treatment. Data represent mean±SEM of n=4-6 subjects per group.

FIGS. 3A-F: Shows the inhibitory effects of low (10 μg/ml) and high (300 μg/ml) dose of Natalizumab on leukocyte firm adhesion on TNFα activated HUVEC, according to certain exemplary embodiments. The effects of Natalizumab on leukocyte interaction with TNFα activated HUVEC were assessed using a parallel plate flow chamber system. Leukocytes were labelled with Hoechst33342 in whole blood before being used for the flow assay. The images were acquired at high frame rates (2 frames per second) for 5 min to capture all types of cell interactions. Whole blood was treated without or with low dose (10 μg/ml) of Natalizumab, and the number of interacting leukocytes and their average speed were determined and shown in FIG. 3A and FIG. 3B, respectively. In the same experiments, the effects of low dose Natalizumab on the portions of static, crawling, slow rolling and rolling cells were also assessed (FIG. 3C). Data represent mean±SEM of n=7 subjects per group. In separate experiments, high dose (300 μg/ml) Natalizumab effects on the number of interacting cells (FIG. 3D), the cell speed (FIG. 3E) and the four cell interaction types (FIG. 3F) were determined. Data represent mean±SEM of n=6 subjects per group. *, p<0.05; **, p<0.01.

FIGS. 4A-F: Illustrates low and high dose Natalizumab alter cell migratory behaviours of CD4 and CD15 cells, but not CD8 cells, on TNFα activated HUVEC. The effects of Natalizumab on migratory behaviours of slowing moving (static and crawling) leukocytes on TNFα activated HUVEC was assessed using a parallel plate flow chamber system, according to certain exemplary embodiments. CD4, CD8 and CD15 cells were labelled with antibodies conjugated to different fluorophores in unprocessed whole blood, allowing simultaneous detection of these leukocyte subsets. Data was recorded as 3D image stacks at low frame rates (1 stack per 30 seconds) for 30 min, allowing the capture of detailed 3D movement of slow moving cells. Human whole blood was treated without or with low dose (10 μg/ml) of Natalizumab before being used for the flow assay. The number of interacting CD4, CD8 and CD15 cells (FIG. 4A), as well as their straightness (FIG. 4B) and average speed (FIG. 4C) were determined. Data represent mean±SEM of n=5 blood donors/group. In separate experiments, whole blood was treated without or with high dose (300 μg/ml) of Natalizumab before the flow assays. Natalizumab effects on the number of interacting cells (FIG. 4D), straightness (FIG. 4E) and speed (FIG. 4F) were then assessed. Data represent mean f SEM of n=6 subjects per group. *, p<0.05; **, p<0.01.

FIGS. 5A-C: Shows common origin graphs show Mn²⁺ inhibits the motility of CD4 and CD8 cells, but not CD15 cells on VCAM-1 substrate. VCAM-1 induced leukocyte recruitment were studied using a microfluidic system, according to certain exemplary embodiments. Human blood was treated without or with 5 mM Mn²⁺ before the assays. Images were analysed and interacting cells were tracked. Common origin graphs for each leukocyte subset were obtained by normalizing detected tracks to the same coordinates of origin.

FIGS. 6A-C: shows common origin graphs show low and high dose Natalizumab inhibits cell motility of CD4 and CD15 cells, but not CD8 cells, on TNFα activated HUVEC. Cell migratory behaviours on TNFα activated HUVEC were studied using a flow chamber technique, according to certain exemplary embodiments. Human whole blood was treated without or with low (10 μg/ml) or high (300 μg/ml) dose of Natalizumab before being used for the flow assays. Common origin graphs were generated as described in FIG. 5.

FIG. 7: Shows point of care blood test flow chart, according to exemplary embodiments.

FIGS. 8A and 8B: Shows ligand occupancy assay to examine Natalizumab occupancy of α4 integrin on CD4 lymphocytes, according to certain exemplary embodiments. Cells were activated with or without 5 mM MnCl2 before being used for the ligand occupancy assay. Cells were treated with various doses of Natalizumab and the Natalizumab occupancy was detected using a PE conjugated anti-human IgG secondary antibody. The percentage of PE positive cell (A) and PE MFI of CD4 lymphocytes (B) was determined by FACS. Data represent mean±SEM of n=3-4 subjects per group.

FIGS. 9A-H: Shows the effects of Mn²⁺ treatments on migration profiles of CD4 and CD8 cells on MAdCAM-1 substrate, according to certain exemplary embodiments. Blood samples collected from healthy volunteers were treated with or without 5 mM Mn²⁺ at room temperature for approximately 5 minutes, before being used for the LAFA. A: Mn² effects on the number of interacting CD4, CD8, CD15 and CD19 cells. The average speed (B), straightness (C) and dwell time (D) of interacting CD4, CD8, CD15 and CD19 cells were also assessed. Based on their mean speed of migration, total interacting cells were divided into static (<5 μm/min), crawling (5-20 μm/min), slow rolling (20-300 μm/min) and rolling cells (300-6000 μm/min). The Mn effects on the number of each type of interacting cells were show in 9E (CD4), 9F (CD8), 9G (CD15) and 9H (CD19), respectively. Data represent mean±SEM of n=14 independent subjects per group.

FIGS. 10A-D: Illustrates Vedolizumab weakens the interaction between CD4 cell and MAdCAM-1 substrate, according to certain exemplary embodiments. Using the microfluidic channel system as in FIG. 1, blood was treated with various doses of Vedolizumab before being analysed. Vedolizumab effects on the number of interacting CD4 (A) and CD8 (C) cell, as well as the speed of CD4 (B) and CD8 (D) cells were determined, in the absence (Blue circles) and the presence (Red squares) of Mn²⁺. The numbers above each curve show the number of interacting cells and their speed without Vedolizumab treatments. Data represent mean±SEM of n=3-4 subjects per group.

FIG. 11: Illustrates ligand occupancy assay to examine Vedolizumab occupancy of α4β7 integrin on CD4 lymphocytes, according to certain exemplary embodiments. Cells were activated with or without 5 mM MnCl₂ before being used for the ligand occupancy assay. Cells were treated with various doses of Vedolizumab and the Vedolizumab occupancy was detected using a PE conjugated anti-human IgG secondary antibody. The percentage of PE positive CD4 cells was determined by FACS. Data represent mean±SEM of n=2 subjects per group.

FIG. 12: Illustrates Vedolizumab effects on leukocyte recruitment on VCAM-1 substrate, according to certain exemplary embodiments. Whole blood was treated with or without 10 and 100 μg/ml of Vedolizumab, before being use for the leukocyte adhesive function assay. The number of interacting CD4, CD8, CD15 and CD19 cells were then determined. Data represent mean±SEM of n=3 subjects per group.

FIG. 13: Illustrates Natalizumab effects on leukocyte recruitment on MAdCAM-1 substrate, according to certain exemplary embodiments. Whole blood was treated with or without 10 μg/ml of Natalizumab, before being use for the leukocyte adhesive function assay using MAdCAM-1 as substrate. The number of interacting CD4 and CD8 cells were then determined. Data represent mean±SEM of n=4 subjects per group.

FIGS. 14A-D: Shows the effects of Natalizumab and Vedolizumab on leukocyte recruitment on P selectin and E selectin substrates, according to certain exemplary embodiments. Blood was treated with either 10 μg/ml Natalizumab or Vedolizumab before being used for LAFA analysis, using P selectin and E selectin as substrates. The number of interacting cells (A), speed (B), dwell time (C) and straightness (D) were then determined, as described in Example 1. Data represent mean±SEM of n=4-6 independent subjects per group. *, p<0.05.

FIGS. 15A-C: Shows the assessment of leukocyte CXCR1 and CXCR4 functions. VCAM-1 were co-coated with either IL-8 or SDF1α on the microfluidic channels, before being used for LAFA analysis, according to certain exemplary embodiments. The number of interacting CD4, CD8 and CD15 cells (A), their average straightness (B) and dwell time (C) were then determined, as described in Example 1. Data represent mean±SEM of n=6-8 independent subjects per group. *, p<0.05.

FIGS. 16A-P: Shows divergent responses of CD4 and CD8 cells from individual IBD patients to Vedolizumab treatments, according to certain exemplary embodiments. Blood samples were collected from patients with active inflammatory bowel disease. Blood was treated with 0.1 g/ml of Vedolizumab, before being analysed by LAFA exemplary embodiments using MAdCAM-1 as adhesive substrate. The number of interacting CD4 (A) and CD8 (C) cells, and their speed (B and D) of IBD patient #1 were then determined as detailed in Example 10. The same parameters for IBD patient #2 (E-H), patient #3 (1-L) and patient #4 (M-P) were also determined.

FIGS. 17A-B: Shows the detection of drug efficacy in MS patients undergoing Natalizumab therapy, according to certain exemplary embodiments. Blood samples were collected at various time points (2, 4, 6, 10 weeks) post Natalizumab infusion, and then analysed by LAFA exemplary embodiments using VCAM-1 as adhesive substrate. The number of CD4 interacting cells (FIG. 17A) and their dwell time (FIG. 17B) were then determined as described in Example 1. Blood samples from patients on Copaxone therapy were included as a negative control group.

FIGS. 18A-J: Illustrates personal profile (leukocyte adhesion fingerprint) of α4β2 (ligand of VCAM-1) adhesive functions, according to certain exemplary embodiments. Six blood samples were collected from a single healthy blood donor at different time-points during a period of approximately three months. This blood donor was suffered from wisdom tooth pain in the day when blood test #5 was conducted (squared). Blood test #4 was performed 7 days before blood test #5. Blood was analysed by LAFA exemplary embodiments in the presence or absence of 5 mM of MnCl2 using VCAM-1 as substrate, as described in Examples 1 and 4. The number of interacting CD4 cells (A), cell speed (B), straightness (C), dwell time (D) and dwell time activation potential ratio (DTAPR) (E) were then determined. The same parameters for CD8 leukocytes (F to J) were also assessed.

FIGS. 19A-J: Illustrates personal profile of α4β7 (ligand of MAdCAM-1) adhesive functions, according to certain embodiments. Six blood samples were collected from a single healthy blood donor at different time-points during a period of approximately three months. This blood donor was suffered from wisdom tooth pain in the day when blood test #5 was conducted (squared). Blood test #4 was performed 7 days before blood test #5. Blood was analysed by LAFA exemplary embodiments in the presence or absence of 5 mM of MnCl₂ using MAdCAM-1 as substrate, as described in Examples 10 and 11. The number of interacting CD4 cells (A), cell speed (B), straightness (C), dwell time (D) and straightness activation potential ratio (STAPR) (E) were then determined. The same parameters for CD8 leukocytes (F to J) were also assessed.

FIGS. 20A-L: Illustrates personal profile of PSGL-1 (ligand of P-selectin) adhesive functions, according to certain exemplary embodiments. Four blood samples were collected from a single healthy blood donor at different time-points during a period of approximately six weeks. This blood donor was suffered from wisdom tooth pain in the day when blood test #5 was conducted (squared). Blood test #4 was performed 7 days before blood test #5. Blood was analysed by LAFA exemplary embodiments using P-selectin as substrate, as described in Examples 16. The number of interacting CD4 cells (FIG. 20A), cell speed (FIG. 20B), straightness (FIG. 20C) and dwell time were then determined. The same parameters for CD8 leukocytes (Figures E to H) and CD15 leukocytes (FIGS. 20I to L) were also assessed.

FIGS. 21A-F: Shows the assessment of basal inflammatory status of α4f 1 integrin and α4β7 integrin in patients with multiple sclerosis (MS) and/or inflammatory bowel disease (IBD), according to certain exemplary embodiments. Blood samples were collected from healthy controls, MS and IBD patients, and then analysed by LAFA exemplary embodiments using VCAM-1 and MAdCAM-1 as substrates, according to certain exemplary embodiments. The Relative Straightness Index (RSTI) (FIG. 21A), Relative Speed Index (RSI) (FIG. 21B) and Relative Dwell Time Index (RDTI) (FIG. 21C) on VCAM-1 substrate were calculated as detailed in Examples 3 and 11. Similarly, RSTI (FIG. 21D), RSI (FIG. 21E) and RDTI (FIG. 21F) on MAdCAM-1 were determined. Each dot on the graph presents one individual subject. *: p<0.05, **: p<0.01, related to correspondent healthy controls.

FIG. 22A-F: Illustrates the assessment of Mn²⁺-induced activation potential of α4β1 integrin and α4β7 integrin in patients with multiple sclerosis (MS) and inflammatory bowel diseases (IBD), according to certain exemplary embodiments. Blood samples were collected from healthy controls, MS and IBD patients, and then analysed by LAFA exemplary embodiments using VCAM-1 and MAdCAM-1 as substrates. Straightness Activation Potential Ratio (STAPR) (FIG. 22A), Speed Activation Potential Ratio (SAPR) (FIG. 22B) and Dwell Time Activation Potential Ratio (DTAPR) (FIG. 22C) on VCAM-1 substrates were calculated as detailed in Example 4. Similarly, STAPR (FIG. 22D), SAPR (FIG. 22E) and ATAPR (FIG. 22F) on MAdCAM-1 substrate were also determined. Each dot on the graph presents one individual subject. *: p<0.05, **: p<0.01, related to correspondent healthy controls.

FIG. 23: Shows Vedolizumab dose dependent inhibition of the recruitment of CD4 leukocytes from IBD patients on MAdCAM-1 substrate, according to certain exemplary embodiments. Blood samples were collected from IBD patients, and then treated with a range of doses of Vedolizumab before being used for LAFA exemplary embodiments using MAdCAM-1 as substrate. The Vedolizumab IC50 values were then determined in individual IBD patients, as detailed in Example 12.

FIGS. 24A-H: To assess the functions of leukocyte expressing CXCR1 and CXCR4 in patients with multiple sclerosis (MS) and/or inflammatory bowel diseases (IBD), according to certain exemplary embodiments. Blood was collected from healthy subjects (n=8), MS patients (n=2) and IBD patients (n=4), and then used for LAFA exemplary embodiments using VCAM-1+IL-8 or VCAM-1+SDF1α as substrates, as detailed in Example 17. The number of interacting CD4, CD8 and CD15 leukocytes (FIG. 24A), cell speed (FIG. 24B), straightness (FIG. 24C) and dwell time (Figure D) were then determined on VCAM-1+IL-8 substrates, as detailed in Example 1. Similarly, the parameters (Figures E-H) were also assessed on VCAM-1+SDF1α substrates. *, p<0.05, **, p<0.01.

FIGS. 25A-D: Illustrates the assessment of leukocyte PSGL-1 adhesive function in MS and/or IBD patients, according to certain exemplary embodiments. Blood samples were collected from healthy controls (n=6), MS patients (n=2) and IBD patients (n=4), and then analysed by LAFA exemplary embodiments using P and E selectin as substrates, as detailed in Example 16. The number of interacting cells (FIG. 25A), speed (FIG. 25B), straightness (FIG. 25C) and dwell time (FIG. 25D) were then determined as detailed in Example 16. *: p<0.05.

FIG. 26: Illustrates a model to reduce the risk of Natalizumab induced PML, according to certain exemplary embodiments. After drug infusion, Natalizumab saturation level will be gradually reduced below maximal efficacy to a point (e.g. 80%) where a full drug efficacy may still be maintained, this may be defined as Drug Redosing Window. The reduction of drug saturation to below maximal drug efficacy may lead to a reconstitution of leukocyte recruitment and/or immune response, which could allow the immune system to restore the ability to respond to and eliminate JCV infection, leading to a reduced risk of PML. This drug redosing window may be accurately identified in one or more subjects by LAFA exemplary embodiments.

FIG. 27: Illustrate a flow chart for Image and Data analysis. Images captured in leukocyte adhesive function assay (LAFA) were processed and analysed using Trackmate from Fiji image analysis software, according to certain exemplary embodiments. The outputs from Trackmate were further analysed by a R program to generate descriptive statistics. The uses of the 5 programmes involved in the image analysis process was also indicated.

### DETAILED DESCRIPTION

The present disclosure is described in further detail with reference to one or more embodiments, some examples of which are illustrated in the accompanying drawings. The examples and embodiments are provided by way of explanation and are not to be taken as limiting to the scope of the disclosure. Furthermore, features illustrated or described as part of one embodiment may be used by themselves to provide other embodiments and features illustrated or described as part of one embodiment may be used with one or more other embodiments to provide further embodiments. The present disclosure covers these variations and embodiments as well as other variations and/or modifications.

As mentioned, exemplary embodiments are to new uses for leukocyte adhesive function assays and/or devices.

A leukocyte adhesive function assay may be used to determine how one or more subjects react or may react to a drug and/or combinations of drugs. In certain exemplary embodiment's one of or more of the advantages discussed herein may be present when the leukocyte adhesive function assay is to determine how one or more subjects react or may react to a drug and/or combinations of drugs. One or more of these advantages may also be found in other uses of the leukocyte adhesive function assay exemplary embodiments disclosed in this application. An advantage of this is that the in vitro function assay may be used to predict the effects of the drug in vivo. An advantage of this is that drug non-responders may be differentiated from drug responders by quantitatively assessing the activation levels of the drug targets. Another advantage of this is that subjects may be treated on a personalised basis. That is, a drug dosage regimen may be optimised, or substantially optimised, for each subject—tailored to the individual subject's needs based at least in part on the result of the assay. Another advantage is that the subject need not be administered the drug more than that necessary to achieve a therapeutic effect. Yet another advantage is that unwanted side-effects caused by some drugs may be reduced by administering the minimum therapeutically effective amount and/or range. This advantage may be pertinent to those drugs that have pathological and/or life-threatening side-effects. By determining the minimum therapeutically effective amount and/or range for a subject, such drugs may possibly be more safely administered with less side effects. Another advantage of the leukocyte adhesive function assay is that it may provide a more accurate assessment of drug effectiveness, regardless of serum drug concentration. Another advantage of the leukocyte adhesive function assay is that it may provide a more accurate assessment of drug effectiveness and may not be dependent on serum drug concentration. Exemplary LAFA embodiments directly assess the functional effects of a drug on leukocytes, offering a more effective assessment of drug effectiveness. Different from conventional measurements of drug serum levels, certain LAFA embodiments provide a functional readout to indicate drug efficacy, which less subject to being interfered with by other factors (as compared to conventional approaches), including but not limited to drug serum concentrations and/or anti-drug antibody. For example, the production of anti-drug antibody may significantly reduce drug activity for certain drugs, which cannot be detected by simply measuring drug blood serum levels. On the other hand, LAFA exemplary embodiments may easily detect these effects from anti-drug antibody, showing an accuracy and/or sensitivity advantage of LAFA exemplary embodiments comparing to other conventional approaches.

For example, in certain exemplary embodiments a leukocyte adhesive function assay may be used to for one or more of the following: (a) predict how a subject is likely to respond to a drug for controlling progression of a disease, (b) determine whether a drug may be used to control or prevent progression of a disease in a subject: (c) choose a drug for preventing or controlling progression of a disease in a subject, and (d) identify a drug for preventing or controlling progression of a disease in a subject.

In certain exemplary embodiments, there is provided a method of (a) predicting how a subject is likely to respond to a drug for controlling progression of a disease, (b) determining whether a drug may be used to control or prevent progression of a disease in a subject. (c) choosing a drug for preventing or controlling progression of a disease in a subject, or (d) identifying a drug for preventing or controlling progression of a disease in a subject wherein the drug is capable of altering leukocyte adhesion to an endothelial molecule, said method comprising the steps of:
subjecting at least one blood sample obtained from the subject to at least one leukocyte adhesive function assay in vitro; and
based on a result of the assay, (a) predicting how the at least one subject is likely to respond to the drug for controlling progression of the disease, (b) determining whether the drug may be used to control or prevent progression of the disease in the subject. (c) choosing a drug for preventing or controlling progression of the disease in the subject, or (d) identifying a drug for preventing or controlling progression of the disease in the subject.

The method (or methods) may be used for personalised medicine.

The method (or methods) may be used for distinguishing a drug responder from a drug non-responder.

The method (or methods) may be used for testing many subjects, for subject stratification (patient grouping).

The method (or methods) may comprise the step of, in accordance with the assay result, trialling the drug on the subject for controlling progression of the disease.

The method (or methods) may comprise the step of, in accordance with the assay result, treating the subject with the drug for controlling progression of the disease.

The method (or methods) may comprise the step of, in accordance with the assay result, determining an effective minimum therapeutic dose of the drug for the subject for controlling progression of the disease whilst minimising unwanted side effects caused by the drug. The drug dose may allow for restoration of minimal leukocyte cell interaction with endothelial cells in vivo so as to minimise or prevent pathologies such as progressive multifocal leukoencephalopathy (PML). That is, the drug may be an immune-suppressive drug and the minimal therapeutic drug dose may allow minimal restoration of immune response, while maintaining sufficient drug efficacy in vivo so as to minimise the risk of, for example, PML.

The method (or methods) may comprise the step of, in accordance with the assay result, optimising a dosage regimen for the drug for the subject for controlling progression of the disease, for example, by altering drug dosage or changing the length of time between sequential drug administrations.

The method (or methods) may be used for predicting or determining whether a drug (i.e., compound, chemical, molecule, reagent, biologic, antibody or other) may be useful for controlling progression of a disease for which the drug has not previously been indicated.

The assay or assays of the method (or methods) may comprise the step of identifying an adhesion anomaly or abnormality or a drug target and then choosing an appropriate drug for controlling progression of the disease based on the drug target.

The assay or assays of the method (or methods) may comprise the step of identifying an adhesion anomaly or abnormality or drug target and then choosing an appropriate drug for controlling progression of the disease based on a reference database of drug targets and drugs for those targets. In this way, the disease itself need not actually be diagnosed and/or identified and/or known.

The assay or assays of the method (or methods) may comprise the step of building a database of drug targets and drugs. The database may be built based on known drug targets and drugs. The database may be built based on in vivo drug treatments.

The assay of the method may comprise the step of assaying for more than one adhesion anomaly or abnormality or drug target at the one time (e.g. 2, 3, 4, 5, 6, 7, 8, 9 or 10 drug targets or more).

The assay or assays of the method (or methods) may comprise the step of assaying for one or more of the following: one or more adhesion anomalies, one or more abnormalities, and one or more drug targets. For example, at least 2, 3, 4, 5, 6, 7, 8, 9 or 10 of the one or more adhesion anomalies, one or more abnormalities and/or one or more drug targets.

The method (or methods) may be a high throughput assay, testing for a plurality of adhesion anomalies or abnormalities or drug targets at the one time.

The method (or methods) may be used to generate a leukocyte adhesion fingerprint for a subject (personal profile for leukocyte adhesive functions).

The method (or methods) may be used to identify different leukocyte anomalies or abnormalities in a subject.

The method (or methods) may be used for identifying disease markers.

The method (or methods) may be used to group individuals/subjects (regardless of disease).

The method (or methods) may be used to develop a treatment for a subject regardless of disease diagnosis.

The method (or methods) may be used for high throughput drug screening in vivo or in vitro.

The method (or methods) may be used for industry scale drug screening in laboratory animals.

For example, in exemplary embodiments a leukocyte adhesive function assay may be used to determine how a subject administered a drug for controlling progression of a disease is responding to that drug.

In certain exemplary embodiments, there is provided a method of determining how a subject administered a drug for controlling progression of a disease is responding to that drug, wherein the drug is capable of altering leukocyte adhesion to an endothelial molecule, said method comprising the steps of:
subjecting at least one blood sample containing the drug obtained from the subject to at least one leukocyte adhesive function assay in vitro, and
based on a result of the assay, determining how the subject is responding to the drug.

The method (or methods) may be used for personalised medicine.

The method (or methods) may be used for distinguishing a drug responder from a drug non-responder.

The method (or methods) may be used for testing many subjects, for subject stratification (patient grouping).

The method (or methods) may be used for high throughput drug screening in vivo or in vitro.

The method (or methods) may be used for industry scale drug screening in laboratory animals.

The method (or methods) may comprise the step of, in accordance with the assay result, determining an effective minimum therapeutic dose of the drug for the subject for controlling progression of the disease whilst minimising unwanted side effects caused by the drug. The drug dose may allow for restoration of minimal leukocyte cell interaction with endothelial cells in vivo so as to minimise or prevent pathologies such as progressive multifocal leukoencephalopathy (PML).

The method (or methods) may comprise the step of, in accordance with the assay result, optimising a dosage regimen for the drug for the subject for controlling progression of the disease, for example, by altering drug dosage or changing the length of time between sequential drug administrations.

For example, in certain exemplary embodiments, a leukocyte adhesive function assay may be used to optimise a dosage regimen for a subject taking a drug for controlling progression of a disease.

In certain exemplary embodiments, there is provided a method of optimising a dosage regimen for a subject taking a drug for controlling progression of a disease, wherein the drug is capable of altering leukocyte adhesion to an endothelial molecule, said method comprising the steps of:
subjecting at least one blood sample containing the drug obtained from the subject to at least one leukocyte adhesive function assay in vitro; and
based on a result of the assay, optimising the drug dosage regimen for the subject to control progression of the disease.

The method (or methods) may comprise the step of, in accordance with the assay result, determining an effective minimum therapeutic dose of the drug for the subject for controlling progression of the disease whilst minimising unwanted side effects caused by the drug. The drug dose may allow for restoration of minimal leukocyte cell interaction with endothelial cells in vivo so as to minimise or prevent pathologies such as progressive multifocal leukoencephalopathy (PML). That is, the drug may be an immune-suppressive drug and the minimal therapeutic drug dose may allow minimal restoration of immune response, while maintaining sufficient drug efficacy in viro so as to minimise the risk of, for example, PML.

The method (or methods) may comprise the step of, in accordance with the assay result, optimising a dosage regimen for the drug for the subject for controlling progression of the disease, for example, by altering drug dosage or changing the length of time between sequential drug administrations. Each time the assay is carried out, the dosage regimen or minimum effective drug dose may be optimised accordingly.

The method (or methods) may provide an accurate assessment of drug effectiveness, regardless of serum drug concentration.

For example, in certain exemplary embodiments other embodiments a leukocyte adhesive function assay may be used to determine a minimum effective drug dose for a subject for controlling progression of a disease.

In certain exemplary embodiments, there is provided a method of determining a minimum effective drug dose for a subject for controlling progression of a disease, wherein the drug is capable of altering leukocyte adhesion to an endothelial molecule, said method comprising the steps of:
(1) administering to the subject a known amount of the drug for a predetermined period of time;
(2) after step (1), subjecting a blood sample containing the drug obtained from the subject to a leukocyte adhesive function assay in vitro; and
(3) based on a result of the assay, repeating steps (1) and (2) until a minimum effective drug dose for the subject may be determined for controlling progression of the disease.

The method (or methods) may comprise minimising unwanted side effects caused by the drug.

The method (or methods) may comprise the step of, in accordance with the assay result, optimising a dosage regimen for the drug for the subject for controlling progression of the disease (as described previously).

Drug sensitivity may be tested in the subject using IC50 or IC99 to obtain the minimum effective drug dose.

The minimum effective drug dose may allow for restoration of minimal leukocyte cell interaction with endothelial cells in vivo so as to minimise or prevent pathologies such as progressive multifocal leukoencephalopathy (PML). That is, the drug may be an immune-suppressive drug and the minimal therapeutic drug dose may allow minimal restoration of immune response, while maintaining sufficient drug efficacy in vivo so as to minimise the risk of, for example, PML.

In certain exemplary embodiments, there is provided a flow assay or flow device for carrying out the method as described in other exemplary embodiments disclosed herein.

In some embodiments, the method may entail performing a leukocyte adhesive function assay to identify a leukocyte adhesion abnormality, choosing a suitable drug based on the nature of the leukocyte adhesion abnormality, and determining the effect of the drug on the leukocyte adhesion abnormality.

This may entail carrying out the following steps: I. Subjecting a blood sample obtained from the subject to leukocyte adhesive function assay to identify a leukocyte abnormality; 2. Choosing a suitable drug candidate (or more than one drug candidate) that could potentially be used for such an abnormality 3. Treating a blood sample with the suitable drug candidate in vitro at various doses; 4. Performing a further leukocyte adhesive function assay to test the effect of the drug candidate on the leukocyte abnormality; 5. Choosing the best or most effective drug for the subject: 6. Administering the drug to the subject: 7. Taking blood from the subject at various time points post-drug administration, and, 8. Performing a leukocyte adhesive function assay to confirm the drug effect in the subject.

Determining the effect of a drug in a human subject may entail carrying out the following steps: 1. Collecting blood from the subject; 2. Performing a first leukocyte adhesive function assay to obtain a baseline; 3. Administering a drug to the subject; and, 4. Performing a leukocyte adhesive function assay at various time points post-drug administration to determine the drug effect.

Determining the effect of a drug in an animal model/laboratory animal subject (mouse, primate etc) may entail carrying out the following steps: 1. Collecting blood from the subject; 2. Performing a first leukocyte adhesive function assay to obtain a baseline; 3. Administering a drug to the subject at various doses (each dose will be an independent assay); and 4. Performing a leukocyte adhesive function assay at various time points post-drug administration to determine the drug effect.

Alternatively, an in vitro model may be used. This may entail carrying out the following steps: 1. Collecting blood from a subject: 2. Treating the blood with the drug at various doses; and, 3. Performing a leukocyte adhesive function assay at various time points after the start of drug treatment, to determine the drug effects and time required to reach such effects. This may be done in high throughput manner.

Drug

It is to be understood that the term “drug” as used herein includes a compound, chemical, molecule, reagent, biologic, antibody, other moiety and combinations thereof that has a physiological effect on the subject, regardless of whether the drug function is known or unknown. Suitable types of drugs that may be utilised in the methods described herein provided that the drug is capable of altering leukocyte adhesion to the endothelium molecule—regardless of whether drug function is known or unknown. In certain exemplary embodiments, the drug may be an inhibitor or promoter (agonist) of leukocyte adhesion. In certain exemplary embodiments, the drug is an inhibitor of leukocyte adhesion. For example, a drug may be developed for a purpose that is not related to leukocyte adhesive functions. Once administrated by a subject or subjects, however, this drug may have multiple effects on this subject. Thus, by analysing blood samples from this subject after drug administration, the potential effects of this drug on leukocyte adhesive functions may be determined. In addition, the drug effects on leukocyte adhesive functions may also be projected by in vitro treatments of this drug with blood samples before being analysed by LAFA exemplary embodiments. Thus, LAFA exemplary embodiments offer a tool to identify the unknown and/or off-target and/or side effects of drugs and/or compounds on leukocyte adhesive functions.

In some embodiments the drug may directly interfere with the binding of the leukocyte with the endothelial molecule. In some embodiments the drug may indirectly interfere with the binding of the leukocyte with the endothelial molecule. In some embodiments the drug may target, bind to, associate with or otherwise interfere with a leukocyte adhesion molecule or other binding molecule of the leukocyte. In some embodiments, the drug may target, bind to, associate with or otherwise interfere with the endothelial molecule. In some embodiments, the drug may target, bind to, associate with or otherwise interfere with both a leukocyte adhesive molecule or other binding molecule and endothelial molecule. In yet other embodiments, the drug may indirectly influence the adhesion/interaction between the leukocyte and endothelial molecule by way of exerting its effect upon another part or molecule of the leukocyte adhesion pathway. In yet other embodiments, the drug may: regulate expression of a gene that affects leukocyte adhesion (for example the drug may act on intracellular signalling pathways to regulate the expression of a gene that affects leukocyte adhesion): affect post-translational modification of a gene product (RNA or protein) that affects leukocyte adhesion; regulate transportation or translocation of a gene product that affects leukocyte adhesion, and/or regulate the release from intracellular storage of a gene product that affects leukocyte adhesion.

Suitable types of leukocytes include, but are not limited to, one or more of the following: neutrophils, eosinophils, basophils, CD4 T lymphocytes, CD8 T lymphocytes, T regulatory cells, B lymphocytes, dendritic cells, monocytes and natural killer cells.

Suitable types of leukocyte adhesion molecules or other binding molecules of the leukocyte include one or more of the following: selectins, integrins, chemokines, chemokine receptors and others types of molecules.

Suitable types of endothelial molecules include one or more of the following: selectins, cell adhesion molecules (CAMs), chemokines, chemokine receptors and other types of molecules.

Suitable types of leukocyte adhesion molecules include, but are not limited to one or more of the following: PSGL-1, L-selectin, α1 integrin, α2 integrin, α3 integrin, α4 integrin, α5 integrin, α6 integrin, α7 integrin, α8 integrin, α9 integrin, α10 integrin, α11 integrin, αD integrin αE integrin, αV integrin, αX integrin, CD11a (αL integrin), CD11b (αM integrin), β1 integrin, β2 integrin, β4 integrin, β5 integrin, β6 integrin, β7 integrin β8 integrin, CD44, ESL-1, CD43, CD66, CD15s and ALCAM.

Suitable types of endothelial molecules include one or more of the following: E-selectin, P-selectin, VCAM-1, ICAM-1, ICAM-2, MadCAM-1, PECAM, GlyCAM-1, JAM-A, JAM-B, JAM-C, JAM-4, JAM-L, CD34, CD99, VAP-1, L-VAP-2, ESAM, E-LAM, cadherins, and hyaluronic acid.

Suitable types of chemokines and chemokine receptors include one or more of the following: chemokines CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL26, CX3CL1, XCL1 and XCL2; chemokine receptors CXCR1, CXCR2, CXCR3, CXCR4, CXCR5, CXCR6, CCR1, CCR2, CCR3, CCR4, CCR5, CCR6, CCR7, CCR8, CCR9, CCR10, CCR11, CX3CR1 and XCR1.

In some embodiments the drug may regulate the activity of a cytokine or chemokine.

In some embodiments, the drug may alter post-translational modification of an adhesion molecule or chemokine, alter protein membrane translocation of an adhesion molecule, regulate the release of an adhesion molecule from intracellular storage, act on intracellular signalling pathways to regulate the expression of an adhesion molecule or chemokine gene, or regulate mobilisation of an adhesion molecule.

In some embodiments, the drug attenuates leukocyte α4 integrin activation.

In some embodiments, the drug interferes with the interaction between leukocyte α4 integrin and its endothelial molecule. Examples of leukocyte α4 integrin are α4β7 integrin, CD11a (αL integrin) and CD11b (αM integrin).

In some embodiments, the drug interferes with the interaction between leukocyte-expressed PSGL-1 (P-selectin glycoprotein ligand-1) and its endothelial molecule, being P-selectin and/or E-selectin.

In some embodiments, the drug interferes with the interaction between leukocyte β2 integrin and its endothelial molecule.

In some embodiments, the drug interferes with the interaction between intercellular adhesion molecule-1 (ICAM-1) and/or vascular cell adhesion molecule-1 (VCAM-1) and its/their leukocyte adhesion molecule.

Examples of drugs include antibodies that target a specific leukocyte adhesion molecule and/or binding molecule and/or endothelial molecule. For example, an anti-human antibody that targets a specific leukocyte adhesion molecule and/or binding molecule and/or endothelial molecule.

In certain embodiments, the drug is an antibody that interferes with the binding between α4 integrin and its endothelial molecule. The drug may be an anti-human α4 integrin antibody. In certain embodiments, the drug is Natalizumab.

In certain embodiments the drug is an antibody that interferes with the binding between α417 integrin and MAdCAM-1. The drug may be Vedolizumab.

In embodiments the drug is an antibody that interferes with the binding between CD11a (αL) and ICAM-1. The drug may be Efalizumab or Odulimomab.

In certain embodiments the drug is an antibody that interferes with the binding between CD11b (αM) and ICAM-1. The drug may be UK279, 276.

In certain embodiments the drug is an antibody that interferes with the binding between 12 integrin and its endothelial molecule. The drug may be Erlizumab or Roverlizumab.

In certain embodiments the drug is an antibody that interferes with the binding between 17 integrin its endothelial molecule. The drug may be Etrolizumab.

Yet other examples of suitable drugs include steroids such as glucocorticoids (corticosteroids). Suitable steroids include, but are not limited to, Budesonide, Cortisone, Dexamethasone, Methylprednisolone. Prednisolone, Prednisone and/or combinations thereof.

Yet other examples of suitable drugs include non-steroidal anti-inflammatory drugs (NSAIDs). Suitable NSAIDs include, but are not limited to, Celecoxib, Etoricoxib, Ibuprofen, Ketoprofen, Naproxen, Sulindac and/or combinations thereof.

Yet other examples of suitable drugs include immune selective anti-inflammatory derivatives (ImSAIDs). Suitable ImSAIDs include, but are not limited to, Sub-mandibular gland peptide-T (SGP-T), and Phenylalanine-glutamine-glycine (FEG) and/or combinations thereof.

Yet other examples of suitable drugs include bioactive compounds from plants (including herbs). Suitable compounds include, but arc not limited to, Plumbagin (from Plumbago zylanica) and Plumericin (from Himatanthus sucuuba) and/or combinations thereof.

Yet other examples of suitable drugs include those (including related metabolites) that may enter into the circulation to affect leukocyte biology and functions.

Disease

In the methods described herein the drug may be used to control progression of certain suitable disease or diseases. In some embodiments the drug is used to control a disease involving abnormal leukocyte recruitment. In some embodiments the drug is used to control a disease involving inflammation. In some embodiments the drug may be used to control progression of an autoimmune disease. In some embodiments the drug may be used to control progression of an immune-deficient disease. In some embodiments the drug may be used to control progression of an infectious disease. In patients with infectious diseases, the immune system is highly activated due to the invasion of foreign pathogens, leading to an elevated inflammation and increased leukocyte adhesive functions. After the pathogen being eliminated, the activated immune system may still maintain its high level of activity, resulting in unnecessary damages to tissues. To address this issue, anti-adhesion therapies may be used to attenuate the activated leukocytes to reduce tissue damage. In certain exemplary embodiments, the drug may be used for one or more of the following: to control at least in part progression of certain diseases: to control at least in part a disease involving abnormal leukocyte recruitment; to control a disease at least in part involving inflammation; to control progression at least in part of an autoimmune disease; to control progression at least in part of an immune-deficient disease; and to control at least in part progression of an infectious disease.

Diseases of interest include but are not limited to:

Inflammatory arthritis—e.g., rheumatoid arthritis, seronegative spondeloarthritites (Behcets disease, Reiter's syndrome, etc.), juvenile rheumatoid arthritis, vasculitis, psoriatic arthritis, polydermatomyositis, or combinations thereof.

Inflammatory dermatoses—e.g., psoriasis, dermatitis herpetiformis, eczema, necrotizing and cutaneous vasculitis, bullous diseases, or combinations thereof.

Systemic lupus erythematosus (SLE), asthma, reperfusion injury, septic shock (Sepsis), adult respiratory distress syndrome (ARDS), tissue damage relating to tissue transplantation, cardiopulmonary bypass, thermal injury (burn), including shock, pulmonary edema, or combinations thereof.

Other autoimmune disorders such as glomerulonephritis, juvenile onset diabetes, multiple sclerosis, allergic conditions, autoimmune thyroiditis, allograft rejection (e.g., rejection of transplanted kidney, heart, or liver), Crohn's disease, graft-versus-host disease, or combinations thereof.

Systemic inflammation, associated with the use of pump-oxygenator systems in cardiopulmonary bypass and hemodialysis, which may lead to organ dysfunction, termed the post-pump syndrome or post-perfusion syndrome, diabetes, or combinations thereof.

Other diseases and clinical correlates of undesirable inflammatory responses including those associated with hemolytic anemia, hemodialysis, blood transfusion, certain hematologic malignancies, pneumonia, post-ischemic myocardial inflammation and necrosis, barotrauma (decompression sickness), ulcerative colitis, inflammatory bowel disease, atherosclerosis, cytokine-induced toxicity, necrotising enterocolitis, granulocyte-transfusion-associated syndromes, Reynaud's syndrome, multiple organ injury syndromes secondary to septicemia or trauma, acute purulent meningitis, other central nervous system inflammatory disorders, or combinations thereof.

Other diseases that affect white blood cells, may include but not limited to: Lymphoma, Leukemia, Multiple myeloma, Myelodysplastic syndrome, Eosinophilia. Hodgkin lymphoma, or combinations thereof.

Autoimmune diseases may include but are not limited to: Addison's disease, Agammaglobulinemia, Alopecia areata, Amyloidosis, Ankylosing spondylitis. Anti-GBM/Anti-TBM nephritis, Antiphospholipid syndrome (APS), Autoimmune hepatitis, Autoimmune inner ear disease (AIED), Axonal & neuronal neuropathy (AMAN), Behcet's disease, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss, Cicatricial pemphigoid/benign mucosal pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia. Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hypogammalglobulinemia, IgA Nephropathy, IgG4-related sclcerosing disease, Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytolastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multiple sclerosis (MS), Myasthenia gravis, Myositis, Narcolepsy, Neuromyelitis optica, Neutropenia, Ocular cicatricial pemphigoid, Optic neuritis, Palindromic rheumatism (PR), PANDAS (Pediatric Autoimmune Neuropsychiatric Disorders Associated with Streptococcus), Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome (polyneuropathy, organomegaly, endocrinopathy, monoclonal gammopathy, skin changes), Polyarteritis nodosa. Polymyalgia rheumatica, Polymyositis, Postmyocardial infarction syndrome, Postpericardiotomy syndrome, Primary biliary cirrhosis. Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Reiter's syndrome, Relapsing polychondritis, Restless legs syndrome (RLS), Retroperitoneal fibrosis, Rheumatic fever, Rheumatoid arthritis (RA), Sarcoidosis, Schmidt syndrome, Scleritis, Scleroderma. Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis. Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis. Vitiligo, Wegener's granulomatosis (now termed Granulomatosis with Polyangiitis (GPA), or combinations thereof.

Diseases or disorders due to immunosuppression (e.g., due to AIDS, maycer chemotherapy, radiation therapy, corticosteroid therapy, or other therapy for autoimmune disease), congenital immunodeficiencies, or combinations thereof.

Infectious diseases, include but not limited to: ICAM-1 mediated infections such as rhinoviral infection, Amoebic meningoencephalitis, Acute rheumatic fever. Anthrax, atypical mycobacterial disease, Avian influenza (Bird Flu), Babesiosis, Bacterial vaginosis, Balanitis, Barmah Forest virus infection, Blastocystis infection, Botulism, Brucella infection, Campylobacter infection, Chickenpox and shingles, Chikungunya virus, Cold sores (herpes simplex type 1), Common cold, Conjunctivitis, Cryptosporidium infection, Cytomegalovirus (CMV) infection, Dengue fever, Giardia infection, Glandular fever, Gonorrhoea, Haemophilus influenzae type b (Hib), Hepatitis, Hand, foot and mouth disease, Hendra virus infection, Hydatid disease, Human papilloma virus (HPV), genital warts & related cancers, Japanese encephalitis, Kunjin/West Nile virus infection, Kunjin/West Nile virus infection, Leprosy, Legionella pneumophila infection, Leptospirosis, Listeria infection. Lyme disease, Measles, Meningococcal infection, Molluscum contagiosum. Mumps, Mycoplasma genitalium infection, Mycoplasma pneumoniae infection, Middle East respiratory syndrome (MERS), Non-specific urethritis (NSU), Norovirus infection, Parvovirus B19 infection, Plague, Pneumococcal infection, Poliovirus infection, Psittacosis, Q fever, Rabies virus and Australian bat lyssavirus, Respiratory syncytial virus (RSV) infection, Rickettsial infections, Roseola, Ross River virus infection, Rotavirus infection, Rubella, Salmonella infection, School sores, Severe acute respiratory syndrome, Shiga toxin producing Escherichia coli (STEC) and haemolytic uraemic syndrome (HUS), Shigella infection, Smallpox, Staphylococcus aureus including methicillin-resistant Staphylococcus aureus (MRSA), Streptococcal sore throat, Syphilis, Tetanus, Thrush, Toxic shock syndrome, Toxoplasma infection, Trichomonas infection, Tuberculosis, Tularaemia, Typhoid and paratyphoid, Urinary tract infection, Vibrio parahaemolyticus infection, Viral gastroenteritis, Viral haemorrhagic fevers, Viral meningitis, Viral respiratory infections, Warts, Whooping cough, Worms, Yellow fever, Yersinia infection, Yersinia infection, Zika virus infection, or combinations thereof

Diseases caused by red blood cell disorders, may include but not limited to: Anemia, Anemia of chronic disease, Aplastic anemia, Autoimmune hemolytic anemia. Thalassemia, Malaria, Sickle cell anemia. Polycythemia vera, Acute chest syndrome, Bahima disease, Erythroid dysplasia. Haemochromatosis type 3, Hemoglobin Lepore syndrome, Hemoglobin variants, Hemoglobinemia, Hemosiderinuria, Hereditary pyropoikilocytosis, HFE, Methemoglobinemia hereditary haemochromatosis, McLeod syndrome, Microcytosis, Myomatous erythrocytosis syndrome, Poikilocytosis, Polychromasia. Polycythemia, Porphyria, Reticulocytopenia, Rh deficiency syndrome, Sick cell syndrome, Spherocytosis, Sulfhemoglobinemia, Transient erythroblastopenia of childhood, or combinations thereof.

Diseases caused by platelet disorders, may include but not limited to: Thrombocytopenia, thrombocytopenia. Certain genetic disorders, Atrial fibrillation. Hemophilia, von Willebrand disease, epistaxis, menorrhagia, petechiae, telangiectasias, ecchymoses, Post-Transfusion Purpura, Cyclic (cyclical) Thrombocytopenia, Disseminated Intravascular Coagulopathy, Thrombotic Thrombocytopenic Purpura, Henoch-Schönlein Purpura, Pseudothrombocytopenia, or combinations thereof.

In certain embodiments the drug is used to control progression of an inflammatory disease. In certain embodiments, the drug is used to control progression of one or more of the following: multiple sclerosis, Crohn's disease, asthma, psoriasis and rheumatoid arthritis. In certain embodiments, the drug is used to control progression of disease caused by one or more of the following: organ transplant, stroke, myocardial infarction and traumatic shock.

Blood Sample

In some embodiments, the leukocyte adhesive function assay requiring only a small amount of whole blood, such as 5, 10, 15, 20, 30, 40, 50, 60, 70, 80, 90, 100, 120, 150, 200, 300, 400, 500, 750 and 1.000 μl.

The method may comprise subjecting more than one blood sample obtained from the subject to a leukocyte adhesive function assay or more than one leukocyte adhesive function assay.

The method may include the step of isolating the blood sample from the subject. This may be achieved in various suitable ways. For example, blood may be obtained by pricking a finger and collecting the drop/s, or by venepuncture. In certain embodiments a drop of blood may be used for the method. In certain embodiments, less than about 100 μL of blood may be required for the leukocyte adhesive function assay, such as 5, 10, 15, 20, 30, 40, 50, 60, 70, 80, 90, or 100. In certain embodiments, less than about 100 μL of blood may be required for the leukocyte adhesive function assay, such as less than 5, 10, 15, 20, 30, 40, 50, 60, 70, 80, 90, or 100.

In some embodiments, the blood sample may be whole blood, whether processed or not. In some other embodiments the blood sample is a processed sample whereby one or more components of whole blood have been separated from each other. That is, in some embodiments the blood sample may be whole blood, and in other embodiments the blood sample may comprise or one or more white blood cell components of (processed/treated) whole blood.

In certain embodiments, blood components are not separated from the whole blood sample so as to mimic blood in vivo. In certain embodiments isolated blood cells, cultured blood cells and/or blood cell lines may be used.

Anticoagulants that may be used to collect and store blood samples may include, but are not limited to, heparin, EDTA, ACD, citrate, Hirudin, sodium polyanethol sulfonate and potassium oxalate/sodium fluoride.

Leukocyte Adhesive Function Assay and Device

The leukocyte adhesive function assay may be various suitable type of assay. The method may comprise carrying out more than one leukocyte adhesive function assay, to obtain one or more results. The leukocyte adhesive function assay may include one or more specific tests to provide a collective result.

In certain exemplary embodiments, the leukocyte adhesive function assay results may be semi-quantitative and/or quantitative.

The leukocyte adhesive function assay may achieve one or more of the following: characterising leukocyte cell recruitment; characterising leukocyte cell tracking; characterising leukocyte cell migratory behaviour—in a quantitative manner.

In some embodiments, the leukocyte adhesive function assay may entail quantitatively determining leukocyte migration. This may include detecting, measuring or observing leukocyte cell tethering, rolling, slow rolling, firm adhesion, crawling and/or trans-endothelial migration. In some embodiments, the leukocyte adhesive function assay may entail detecting, measuring or observing leukocyte cell average speed, displacement, acceleration, deceleration, directionality, dwell time and/or straightness.

Interacting leukocytes may be characterised by way of velocity distribution. For example, interacting leukocytes may be divided into five interaction types according to cell mean speed (Sₘₑₐₙ): static cells (Sₘₑₐₙ<5 μm/min), crawling cells (Sₘₑₐₙ=5-20 μm/min), slow rolling cells (Sₘₑₐₙ=20-300 μm/min), and rolling cells (Sₘₑₐₙ=300-6000 μm/min). In addition, a histogram may be used to show the distribution of cell velocity.

In certain embodiments, the leukocyte adhesive function assay entails detecting, measuring and/or observing leukocyte migration under realistic physiological conditions.

In some embodiments the assay allows for simultaneous detection of different leukocyte subsets.

In certain embodiments, the leukocyte adhesive function assay involves a flow assay.

As part of the leukocyte adhesive function assay, the blood sample may be premixed, pre-treated or pre-incubated with one or more cell stains, one or more chemicals (e.g. such as manganese which induces α4 integrin activation), one or more of the drugs (with or without a detectable moiety), one or more antibodies, and/or one or more detectable moieties or other reagents or agents.

In some embodiments, the method may comprise treating subject (human or animal) blood with one or more drugs, reagents or agents in vitro, then carrying out the leukocyte adhesive function assay.

In some embodiments, the method may comprise administering a subject with one or more drugs, reagents or agents in vivo, then carrying out the leukocyte adhesive function assay.

In some embodiments, the leukocyte adhesive function assay may assess leukocyte migration under realistic physiological conditions.

In some embodiment, the leukocyte adhesive function assay may utilise leukocytes labelled with an antibody conjugated to a fluorophore or other detectable moiety. In some embodiments, the assay may entail detecting different subsets of leukocytes with subset-specific antibodies conjugated to different fluorophores. For example, an antibody or antibody cocktail and/or stain may be added to the blood sample. For example, fluorescently labelled antibodies against specific leukocyte membrane markers may be added to the blood sample before performing a flow assay.

The leukocyte adhesive function assay or flow assay may utilise a suitable type of equipment for detecting, measuring or observing leukocyte migration etc, including for detecting, measuring or observing leukocyte migration etc under realistic physiological conditions. Examples of suitable microfluidic assays and/or devices are described in the following documents: U.S. Pat. Nos. 8,940,494; 8,380,443; 7,326,563; WO 92/21746; Vaidyanathan R I, Shiddiky M J, Rauf S. Dray E. Tay Z, Trau M.; Tunable “nano-shearing”: a physical mechanism to displace nonspecific cell adhesion during rare cell detection; Anal Chem. 2014 Feb. 18: 86(4):2042-9. doi: 10.1021/ac4032516. Epub 2014 Feb. 4—the entire contents of which are incorporated herein by way of cross-reference.

A microfluidic device may be used for carrying out a flow assay. In some embodiments the flow assay entails using a microfluidic device having one, two, three, four, five, six or more microfluidic channels, for example, for detecting different leukocyte subsets and/or adhesion molecules.

In some embodiments the blood sample may be assayed in a microfluidic device to mimic blood flow in vivo.

In some embodiments the flow assay entails pulling or pushing the blood sample into one or more microfluidic channels, for example using a syringe pump, preferably at a shear stress of approximately 0.5 to 30 dyne/cm², including 0.2, 0.5, 1, 2, 3, 4, 5, 6, 7, 8, 9, 10, 15, 20, 25, 30, 40, 50, 60, 70, 80, 100, 150, 200, 300 dyne/cm².

The leukocyte adhesive function assay may allow for visual analysis for characterising leukocyte cell migratory behaviour, characterising leukocyte cell tracking, or characterising leukocyte cell recruitment by the endothelial adhesion molecule. Visual analysis may be carried out in any suitable way. For example, visualisation may be achieved using a microscope and image recorder (e.g. video or time-lapsed photography). Leukocyte migratory behaviour, tracking, recruitment etc may be analysed by way of computer analysis of the images captured by the image recorder. The kinds and numbers of adhesive and/or non-adhesive leukocytes may be determined and their individual velocities/behaviours may be recorded and analysed in a quantitative manner.

In some embodiments the leukocyte adhesive function assay entails acquiring images at high frame rate over a period of time sufficient to capture all types of leukocyte cell interactions. For example, the assay may entail acquiring images at 2 frames per second for 5 minutes to capture types of cell interactions. In some embodiments the leukocyte adhesive function assay may entail capturing detailed 3D movement of leukocytes. In some embodiments the leukocyte adhesive function assay entails recording a fluorescence microscopy time series.

The leukocyte cell kinetic parameters are derived in the following manner: The recorded image time series provides x, y and z (position) and t (time) coordinates of each detected, interacting leukocyte cell. By linking localizations of the same leukocyte cell between several frames using mathematical algorithms such as ‘nearest neighbour’, cells may be tracked over time and various parameters obtained to characterize cell motion (such as track direction, length, displacement, duration, straightness, mean speed, acceleration/deceleration, directed/confined/random motion type). Those parameters may then be used to differentiate motility behaviour of different leukocyte cell subpopulations or changes in motility upon drug treatment.

Alternatively, other methods for detecting leukocyte cells may be used, as described for example in: Nan Sun, Yong Liu, Ling Qin, Guangyu Xu, and Donhee Ham. September 2012, Solid-State and Biological Systems Interface Solid-State Device Research Conference (ESSDERC); DOI: 10.1109/ESSDERC.2012.6343324; http://people.seas.harvard.edu/˜donhee/ESSDERC-ESSCIRC-2012-Ham.pdf (the entire contents of which are hereby incorporated by way of cross-reference).

The endothelial molecule may be in the form of, for example, a recombinant protein bound to a support or substrate. Put another way, in some embodiments the assay may involve using a plurality of endothelial molecules fixed to a support or substrate (perhaps including a lipid bilayer), and in other embodiments the assay may involve using actual cells expressing such endothelial molecules. With regard to endothelial molecules immobolised to a support or substrate, a number of techniques are referenced in Dohyun Kim and Amy Herr, Protein immobilization techniques for microfluidic assays, Biomicrofluidics, 2013, and is hereby incorporated by reference in its entirety. Also, such molecules are described in the following documents, the entire contents of which are incorporated herein by way of reference: U.S. Pat. Nos. 8,940,494; 8,380,443; 7,326,563; and WO 92/21746.

Endothelial molecules that may be used as adhesive substrate (i.e., bound to a support or substrate) in the leukocyte adhesive function assay include but not limited to one or more of the following:
1. Adhesion molecules as already described herein;
2. Chemokines as mentioned herein;
3. Purified antigens and artificial antigen-presenting cell system:
   a. Purified antigens: i) alpha, beta and epsilon toxins and ii) antigen CFA/I
   b. Artificial antigen-presenting cell systems disclosed in 1) Anna Thomas and Marcela Maus, e α1, “A Cell-Based Artificial Antigen-Presenting Cell Coated with Anti-CD3 and CD28 Antibodies Enables Rapid Expansion and Long-Term Growth of CD4 T Lymphocytes” Clinical Immunology, 2002, and 2) Turtle, C., Riddell, S., “Artificial antigen presenting cells for use in adoptive immunotherapy” Cancer J, 2010 and these references are hereby incorporated by reference in its entirety;
4. Other molecules (including proteins) that may regulate cell-cell interactions; and
5. Chemokine receptors as disclosed herein.

In some embodiments, the leukocyte adhesive function assay may entail detecting, measuring or observing the interaction between leukocyte-expressed PSGL-1 (P-selectin glycoprotein ligand-1) and its endothelial molecule, P-selectin and/or E-selectin.

In some embodiments, the leukocyte adhesive function assay may entail quantitative assessment of α4 integrin adhesion functions.

In some embodiments, the leukocyte adhesive function assay may entail detecting, measuring or observing increased leukocyte α4 integrin expression and activity.

In some embodiments, the leukocyte adhesive function assay may entail measuring, detecting and/or observing the interaction between leukocyte α4 integrin and endothelial VCAM-1.

In some embodiments, the leukocyte adhesive function assay may entail detecting, measuring and/or observing the interaction between CD11a (αL integrin) and ICAM-1.

In some embodiments, the leukocyte adhesive function assay may entail detecting, measuring or observing the interaction between CD11b (αM integrin) and ICAM-1.

In some embodiments, the leukocyte adhesive function assay may entail detecting, measuring and/or observing the interaction between α4β7 integrin and MAdCAM-1.

In some embodiment, the leukocyte adhesive function assay may entail detecting, measuring and/or observing the interaction between intercellular adhesion molecule-1 (ICAM-1) and/or vascular cell adhesion molecule-1 (VCAM-1) and their leukocyte adhesion molecule.

In some embodiment, the leukocyte adhesive function assay may entail detecting, measuring and/or observing the interaction between leukocyte (32 integrin and its endothelial molecule.

The leukocyte adhesive function assay may entail measuring one or more specific subsets of leukocytes, such as CD4, CD8 and CD15 cells.

In some embodiments, the leukocyte adhesive function assay may entail detecting, measuring or observing leukocyte migratory behaviours on cytokine or chemokine (e.g. TNFα and IL-4) activated primary endothelial cells (e.g. HUVEC) or immobilised endothelial cell lines (e.g. human microcirculation endothelial cells (HMEC)).

In some embodiments, the leukocyte adhesive function assay may entail detecting, measuring and/or observing the effects of the drug Natalizumab on leukocyte interaction with TNFα activated HUVEC.

In some embodiments, the leukocyte adhesive function assay may entail detecting, measuring and/or observing Natalizumab-specific binding to α4 integrin on leukocytes.

In some embodiments, the leukocyte adhesive function assay may entail measuring, detecting and/or observing the inhibitory) effects of Natalizumab on α4 integrin functions.

In some embodiments, the leukocyte adhesive function assay may entail simultaneously detecting, measuring and/or observing different leukocyte subsets by labelling the subsets with specific membrane markers. Such markers may be antibodies conjugated to different fluorophores.

The leukocyte adhesive function assay may include one or more controls. The nature of the control/s employed may depend on the nature of the assay and the nature of the method employing the essay. For example, the control may be a blood sample obtained from a healthy individual who does not have a disease or disorder (e.g. an inflammatory or autoimmune disease). For example, the control may be a blood sample obtained from an individual who is not under medical treatment with drugs (e.g. anti-inflammatory drug). For example, the control may be a blood sample obtained from the subject prior to being administered the drug, prior to receiving drug treatment, or prior to being subjected to a dosage regimen or during a dosage regimen. The control may be a blood sample comprising pooled blood samples from different individuals (cohort).

In some embodiments the method/leukocyte adhesive function assay may entail carrying out the following steps: 1. Pre-coating a flow channel with an endothelial molecule; or if in endothelial cell models, seed and culture cells in the flow channel, and activate the expression of endothelial adhesion molecules by treating the cells with a reagent or inflammatory cytokines or chemokines, e.g. TNFα; 2. Incubating the flow channel without or with a drug at various doses (e.g. small molecule, antibody etc), which alters endothelial adhesion molecule functions; 3. Collecting blood from a subject; and, 4. Performing leukocyte adhesive function assays at various time points post-drug treatment to determine the drug effects (by comparison to drug-free controls). An example of a suitable assay is described in.

Dosage Regimen

The dosage regimen may typically depend on the nature of the drug, the disease condition and/or the subject's characteristics. Optimising the drug dosage regimen may involve, for example, modifying the: route of drug administration; galenic drug formulation; drug unit dose; frequency of administration/length of time between administrations; drug loading dose; and/or length of treatment—as required judging by the assay result.

Optimising the drug dosage regimen (e.g. of an immune-suppressive drug) may involve allowing the drug serum level to decrease below maximal efficacy each, a substantial portion or a portion of the dosing cycles, but without comprising, or substantially compromising, the drug efficacy. Once below maximal efficacy threshold, the immune response of the subject may be restored for certain a period of time before drug re-dosing, as detailed in Example 7.

In certain exemplary embodiments, optimising the drug dosage regimen and/or determining a minimum effective drug dose may mean that only a minimum amount of the drug need be administered and/or the time between sequential drug administrations may be lengthened or shortened as required. In certain exemplary embodiments, optimising the drug dosage regimen and/or determining a minimum effective drug dose may mean that pathological or fatal side-effect due to the drug may be minimised and/or eliminated. For example, the risk of progressive multifocal leukoencephalopathy (PML), a fatal side effect of Natalizumab therapy, may be reduced.

Subject

The subject may be a mammal or any other suitable type of animal. Mammals include humans, primates, livestock and farm animals (e.g. horses, sheep and pigs), companion animals (e.g. dogs and cats), and laboratory test animals (e.g. rats, mice and rabbits). In certain embodiments, the subject may be human.

Treating a Subject

The subject may be treated in a conventional way known for that particular disease. In addition, the subject may be treated in non-conventional way. For example, based on the results from LAFA analysis, the suitability of a drug for the treatments of a subject with certain diseases may be projected, even though the drug is usually not used for this particular disease.

Natalizumab

Certain exemplary embodiments, find application with regard to Natalizumab treatment of subjects with multiple sclerosis. For example, Natalizumab re-dosing in subjects having multiple sclerosis is usually carried out once every four weeks after the initial infusion. The standard dose of Natalizumab is 300 mg per subject per infusion. Optimising the drug dosage regimen may involve, for example, reducing the amount of drug administered and/or increasing the length between administrations—as required judging by the assay result.

In some embodiments, the method may be carried out as a blood test, performed at various time points post-Natalizumab infusion. Accordingly, the assay results may be used to determine the need of Natalizumab re-dosing. The blood test may be conducted in individual subjects to ensure drug effectiveness, facilitating the development of optimal/personalised treatment regimen for individual subjects. The blood test may be conducted for many subjects for patient stratification.

Recent clinical data from extended dosing interval (EDI) studies showed extended interval of Natazumab therapy (up to 8 weeks) may not only suppress disease progression as efficiently as standard intervals (4 weeks), but also significantly reduce the risk of PML. These finding suggest that EDI reduces the risk of PML although the mechanisms remains unknown. It is reasonable to propose that once the serum level of Natalizumab reaches below a certain threshold (e.g., the 100% saturation threshold), a minimal immune response may be restored to reduce the risk of JCV infection. The methods described herein may quantitatively assess Natalizumab effectiveness, precisely indicating the drug saturation level and the need for re-dosing. Thus, the method may facilitate the restoration of immune response without comprising the drug efficacy, allowing enough immune response each, a substantial portion or a portion of the dosing cycles to effectively eliminate, mitigate and/or reduce risk of PML.

Certain exemplary embodiments relate to the use of a leukocyte adhesive function assay for detecting activation of a drug target, which may then be used to predict the ability of the drug to control disease progression. The method/assay may also be used to predict whether the drug may be used to control the progression of yet other diseases not known to be treatable using that drug. For example, Natalizumab may for some subjects be a useful drug other than for treating multiple sclerosis and Crohn's disease if an α4 integrin activation is detected in a subject.

Despite early success in controlling disease activity in multiple sclerosis (MS) patients, it was noted that 25.8% and 17.1% of Natalizumab recipients poorly and only partially responded to the treatments. Up to date, the mechanism underlining such low drug effectiveness rate remains unknown. Although the pathological role of α4 integrin was established in MS, it is highly unlikely that the levels of α4 integrin activation would be the same in highly heterogeneous patient population. This notion is supported by the findings that the magnitude of the therapeutic efficacy of Natalizumab varied dramatically between individual MS patients, suggesting that the pathological role of α4 integrin may differ within these patients. Accordingly, patients with high level of α4 activation would be more likely to respond to Natalizumab therapy, comparing to patients with low or no α4 integrin activation. Certain exemplary embodiments may be capable of quantitatively assessing the activation α4 integrin, providing an excellent tool to predict how likely a subject would respond to Natalizumab therapy, facilitating patient stratification.

In certain exemplary embodiments, there is provided a method of generating a leukocyte adhesion profile for a subject, said method comprising the steps of:
subjecting at least one blood sample obtained from the subject to at least one leukocyte adhesive function assay in vitro so as to quantitatively assess the adhesion functions of different leukocyte subsets to one or more different endothelial molecules at substantially the same time; and
using the assay result for: identifying leukocyte abnormalities: determination of personalised pathogenesis; identification of new disease markers for diseases; identifying early signs of disease; disease prediction; disease prevention; assisting with early an accurate diagnosis; developing an effective and personalised treatment for the subject; monitoring the health (healthy status) of the subject; grouping subjects regardless of disease; or developing a treatment for the subject regardless of disease diagnosis.

The method (or methods) may be carried out on a blood sample obtained from a single subject. The method (or methods) may be carried out on blood samples obtained from a plurality of different subjects. The subject (or subjects) may be a healthy subject. The subject (or subjects) may have a disease.

The assay (or assays) may be carried out on a blood sample of a subject, and the blood sample may be a whole blood sample or processed blood sample.

The leukocyte adhesive function assay may be a flow (cell) assay for quantitating leukocyte cell migratory behaviour, leukocyte cell tracking, or leukocyte cell recruitment by the one or more endothelial adhesion molecules and/or other related molecules and/or cells that are expressing these molecules.

The one or more endothelial molecules is/are in the form of a recombinant protein bound to a support or substrate, or a cell system overexpressing the one or more endothelial adhesion molecules.

Suitable types of leukocytes include, for example, those described disclosed herein.

Suitable types of leukocyte adhesion molecules or other binding molecules of the leukocyte include, for example, those disclosed herein.

The one or more endothelial molecules may include, for example, those disclosed herein.

The method (or methods) may involve assaying the adhesion of different leukocyte subsets in individual flow channels, which may be pre-coated with specific endothelial molecule substrates. As a result, cell migration profiles for each, a substantial portion or a portion of adhesion molecule on a specific leukocyte subset may be generated.

The assay of the method (or methods) may comprise the step of identifying a drug target and then choosing an appropriate drug for controlling progression of the disease based the drug target.

The assay (or assays) of the method (or methods) may comprise the step of identifying a drug target and then choosing an appropriate drug for controlling progression of the disease based on a reference database of drug targets and drugs for those targets. In this way, the disease itself need not actually be diagnosed and/or identified and/or known.

The assay (or assays) of the method (or methods) may comprise the step of building a database of drug targets and drugs.

The assay (or assays) of the method (or methods) may comprise the step of assaying for more than one drug target at the one time (e.g., 2, 3, 4, 5, 6, 7, 8, 9 or 10 drug targets or more).

The method (or methods) may be a high throughput assay, testing for a plurality of drug targets at the one time.

The method (or methods) may have other features as described for other embodiments.

Any of the features described herein may be combined in any combination with any one or more of the other features described herein within the scope of the invention.

Materials and Methods for Certain Exemplary Embodiments

To realistically recapitulate human microcirculation, unprocessed human whole blood was used in a microfluidic system to mimic blood flow in vitro. Different leukocyte subsets were simultaneously detected by labelling the cells with specific membrane markers, using antibodies conjugated to different fluorophores. The interaction between leukocyte α4 integrin and endothelial VCAM-1 was examined, as was leukocyte recruitment on VCAM-1 substrate, and cell kinetics was employed to characterise cell migratory behaviours, allowing a quantitative assessment of α4 integrin adhesion functions.

Materials and Methods

Antibodies, Chemical and Reagents

Human recombinant VCAM-1 and TNFα were purchased from R&D Systems (Minneapolis, Minn.). Antibodies (Abs) against human leukocyte surface molecules, CD4-Alexa488, CD8-PE, CD15-APC and CD16-BV510, were obtained from BD Biosciences (San Diego, Calif.). Natalizumab (Tysabri) was purchased from Biogen (Cambridge, Mass.). The alternative anti human α4 integrin Ab (Clone: 7.2R) was purchased from R&D Systems.

Flow Channel System to Study VCAM-1 Dependent Leukocyte Recruitment

Human blood was collected from healthy volunteers in lithium heparin blood collection tubes, stored at room temperature, and used within 4 hours after blood collection. To study VCAM-1 dependent leukocyte recruitment, a Polymethyl methacrylate (PMMA)-bottom microfluidic chip (channel Width×Depth×Length: 1,000×200×18,000 μm) purchased from Microfluidic ChipShop (Jena, Germany) was employed. Recombinant human VCAM-1 protein (10 μg/ml) was gently loaded into the channels and incubated overnight at 4° C. To identify specific leukocyte subsets, 100 μl whole blood was mixed with the following antibody (Ab) cocktail for 5 minutes (min) at room temperature anti-CD4-Alex488 (1:50 dilution), anti-CD8-PE (1:66.7) and anti-CD15-APC (1:33.3). The blood was then pulled into the microfluidic channels by a syringe pump (Harvard Apparatus, Holliston, Mass.) at a shear stress of 1.5 dyne/cm². To activate α4 integrin, whole blood was treated with 5 mM Manganese chloride (Sigma, St Louis, Mo.) for 5 min at room temperature. To study the inhibitory effects of Natalizumab on α4 integrin functions, blood was incubated with various doses of Natalizumab for 5 min at room temperature before being used for the flow assay. In those experiments where there was a need to distinguish neutrophils (CD15⁺CD16⁺) from eosinophils (CD15⁺CD16⁻), an anti-human CD16-BV510 Ab (1:50) was also included.

Fluorescence microscopy time series were recorded on a DeltaVision Widefield microscope (Applied Precision, Issaquah, Wash.) with a 10× objective and Olympus IX71 base under critical illumination, with 20 MHz camera readout speed and 4×4 pixel binning to facilitate high speed image acquisition. Data acquisition was recorded at 2 frames per second for 10 minutes, at the centre of the channel (9,000 μm from the channel inlet). The experiments were performed in a temperature controlled and equilibrated environment (37 degrees Celsius and 5% CO₂).

Cell Tracking and Data Analysis

Cell tracking was accomplished using Imaris (Bitplane AG) software. Cells were tracked automatically by detecting quality-filtered fluorescent spots in each frame and then linked with a maximum distance of 30 μm and maximum gap size of 2. The tracks were subsequently checked manually and corrected for errors. Track parameters and statistics such as dwell time, straightness and mean speed were then exported for statistical analysis in GraphPad Prism.

The interacting cells were divided into five interaction types according to cell mean speed (Sₘₑₐₙ): static cells (Sₘₑₐₙ<5 μm/min), crawling cells (Sₘₑₐₙ=5-20 μm/min), slow rolling cells (Sₘₑₐₙ=20-300 μm/min), and rolling cells (Sₘₑₐₙ=300-6000 μm/min). The half maximal inhibitory concentration (IC50) values of Natalizumab were calculated using R Studio. Common origin graphs were obtained by with R Studio by normalizing the detected tracks to the same coordinates of origin (0.0 μm).

Flow Chamber Assay to Study Leukocyte Recruitment by Endothelial Cells

To study leukocyte recruitment by human blood vessel endothelial cells, a parallel plate flow chamber system (Glycotech, Gaithersburg, Md.) were used. Briefly, heparinised human blood was first diluted 1:10 with Hank's balanced salt solution (HBSS) containing Ca²⁺ and Mg²⁺. To detect the types of cell interactions (including fast rolling), high frame rate recording were used. Whole blood was labelled with Hoechst33342 only for 5 min at room temperature before being used for the flow assay. Images were acquired only in the Hoechst channel at a high frame rate (2 frames per second) for 5 min. For Natalizumab experiments, blood was treated with either 10 or 300 ng/ml of Natalizumab at room temperature for 5 min. Primary human umbilical vein endothelial cells (HUVEC) were cultured to 100% confluence before being trypsinised and seeded on a fibronectin-coated glass coverslip. The cells were then cultured for an additional 24 hours before being activated with 10 ng/ml of human tumor necrosis factor α (TNFα) overnight. After this, the flow chamber was carefully placed onto the coverslip and the blood was then pulled into the chamber at a shear rate of 150 s⁻¹ by the syringe pump. Cell tracking and data analysis were performed as mentioned herein.

To further study cell migration behaviours in different leukocyte subsets, Hoechst33342 plus an antibody cocktail, containing anti-CD4-Alex488, anti-CD8-PE and anti-CD15-APC, were added to the whole blood, which were then incubated for 5 min at room temperature before the flow assay. The interaction between leukocytes and HUVEC were recorded using a 20×0.75 NA objective. Four-channel 3D z-stacks were recorded over a range of 20 μm at 1 μm intervals to account for the 3-dimensionality of the endothelial cell coated surface. A low frame rate (1 frame per 30 secs) was used to detect the 3D movement of slow moving cells, including static, crawling and slow rolling cells. Images were recorded at three different positions arranged perpendicular to the flow direction. For visualization and data pre-processing, 3D data sets were deconvolved using SoftWoRx software (Applied Precision).

### Example 1: Mn²⁺ Activates α4β1 Integrin Adhesive Functions

In this exemplary embodiment, to assess the ability of leukocyte to interact with endothelial VCAM-1, a microfluidic system was employed to mimic blood microcirculation in vitro, which consists of a microfluidic pump and microfluidic chips. The bottom of the chip was pre-coated with VCAM-1 protein (10 μg/ml) at 4° C. overnight, and whole blood was then perfused through the channel at a flow rate of 10 μl/min, driven by the microfluidic pump. Unless otherwise stated the protocol used is set for in Appendix I.

Simultaneous Detection of VCAM-1 Dependent Recruitment of CD4, CD8 and CD15 Cells

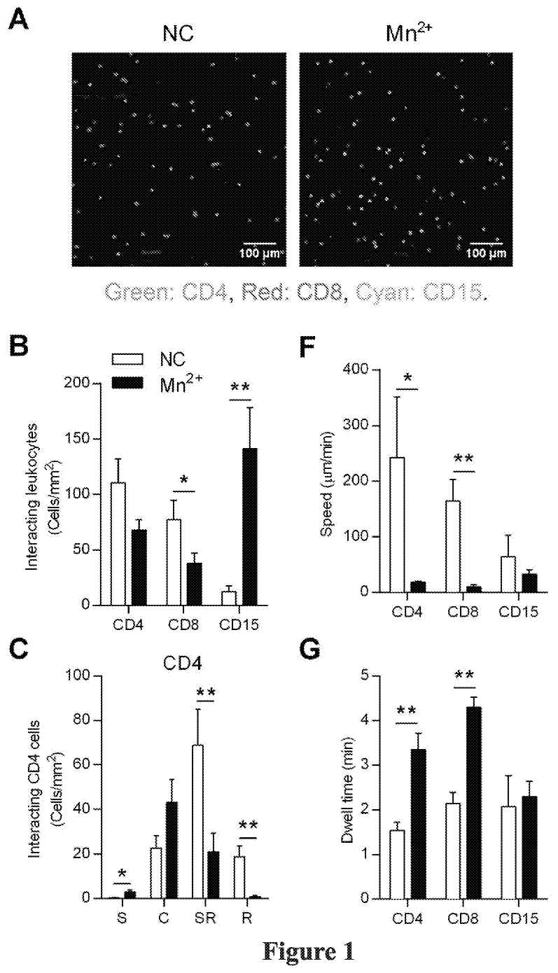

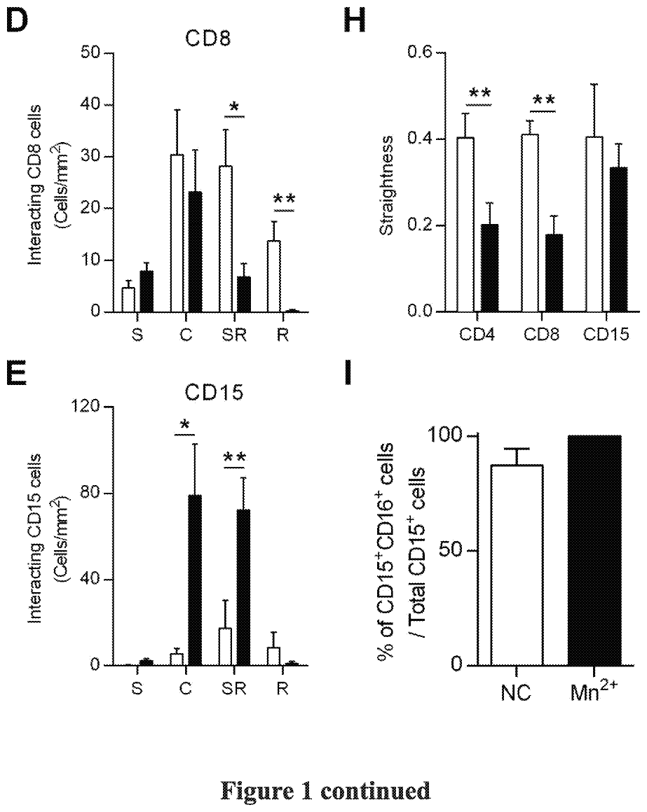

In the present example, to avoid cell isolation and achieve simultaneous detection of multiple specific leukocyte subsets, fluorescently labelled antibodies against specific leukocyte membrane markers were added to human whole blood before performing the flow assay. As shown in FIG. 1A, CD⁴⁺ (Green), CD8⁺ (Red) and CD15⁺ (Cyan) subpopulations of leukocytes may be clearly distinguished. The numbers of interacting CD4 and CD8 cells are similar, whereas the number of CD15 cells is significantly lower (p<0.05) than CD4 and CD8 cells (FIG. 1B). In fact, no interacting CD15 cells were detected in 50% of the blood samples analysed. These results shoes that that VCAM-1 preferentially recruits CD4 and CD8 cells compared to CD15 cells in healthy control blood.

Mn²⁺, a van integrin activator, induces α4β1 integrin activation, therefore leading to an increased binding activity to its ligand, VCAM-1. To test the ability of the system to detect Mn²⁺-induced leukocyte adhesive functions, whole blood was treated with Mn²⁺ before being used for the flow assay. Compared to untreated blood, Mn²⁺ has no significant effect on the number of interacting CD4 cells, while a >50% reduction in CD8 cells was detected (FIG. 1B). In contrast, Mn² treatments induced an almost 5 fold increase in the number of interacting CD15 cells (FIG. 1B), showing a functional role of α4β1 integrin on CD15 cells to support cell interaction with VCAM-1.

To characterise the cell migratory behaviours, a range of cell kinetic parameters were utilised to assess the ligand binding activity of α4β1 integrin. According to their mean speed of migration, total interacting cells were divided into static (<5 μm/min), crawling (5-20 μm/min), slow rolling (20-300 μm/min) and rolling cells (300-6000 μm/min). In non-activated blood, very little static CD4 cells (0.2±0.2 cells/mm) were detected, while the majority of interacting CD4 cells are slow rolling cells (68.8±16.1 cells/mm², FIG. 1C). Compared to untreated controls, Mn²⁺ treatment significantly increased the number of static CD4 cells to 2.9±1.0 cells/mm², p<0.05. In contrast, the number of slow rolling and rolling CD4 cells was markedly decreased by Mn²⁺ (FIG. 1C). These findings show that Mn²⁺ enhances α4β1 integrin ligand binding ability and cell-VCAM-1 interaction, leading to a reduction of cell migration speed. In CD8 cells, Mn²⁺ significantly decreased the number of slow rolling (p<0.05) and rolling (p<0.01) CD8 cells, whereas no effect on static and crawling cells was observed (FIG. 1D). These findings show that Mn² treatments inhibit the ability of α4β1 integrin to support CD8 cell rolling. In addition, the number of crawling and slow rolling CD15 cells was dramatically increased by Mn²⁺ (FIG. 1E), showing a role of α4β1 integrin in regulation of CD15 cell adhesive functions.

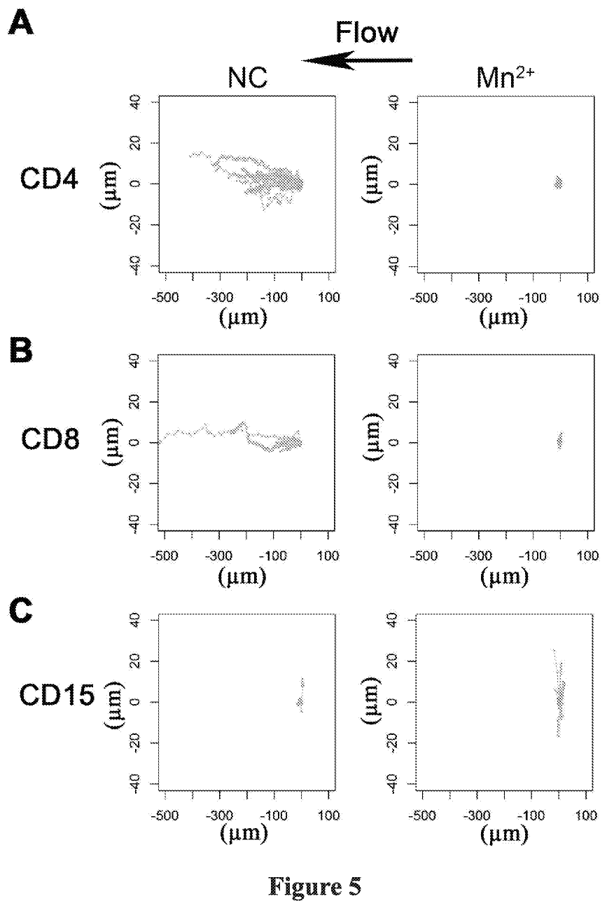

To determine the overall effects of Mn²⁺ on the speed of interacting cells, the average speed of three leukocyte subsets in the presence and absence of Mn²⁺ were compared. Mn²⁺ treatments significantly reduced the speed of CD4 and CD8 cells (FIG. 1F), compared to no Mn²⁺ controls. Consistently, the common original graphs show that the motility of CD4 and CD8 cells was markedly inhibited by Mn²⁺ (FIGS. 5A and 5B). These results are in line with the findings in FIGS. 1C-D, showing suppressive effects of Mn²⁺ on cell migration.

Additionally, Mn²⁺ treatments significantly increased the dwell time of interacting CD4 and CD8 cells (FIG. 1G), whereas the straightness of CD4 and CD8 cells were significantly decreased by Mn²⁺ (FIG. 1H). Together, these results show that Mn²⁺ treatments induce α4β1 integrin adhesive function, allowing a stronger cell interaction with VCAM-1. On the other hand, speed, motility, dwell time and straightness of interacting CD15 cells were not affected by Mn²⁺ (FIGS. 1F-1H, FIG. 5C). These findings show that Mn²⁺ treatments enhanced the recruitment of CD15 cells without affecting their migratory behaviours.

### Example 2: The Use of Multiple Membrane Markers to Identify Specific Leukocyte Subsets

The present example is directed to the identification of CD15 and CD16 double positive neutrophils in the assays, according to certain exemplary embodiments. The protocol set forth in appendix I was followed with following modifications made to perform the experiments:

1. 3 μl of anti-human CD15-APC (BD, Cat #: 551376) and 2 μl of anti-human CD16-BV510 (BD, Cat #: 563830) were added to 100 μl of human whole blood, and incubated at room temperature for approximately 5 minutes, before being used for the assay,
2. A macro called “Multi_Channel.ijm” was used to track CD15CD16 double positive cells in Fiji software.

Most of the interacting CD15 cells are neutrophils. Eosinophils are a small population of granulocytes that are known to not only have high expression level of α4 integrin, but also be positive for CD15 expression. To determine if the interacting CD15 cells are neutrophils or eosinophils, an additional fluorescently-labelled antibody against human CD16 was introduced to the assay, allowing the distinction between neutrophils (CD15CD16⁺) and eosinophils (CD15⁺CD16). As shown in FIG. 1I, almost all interacting CD15 cells are also CD16 positive, indicating that most of these CD15 cells are neutrophils.

This strategy may be used to detect other specific leukocyte subsets, using 2, 3, 4 or more membrane markers. For example, CD14 and CD16 double positivity may be used to identify inflammatory monocytes, whereas CD4 and CD25 double positivity may be used to identify CD4 T regulatory (Treg) lymphocytes. Different fluorophore-conjugated antibodies against CD14 and CD16 (or CD4 and CD25 for Treg) may be added to the whole blood before being used for LAFA analysis. As a result, CD14CD16 double positive cells (inflammatory monocytes) may be detected by the microscope and their adhesive function may then be quantitatively assessed as described in Example 1.

### Example 3: Semi-Quantitative Assessment of Basal Inflammatory Status of α4β1 Integrin

The present example is directed to showing semi-quantitative assessment tools that may be used in certain exemplary embodiments. In disease subjects, compared to healthy subjects, the portion of activated α4β1 integrin molecules is increased, resulting in enhanced the ability of white blood cell to bind to α4β1 integrin endothelial ligand (e.g. VCAM-1), and increased leukocyte recruitment and/or inflammatory response.

The following criteria are used to define a healthy subject or healthy subjects:
1. Overtly healthy, as determined by medical evaluation including medical history.
2. Women who are NOT pregnant or currently lactating
3. Not being diagnosed of any autoimmune, inflammatory, hematologic and vascular disorders
4. Currently NOT taking prescribed medication, except for contraceptives
5. Currently NOT taking over-the-counter medications that may affect blood cell functions, including anti-histamine drugs, aspirin etc. Vitamin supplements are acceptable for this study
6. Currently do not have active cold, fever or known allergic reactions
7. No recent (last 5 years) smoking history.

As shown in FIGS. 1G to 1H, a range parameter may be used to characterise the basal inflammatory status of α4β1 integrin. The results herein shown that Mn²⁺ treatments significantly decreased the straightness and speed of CD4 and CD8 cells on VCAM-1 substrate. It is known that α4β1 integrin function is activated in subjects with autoimmune disease, but it is unclear how active the α4β1 integrin is in individual subjects. As shown in FIG. 1H, straightness is the best and most robust parameters that were affected by Mn²⁺ treatments, suggesting that the reduction of straightness may be a good marker for Mn²⁺ induced α4 integrin activation. As Mn²⁺ treatments chemically activated α4β1 integrin to a maximal level, it is highly likely that the straightness value of leukocytes from a subject or subjects will fall between the values of controls and Mn²⁺ treated samples. The average straightness value of untreated CD4 cells is 0.40±0.06, which was reduced to 0.16±0.03 by Mn²⁺ treatments. Thus, if the average straightness values of control and Mn²⁺ treated leukocytes are arbitrarily set to be 10 and 1 (Relative Straightness Index (RSTI)) respectively, it is highly likely that the RSI values of leukocytes from a subject or subjects will fall between 10 and 1, offering a semi-quantitative approach to assess the basal inflammatory status and/or adhesive functions of α4β1 integrin. In this case, the closer the RSTI is to 1, the more active is the patient's α4β1 integrin, the higher is the basal inflammatory status. Thus, the basal inflammatory status of α4β1 integrin in individual patients may be determined.

In addition, in FIG. 1F, if the average white blood cell speed of control and Mn²⁺ treated leukocytes are arbitrarily set to be 10 and 1 (also referred to as Relative Speed Index (RSI)) respectively, the RSI value offers a semi-quantitative tool to assess α4β1 integrin adhesive functions in an individual subject. For example, in CD4 cells, the average white blood cell speed is 135.7 and 17.9 μm/min in the absence and the presence of Mn²⁺, these two data points may be used to define 10 and 1 respectively for the RSI values for CD4 cells. If the CD4 cell RSI value of a subject falls between 10 and 1, the closer the RSI is to 1, the more active is the α4β1 integrin, and the higher is the basal cell inflammatory status.

Similarly, in FIG. 1G, if the average dwell time of control and Mn²⁺ treated leukocytes are arbitrarily set to be 1 and 10 (also referred to as Relative Dwell Time Index (RDTI)) respectively, the RDTI value will also offer a semi-quantitative tool to assess α4β1 integrin adhesive functions. For example, in CD4 cells, the average white blood cell dwell time is 1.53 and 3.52 minutes in the absence and the presence of Mn²¹, these two data points may be used to define 10 and 1 respectively for the RDTI values of CD4 cells. In this case, the closer the RDTI is to 1, the more active is α4β1 integrin.

The basal inflammatory level of α4β1 integrin may be used as a semi-quantitative tool to assess basal level of α4β1 integrin activation and/or basal inflammation status of a subject or subjects, such as patients with multiple sclerosis, Crohn's disease, colitis, atherosclerosis, autoimmune thyroiditis, appendicitis, diverticulitis, sarcoidosis, dermatoses, vasculitis, lupus and scleroderma or combinations thereof. The RSI, RDTI, RSTI tests or combinations thereof in one or more leukocyte subsets, and the data generated may be used to assess the status of a subject as related to inflammatory disease states. With respect to other cells, the methodology disclosed in this example may be used to set up a range parameter of between 1 and 10 or some other suitable range parameter and/or evaluation tool for the RSI, RDTI and/or RSTI. Non-limiting examples of other cells that this may be done for include: CD8 lymphocyte, CD15 leukocytes, neutrophils, CD19 B cells, and CD14 monocyte and so on. Other methodologies may also be used for setting up a range parameter. For example, for cell speed, a fixed speed of 500 μm/min can be defined as 10, while 10 μm/min can be defined as 1 for RSI, so that RSI values for individual subjects can be then determined. In addition, for straightness, straightness values of 1 and 0.1 can be defined as 10 and 1 for RSTI, respectively.

### Example 4: Mn²⁺ Induced Activation Potential of α4 Integrin

The present example is directed to showing semi-quantitative assessment tools that may be used in certain exemplary embodiments. Mn²⁺ is an integrin activator, that enhances the activity of leukocyte membrane α4 integrin, including α41 and α4β7 integrins. The difference of leukocyte ability to bind to α4 integrin ligands in the presence and absence of Mn²⁺ may then be used to define the “activation potential” of α4 integrin, showing how much α4 integrin activation may be induced by Mn²⁺.

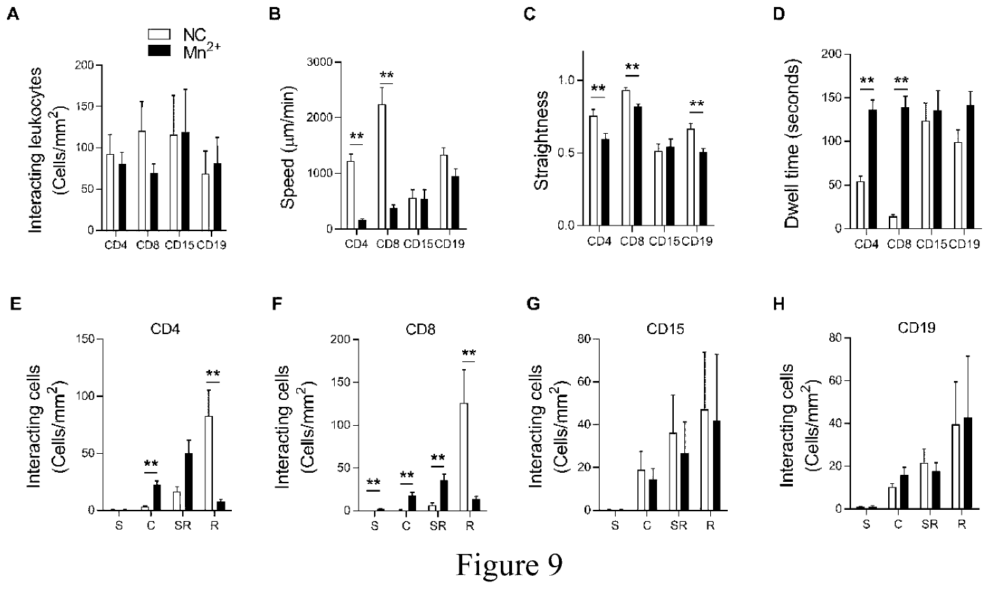

As shown in FIG. 1F and Example 1, the ratio of the average speed between untreated and Mn²⁺ treated cells on VCAM-1 substrate may be defined as “Speed Activation Potential Ratio (SAPR)”, which may then be used to semi-quantitatively assess the activation potential of α4β1 integrin. For example, the average speed of CD4 cells is 163.9 and 23.1 μm/min in the absence and presence of Mn²⁺, respectively, for a particular subject's tested whole blood. Thus, the SAPR value for the CD4 cells of this subject is 163.9/23.1=7.1. In this case, the lower the SAPR value is, the less is the activation potential, and the higher portion of α4β1 integrin is in an activated form. Similarly, the same formula may be used to determine the SAPR values of α4β7 integrin using data in FIG. 9, Example 10.

As shown in FIG. 1G, the ratio of the average dwell time between untreated and Mn²⁺ treated cells on VCAM-1 substrate may be defined as “Dwell Time Activation Potential Ratio (DTAPR)”, which may also be used to semi-quantitatively assess the activation potential of α4β1 integrin. For example, the average dwell time of CD4 cells is 1.43 and 4.03 minutes in the absence and presence of Mn²⁺, respectively, for a particular subject's tested whole blood. Thus, the DTAPR value for the CD4 cells of this subject's blood will be 1.43/4.03=0.355. In this case, the higher the DTAPR value is, the less is the activation potential, and the higher portion of α4β1 integrin is in an activated form. Similarly, the same formula may be used to determine the DTAPR values of α4β7 integrin using data in FIG. 9, Example 10.

As shown in FIG. 1H, the ratio of the average straightness between untreated and Mn²⁺ treated cells on VCAM-1 substrate may be defined as “Straightness Activation Potential Ratio (STAPR)”, which may also be used to semi-quantitatively assess the activation potential of α4β1 integrin. For example, the average straightness of CD4 cells is 0.40 and 0.12 in the absence and presence of Mn²⁺, respectively, for a particular subject's tested whole blood. Thus, the STAPR value for the CD4 cells of this subject's blood will be 0.40/0.12=3.33. In this case, the less the STAPR value is, the less is the activation potential, and the higher portion of α4β1 integrin is in an activated form. Similarly, the same formula may be used to determine the DTAPR values of α4β7 integrin using data in FIG. 9, Example 10.

The Mn²⁺ induced activation potential of α4β1 and/or α4β7 integrin may be used as a semi-quantitative tool to assess the portion of activated α4β1 and/or α4β7 integrin in a subject or subjects, such as patients with multiple sclerosis, Crohn's disease, colitis, atherosclerosis, autoimmune thyroiditis, appendicitis, diverticulitis, sarcoidosis, dermatoses, vasculitis, lupus and scleroderma or combinations thereof. The SAPR, DTAPR. DTAPR tests or combinations thereof in one or more leukocyte subsets, and the data generated may be used to assess the status of a subject as related to inflammatory disease states. The uses of Mn²⁺ induced activation potential ratio in clinical settings are discussed in Examples 21, 22, 24 and 25.

### Example 5: Natalizumab Inhibits VCAM-1 Dependent Leukocyte Recruitment

The present example is directed to detect Natalizumab efficacy using leukocyte adhesive function assay. The protocol set forth in Appendix I was used with the following modifications to perform the experiments:

1. Blood samples were treated with a range of doses of Natalizumab (Biogen, Cambridge Mass.) at room temperature, before being used for LAFA analysis. For the Mn²⁺activated samples, blood was treated with Mn²⁺ for approximately 5 min at room temperature, before being treated with different doses of Natalizumab (e.g. 0.01, 0.03, 0.1, 0.2, 0.3, 1, 3 and 10 μg/ml) for approximately 5 min at room temperature in whole blood.

Natalizumab, a neutralising anti-human α4 integrin antibody, is believed to be one of the most effective treatment for patients with relapsing-remitting multiple sclerosis (RRMS). Natalizumab inhibits α4 integrin ligand binding, leading to a reduction of leukocyte recruitment. In the present example, therefore, the ability of the system, according to exemplary embodiments, to detect the Natalizumab-induced decrease in α4 integrin adhesive function were tested. Blood samples were treated with a range of doses of Natalizumab (0.01, 0.03, 0.1, 0.2, 0.3, 1, 3 and 10 μg/ml) before being used for the flow assay.

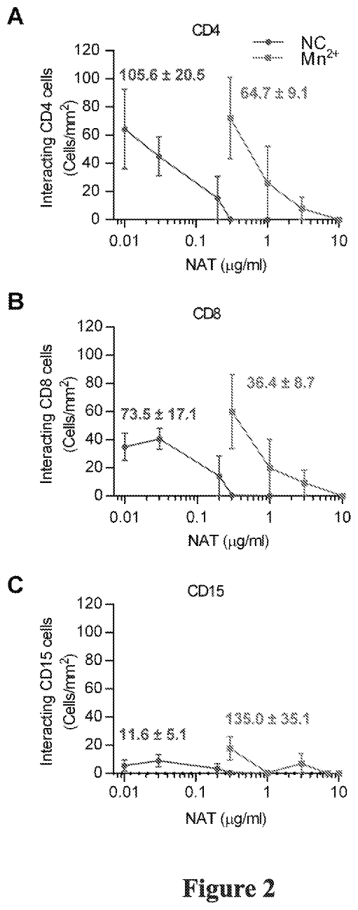

In the absence of Mn²⁺, 0.01 μg/ml of Natalizumab treatments had no effect on the number of CD4 interacting cells (69.8±29.4 cells/mm²), compared to no Natalizumab controls (105.6±20.5 cells/mm², FIG. 2A). On the other hand, this level of Natalizumab caused a ˜50% reduction (p<0.05) in the number of interacting CD8 cells (36.9±10.1 cells/mm²), compared to no Natalizumab controls (73.5±17.1 cells/mm², FIG. 2B). The increase of Natalizumab dosage gradually reduced the number of CD4 and CD8 interacting cells. At 0.3 μg/ml. Natalizumab completely inhibited VCAM-1 dependent recruitment of CD4 and CD8 cells (FIGS. 2A and 2B).

In Mn²⁺ treated blood, however, 0.3 μg/ml Natalizumab had no effects on the recruitment of CD4 and CD8 cells, compared to no Natalizumab controls (FIGS. 2A and 2B). The minimum of Natalizumab dose required to completely inhibit the recruitment of Mn²⁺ activated CD4 and CD8 cells were determined to be 10 μg/ml (FIG. 2A). In addition, Mn²⁺ treatments induced an over 15 fold increase in Natalizumab IC50 values in both CD4 and CD8 cells (FIG. 2D), clearly indicating Mn²⁺ induced activation on α4 integrin adhesive functions. Interestingly, Mn²⁺ activated CD8 cells showed a minor but significant (p<0.05) increase in IC50 values compared to CD4 cells, suggesting activated CD8 cells are marginally more resistant to Natalizumab than activated CD4 cells.

In the absence of Mn²⁺, only a small number of interacting CD15 cells was observed (FIG. 1B). When treated with Natalizumab, the number of interacting CD15 cells remained low and 0.3 μg/ml of Natalizumab led to a complete elimination of interacting CD15 cells (FIG. 2C). In Mn²⁺ treated blood, interestingly, 0.3 μg/ml of Natalizumab caused an 8-fold decrease in the number of CD15 cells to 18.7±8.6 cells/mm² (p<0.01), compared with no Natalizumab controls (135.0±35.1 cells/mm², FIG. 2C). In addition, a complete inhibition of CD15 cells recruitment was achieved at 7 μg/ml of Natalizumab (FIG. 2C). Consistently, IC50 values of Mn²⁺ treated CD15 cells arc significantly lower than those of CD4 and CD8 cells (p<0.05, FIG. 2D). These results illustrate that Mn²⁺-treated CD15 cells are more sensitive to Natalizumab treatments, compared to CD4 and CD8 cells.

These results clearly show the ability of LAFA exemplary embodiments to accurately assess Natalizumab efficacy in vitro. Thus, LAFA exemplary embodiments may be used to assist the pharmacokinetics (PK) studies of certain drugs. Traditionally. PK studies for most drugs are conducted based on the serum levels of the drugs or related drug metabolites. However, quantity does not necessarily translate into functionality. Certain embodiments herein are directed to techniques that may be used to assess the primary function of Natalizumab, without regard, or substantially without regard to drug concentrations in the blood of the subject, allowing a more accurate assessment of drug effectiveness. As a result, the techniques discussed herein may be a useful tool to improve the accuracy of PK studies for new/other drugs.

### Example 6: Accuracy and/or Sensitive Advantage of LAFA

The present example is directed to illustrate the accuracy and/or sensitivity advantage of LAFA exemplary embodiments to assess Natalizumab efficacy, comparing to conventional ligand occupancy assay, according to certain exemplary embodiments.

To perform the ligand occupancy assay, 100 μl whole blood were treated with or without 5 mM MnCl₂ for approximately 10 min at room temperature. A range of doses of Natalizumab (e.g. 0.001, 0.01, 0.1, 1, 10, 100 and 300 gag/ml) were then added to the blood, and incubated for approximately 5 min at room temperature. 2 μl of anti-human CD4-Alexa488 antibody was also added to detect CD4 positive T lymphocytes. After this, red blood cells were lysed using 5 ml of red blood lysis buffer (NH₄Cl) to remove the red blood cells. After being washed with PBS buffer for 3 times, a PE conjugated anti-human IgG secondary antibody (1:500 diluted) was added to the leukocytes and incubated for approximately 20 min at room temperature in the dark. Cells were then washed with PBS buffer 3 times before being used for the flow cytometric assay. Alexa488 was used to identify CD4 positive T lymphocytes, and the PE positivity and mean fluorescence intensity were used to assess the Natalizumab binding capability to α4 integrin.

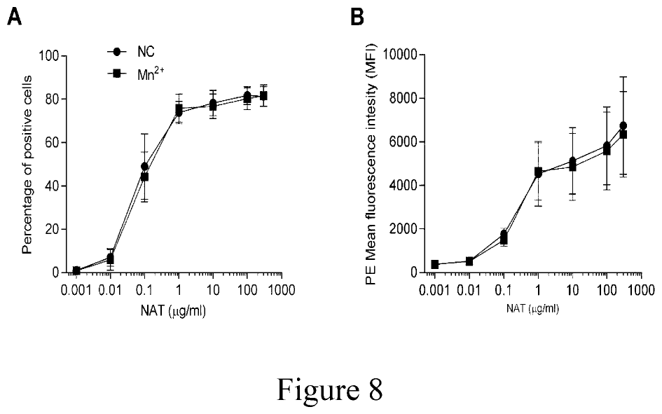

In control (untreated) blood, 0.001 μg/ml of Natalizumab did not lead to a PE positive CD4 lymphocytes (FIG. 8A, circles), showing no Natalizumab binding to α4 integrin was induced by this dose of Natalizumab. On the other hand, the percentage of PE positive CD4 lymphocytes was gradually increased with the increase of Natalizumab doses, and reached a plateau of 73.9% at 1l g/ml (FIG. 8A). Additionally, comparing to 1 μg/ml, no further significant increase in the percentage of PE positive CD4 lymphocytes was detected at higher doses of Natalizumab (FIG. 8A), showing the binding of Natalizumab to α4 integrin was saturated at 1 μg/ml. Consistently, the PE MFI of CD4 lymphocytes was gradually increased with the increase of Natalizumab doses, and reached a plateau at 1 μg/ml (FIG. 8B, circles), showing that Natalizumab occupancy on α4 integrin was saturate at 1 μg/ml of Natalizumab.

In Mn²⁺ activated blood, the α4 integrin occupancy by Natalizumab was almost identical to what was observed in untreated blood. As shown in FIGS. 8A and 9B (squares), both the percentage of PE positive CD4 lymphocytes and PE MFI was saturated at 1 μg/ml of Natalizumab, showing that Mn² activation had no effects on the binding of Natalizumab to α4 integrin.

Together, these findings show that the conventional ligand occupancy assay failed to detect the higher requirement of Natalizumab to completely inhibit α4 integrin function in Mn²⁺ activated cells, comparing to untreated controls. On the other hand, the different doses required to completely inhibit α4 integrin functions between Mn²⁺ treated and untreated cells were clearly detectable using LAFA analysis (FIG. 2A). Thus, these data shows sensitivity and accuracy advantages of LAFA exemplary embodiments over the conventional ligand occupancy assay. Thus, the use of LAFA to accurately determine drug efficacy may be applied to other drugs, including but not limited to PTG-100 and Bio-1211.

### Example 7: Model to Reduce the Risk of PML

The present example is directed to use leukocyte adhesive function assay to optimise Natalizumab treatment regimens and, therefore, reduce the risk of drug-induced side effect, including Progressive multifocal leukoencephalopathy (PML).

Natalizumab suppresses the function of the immune system and, therefore, controls disease progression, but it also put patients at the risk of side effects, such as PML, a rare and frequently fatal disease caused by the infection of John Cunningham virus (JCV). Approximately 50% of multiple sclerosis patients worldwide are carrying JCV, and are at risk of PML when on Natalizumab therapy.

As shown in FIG. 2, LAFA may be used to accurately monitor Natalizumab efficacy and, thus, may be used to determine the need for drug redosing. For example, if at the maximal efficacy of Natalizumab, no cell interaction will be detected by LAFA, indicating no need for drug redosing.

However, once the drug efficacy reduces to below maximal efficacy (e.g. 100%), cell interaction will gradually become detectable by LAFA exemplary embodiments. The restoration of cell interaction indicates a reconstitution of immune response, which may be beneficial for reducing the risk of PML. For example, after the drug infusion, Natalizumab saturation level will be gradually reduced below maximal efficacy to a point (e.g. 95%, 90%, 80%, 70%, 60%, 50%, 40%, 30%, 20% or 10%) where a full drug efficacy may be maintained, which may be defined as Drug Redosing Window (FIG. 26). The reduction of drug saturation to below maximal efficacy may lead to a reconstitution of leukocyte recruitment and immune response, which could allow the immune system to restore the ability to respond to and eliminate JCV infection, leading to a reduced risk of PML.

LAFA exemplary embodiments may be used as a tool to accurately determine the drug redosing window on a subject by subject basis, allowing the necessary reconstitution of immune response without compromising the drug efficacy. If a certain period (e.g. 1, 2, 3, 4, 5, 6 or 7 days) of the drug redoing window is allowed for each, a substantial portion of, or a portion of drug dosing cycle, the risk of PML in patients on Natalizumab therapy may be effectively reduced. Patients on extended dosing interval (up to 5, 6, 7 or 8 weeks) are less likely to have PML that standard dosing interval patients (4 weeks).

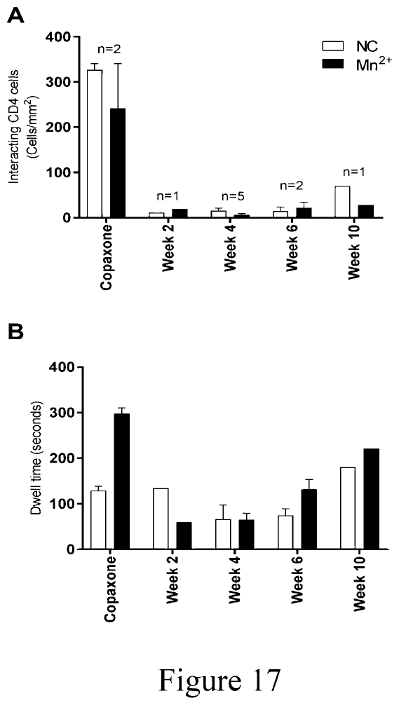

As shown in Tables 1 and 5, however, the Natalizumab sensitivity may vary dramatically between individual subjects, suggesting that a fixed dosing interval may not work or work as effectively for some subjects. This is supported by the data in FIG. 17, showing that different levels of Natalizumab efficacy was detected in a group of MS patients 4 weeks post drug infusion. Thus, LAFA exemplary embodiments may be used to determine the drug redosing window in individual subjects, leading to a more effective personalised treatment regimens for individual patients and a reduction in the risk of PML because the dosing interval may be extended in a subject.

### Example 8: Low (10 μg/ml) and High Dose (300 μg/ml) Natalizumab Inhibits Leukocyte Firm Adhesion on TNFα Activated-HUVEC

The present example is directed to confirm that low and high dose of Natalizumab have similar effects on leukocyte adhesive functions. LAFA exemplary embodiments were used to show there is no additional therapeutic benefit, or substantially no additional therapeutic benefit, of high doses of Natalizumab, comparing to low doses.

The recruitment of leukocytes to the inflamed tissue involves a series of interactions between leukocytes and endothelial cells, which is mediated by multiple adhesion molecules and their correspondent ligands. Natalizumab inhibition of α4 integrin abolished VCAM-1 dependent leukocyte recruitment is shown herein; therefore, the effects of α4 integrin blockage on leukocyte interaction with endothelial cells were assessed in the next set of experiments. Thus, to realistically recapitulate in vivo microcirculation, the effects of Natalizumab on the interaction between leukocytes and tumor necrosis factor α (TNFα) activated human primary HUVEC under flow conditions were studied next.

The original pharmacokinetics (PK) study in human suggested that the average of Natalizumab serum level may reach 110±52 μg/ml right after the drug infusion. Thus, a high dose of Natalizumab (300 μg/ml) was chosen to ensure the saturation of the drug efficacy. The mean Natalizumab serum level drops to ˜10 μg/ml at day 28 after the initial infusion, at which Natalizumab re-dosing is usually recommended, as disclosed in Rudick, R. A., and A. Sandrock. 2004, “Natalizumab: alpha 4-integrin antagonist selective adhesion molecule inhibitors for MS. Expert review of neurotherapeutics” 4: 571-580 which is hereby incorporated by reference in its entirety. Therefore, a low Natalizumab dose (10 μg/ml) were also included herein in the present example.

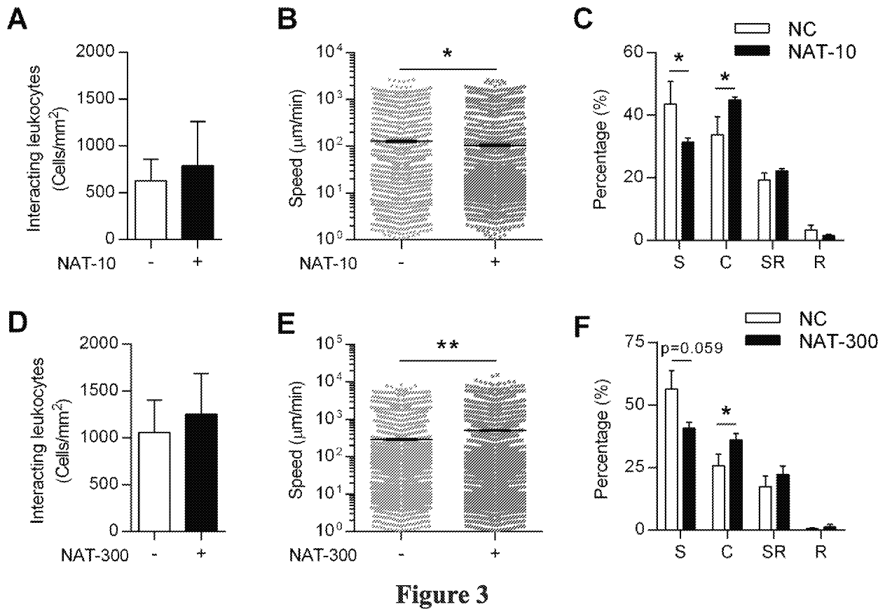

Leukocytes were labelled with Hoechst33342 only in unprocessed human whole blood and the high frame rate of 2 frames per sec was used, allowing the capture of the types of interacting cells. Compared to Natalizumab controls, no effects on the number of interacting leukocytes were detected, when treated with either low (10 μg/ml) or high dose (300 μg/ml) of Natalizumab (FIGS. 3A and 3D). Despite the fact that the average leukocyte migration speed was slightly decreased (p<0.05) by low dose Natalizumab (FIG. 3B), high dose of Natalizumab significantly increased (p<0.01) the cell speed, compared to no Natalizumab controls (FIG. 3E). Further cell kinetic analysis showed that 10 μg/ml of Natalizumab significantly reduced the percentage of static cells (p<0.05) from 43.6±7.3% (no Natalizumab controls) to 31.4±1.4%, while the portion of crawling cells was significantly increased (p<0.05) (FIG. 3C). Similarly, an almost identical effect on the percentage of static and crawling cells was also detected in high dose Natalizumab treated cells (FIG. 3F). These findings show that both low and high dose of Natalizumab are able to suppress leukocyte firm adhesion on human endothelial cells in a similar manner.

Low and high dose Natalizumab alter CD4 and CD15, but not CD8, cell migratory behaviours on TNFα activated-HUVEC.

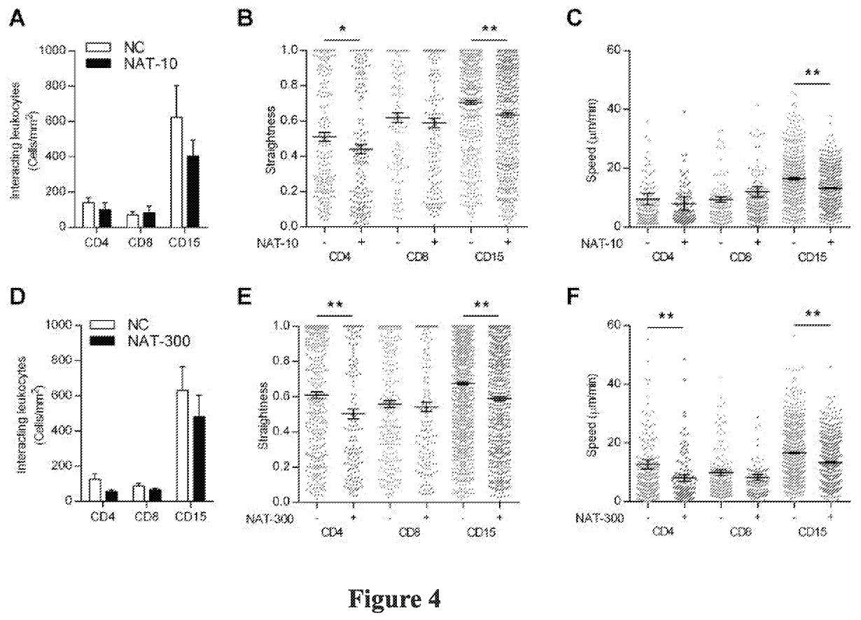

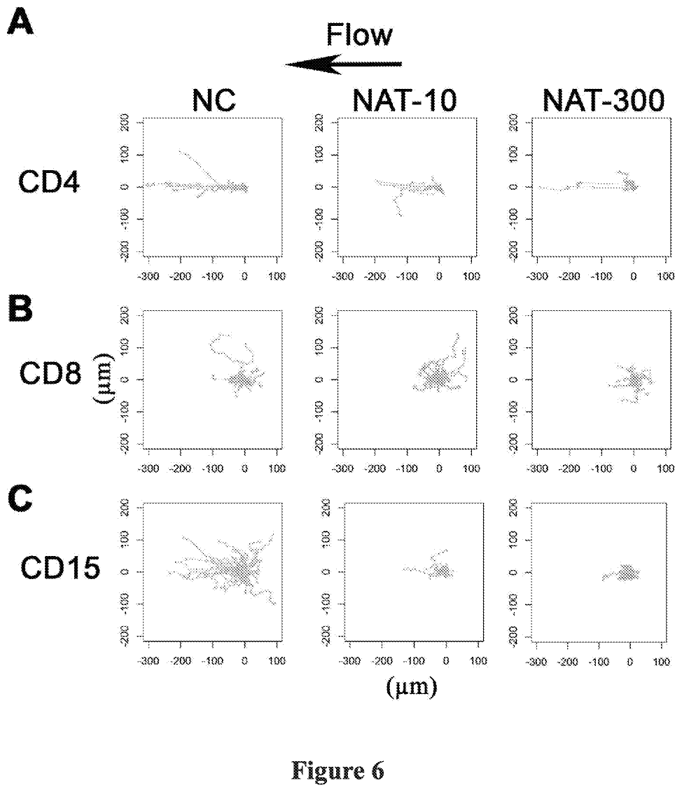

To further characterise effects of the low and high dose Natalizumab on specific leukocyte subsets, fluorescently labelled anti-CD4, CD8 and CD15 antibodies were added to blood samples. Three dimensional (3D) image stacks were acquired at 1 frame every 30 seconds, providing 3D data sets of slow moving cells (static and crawling cells) over time. As shown in FIGS. 4A and 4D, neither 10 μg/ml nor 300 μg/ml of Natalizumab affected the number of interacting CD4, CD8 and CD15 cells on TNFα-activated HUVEC. Compared to no Natalizumab controls, both low and high dose of Natalizumab significantly reduced the straightness of CD4 (p<0.05) and CD15 (p<0.01) cells, whereas no such effect on CD8 cells were detected (FIGS. 4B and 4E). Additionally, compared to no Natalizumab controls, the migration speed of CD15 cells were significantly reduced by both low and high dose of Natalizumab, while high dose of Natalizumab also caused a decrease in the speed of CD4 cells (FIGS. 4C and 4F). These findings illustrate an ability of Natalizumab to inhibit the motility of these cells. On the other hand, no such effect on cell speed by either low or high dose of Natalizumab were detected in CD8 cells (FIGS. 4C and 4F). Consistently, the inhibitory effect of Natalizumab on CD4 and CD15 cell motility is also supported by the common origin graphs (FIG. 6). Together, these results illustrate that the low (10 μg/ml) and high (300 μg/ml) dose of Natalizumab have almost identical effects on leukocyte migratory behaviours on TNFα-activated HUVEC.

### Example 9A: Natalizumab Effectiveness Test

The standard dose of Natalizumab is 300 mg per patient per infusion, generally given once every 4 weeks. The approved dosing regimen for Natalizumab is based on serum concentrations of the drug, assuming a similar efficacy and metabolism in a highly heterogeneous patient population. However, it has been suggested that extended interval dosing (EID) up to 8 weeks may not only maintain the same treatment efficacy, but also reduce the risk of progressive multifocal leukoencephalopathy (PML), a fatal side effect of Natalizumab therapy. On the other hand, determining the optimal and/or personalised dosing intervals to ensure drug effectiveness in individual patients is useful for such improvements. The present disclosure provides such technology and capability. As described herein, certain exemplary embodiments arc directed to new technology that has been developed, allowing a fast and/or accurate assessment of Natalizumab effectiveness, regardless of serum drug concentrations.

A dose dependent reduction in the number of interacting cells were observed when treated with Natalizumab (FIGS. 2A-2C), providing a quantitative approach to assess Natalizumab effectiveness. The absence of interacting cells may be used as an indicator for positivity of Natalizumab effectiveness. In addition, the results showed that the IC50 values between individual blood donors may vary up to 10 times (Table 1), illustrating there is a significant difference in drug sensitivity between different individuals.

TABLE 1—IC50 values between individual blood donors. Blood was treated with or without 5 mM Mn²⁺, before being treated with a series of doses of Natalizumab (0.001, 0.003, 0.01, 0.03, 0.1, 0.3, 1, 3 and 10 μg/ml). Subsequently, blood samples were analysed in VCAM-1 coated microfluidic channels and the number of CD4, CD8 and CD15 interacting cells were determined. The half maximal inhibitory concentration (IC50) values of Natalizumab was then calculated. *, p<0.05, compared to the values of CD4 and CD8 cells; #, p<0.05, compared to the values of CD4 cells.

TABLE 1

| | | | | | | |
|---|---|---|---|---|---|---|
| IC50 | | | | | | |
| (ug/ml) | Donor 1 | Donor 2 | Donor 3 | Donor 4 | Donor 5 | Average |

NC

| | | | | | | |
|---|---|---|---|---|---|---|
| CD4 | 0.006 | 0.022 | 0.014 | 0.064 | 0.046 | 0.030 ± 0.01 |
| CD8 | 0.007 | 0.041 | 0.013 | 0.047 | 0.119 | 0.024 ± 0.01 |
| CD15 | 0.071 | 0.328 | 0.009 | 0.002 | 0.002 | 0.0824 ± 0.06 |

Mn²⁺

| | | | | | | |
|---|---|---|---|---|---|---|
| CD4 | 0.162 | 0.12 | 0.349 | 1.705 | 0.15 | 0.497 ± 0.30 |
| CD8 | 0.603 | 0.181 | 0.859 | 1.818 | 0.429 | 0.788 ± 0.28# |
| CD15 | 0.075 | 0.106 | 0.013 | 0.237 | 0.009 | 0.088 ± 0.04* |

Thus, to ensure patient safety, Natalizumab effectiveness tests typically is performed in individual patients. It was also noted that the minimal Natalizumab concentration required to completely block α4 integrin functions were found to be much lower than what is currently recommended. These results illustrate that Natalizumab may retain its effectiveness at much lower level than what was previously recognised.

These findings may be developed into a new blood test, performed at various time points post-Natalizumab infusion. Accordingly, the test results may be used to determine the need of Natalizumab re-dosing. This blood test may be conducted in individual patients to ensure drug effectiveness, facilitating the development of optimal and/or personalised treatment regimen for individual patients.

### Example 9B: Natalizumab Sensitivity Variability in a Single Healthy Subject from Multiple Tests

The present example is directed to assess Natalizumab sensitivity in a single subject from multiple tests. Blood samples were collected from a single healthy subject every 1-2 weeks for 5 times. The samples were then used for the leukocyte adhesive function assay analysis using VCAM-1 as adhesive substrate to determine IC50 value for each test, according to the protocol as described in Example 5. The IC50 value is exemplified using the Natalizumab inhibitory effects on the number of CD4 interacting cells.

As shown in Table 2, the IC50 value from test 2 (0.2624 μg/ml) is almost 6 time higher than the value from test 4 (0.047 μg/ml), showing that the Natalizumab sensitivity in a single healthy subject may vary dramatically over time. These results show a good sensitivity of LAFA exemplary embodiments to determine the subtle differences of Natalizumab sensitive in the same subject at different time points. These differences also demonstrated the need to monitor Natalizumab efficacy and determine the drug redosing window (detailed in Example 7) in individual patients on regular basis.

TABLE 2
In table 2 the IC50 values in a single healthy subject from multiple tests
are provided.

| | Test | | | | |
|---|---|---|---|---|---|
| | Test 1 | Test 2 | Test 3 | Test 4 | Test5 |
| IC50 | 0.0949 | 0.2624 | 0.05709 | 0.047 | 0.1932 |
| (μg/ml) | | | | | |

Blood samples were collected from a single healthy subject every 1-2 weeks for 5 times. The samples were then used for the leukocyte adhesive function assay, according to exemplary embodiments, using VCAM-1 as adhesive substrate to determine IC50 value for each test, according to the protocol in Appendix A. The IC50 value is exemplified using the Natalizumab inhibitory effects on the number of CD4 interacting cells.

### Example 10: Mn Effects on Leukocyte Migration Profile on MAdCAM-1 Substrate

The present example is directed to assess the ability of leukocyte to interact with endothelial MAdCAM-1. Based on the protocol as set forth in appendix A, the following modifications were made to perform the experiments:

1. Human MAdCAM-1 protein (R&D, Catalogue number: 6056-MC) was used to pre-coat the microfluidic channel at a concentration of 5 μg/ml.

2. The following antibodies were added to human whole blood sample to identify specific leukocyte subsets:
CD4-Alexa488
CD8-PE
CD15-APC
CD19-BV510

To characterise the cell migratory behaviours, a range of cell kinetic parameters were utilised to assess the ligand binding activity of α4β7 integrin. As shown in FIG. 9A, Mn²⁺ treatments did not affect the number of interacting CD4, CD8, CD15 and CD19 cells on MAdCAM-1 substrate, comparing to untreated controls. However, the average speed of CD4 and CD8 cells were significantly (p<0.01) reduced to 167.2±21.4 and 375.0±64.1 μm/min respectively, comparing untreated controls (CD4, 1,227.3±115.0 and CD8, 2,248.8±293.2), whereas no such reduction was detected in CD15 and CD19 cells (FIG. 9B). These results show that Mn²⁺ may enhance the ability of α4β7 integrin to bind to MAdCAM-1 substrate, which may lead to a stronger cell-MAdCAM-1 interaction and a lower speed of interacting leukocytes.

Based on their mean speed of migration, total interacting cells were divided into static (<5 μm/min), crawling (5-20 μm/min), slow rolling (20-300 μm/min) and rolling cells (300-6000 iμm/min), as described in detail in Example 1. As shown in FIGS. 9E and 9F, Mn²⁺ significantly (p<0.01) increased the number of static, crawling, and slow rolling CD4 and CD8 cells, whereas the number of fast rolling CD4 and CD8 cells was significantly reduced, comparing to no Mn²⁺ controls (FIGS. 9E and 9F). These results are consistent with the Mn²⁺ effects on cell speed, as shown in FIG. 9B. No obvious Mn²⁺ effect on interacting CD15 and CD19 cells was detected (FIGS. 9G and 9H).

Additionally, the straightness of interacting CD4, CD8 and CD19 cell was significantly (p<0.01) reduced by Mn²⁺ treatments, comparing to the untreated controls, showing a stronger Mn²⁺-induced cell and substrate interaction. Similarly, Mn²⁺ also significantly (p<0.01) increased the dwell time of CD4 and CD8 cells to 135.8±11.4 and 138.8±12.9 seconds respectively, comparing to untreated controls (CD4, 54.1±6.0 and CD8, 14.2±1.8 seconds) (FIG. 9D).

Together, these findings demonstrated the ability of Mn²⁺ to enhance α4β7 integrin activity and lead a stronger cell-MAdCAM-1 interaction. These results also show an ability of LAFA exemplary embodiments to accurately and/or quantitatively assess α4β7 integrin activity using whole blood sample in vitro.

In addition, the uses of LAFA may be extended to assess adhesive functions of other leukocyte adhesion molecules. In the present study the interaction of leukocyte α4β1 and α4β7 integrin with endothelial VCAM-1 and MAdCAM-1 was investigated, and the ability to quantitatively detect the activation and inhibition of α4 integrin has been clearly demonstrated by the data. The techniques described herein may be easily extended to other leukocyte adhesion molecules well as other binding molecules of leukocytes, which are also involved in the pathogenesis of many other human diseases (e.g. Examples 16 and 21). For example, the effects of leukocyte expressed chemokine receptors on leukocyte adhesive functions may also be studied using the same techniques, as detailed in Examples 17 and 28. As a result, the applications of LAFA exemplary embodiments may be significantly extended into many other drugs and human diseases.

Potential applications include:
Patient grouping/stratification for other existing drugs;
Identification of new treatment targets in other human diseases; and
Extending applications for other existing drugs

The candidates of these leukocyte adhesion molecules, their specific ligands, related diseases, drugs targeting these molecules and drug manufacturers include those shown in Table 3 below.

TABLE 3
Other leukocyte adhesion molecule candidates, ligands, diseases and drugs
of interest.

| Leukocyte Ad | | | | |
|---|---|---|---|---|
| Mols | Ligands | Diseases | Drugs | Company |
| α4β1 integrin | VCAM-1 | Multiple sclerosis, Crohn's | Natalizumab | Biogen |
| | | disease | | |
| α4β7 integrin | MAdCAM-1 | Crohn's disease, Ulcerative | Vedolizumab | Millennium/ |
| | | colitis | | Genentech |
| α4β7 integrin | MAdCAM-1 | Crohn's disease, Ulcerative | PTG-100 | Protagonist |
| | | colitis | | |
| A4β1 integrin | VCAM-1 | Multiple sclerosis | Bio-1211 | Biogen |
| β7 integrin | | Ulceractive colitis | Etrolizumab | Genentech |
| CD11a | ICAM-1 | Asthma, Psoriasis, | Efalizumab | Genentech/ |
| (α_{L} integrin) | | Rheumatoid arthritis | | Xoma |
| CD11a | ICAM-1 | Transplant | Odulimomab | IMTIX |
| (α_{L} integrin) | | | | |
| CD11b | ICAM-1 | Stroke | UK279, 276 | Pfizer |
| (α_{M} integrin) | | | | |
| β2 integrin | Multiple | Myocardial infarction, | Erlizumab | Genentech |
| | | Traumatic shock | | |
| β2 integrin | Multiple | MS, Myocardial infarction, | Roverlizumab | ICOS |
| | | Traumatic shock, Stroke | | |

Table 3: List of drugs have been or are being developed.
Note:
Underlined drugs mean these drugs are currently on the market.

### Example 11: Semi-Quantitative Assessment of Basal Inflammatory Status of α4β7 Integrin

The present example is directed to showing semi-quantitative assessment tools that may be used in certain exemplary embodiments.

As shown in FIGS. 9B to 9D, a range parameter may be used to characterise the basal inflammatory status of α4β7 integrin. In FIG. 9B, if the average white blood cell speed of control and Mn²⁺ treated leukocytes are arbitrarily set to be 10 and 1 (also referred to as Relative Speed Index (RSI)) respectively, the RSI value offers a semi-quantitative tool to assess α4β7 integrin adhesive functions in an individual subject and/or one or more subjects. For example, in CD4 cells, the average white blood cell speed is 1,127.3 and 167.2 μm/min in the absence and the presence of Mn²⁺, which may then be defined as 10 and 1 respectively, the RSI values for CD4 cells. If the CD4 cell RSI value of a subject falls between 10 and 1, the closer the RSI is to 1, the more active is the α4β7 integrin, and the higher is the basal cell inflammatory status.

Similarly, in FIG. 9C, if the average dwell time of control and Mn²⁺ treated leukocytes are arbitrarily set to be 1 and 10 (also referred to as Relative Dwell Time Index (RDTI)) respectively, the RDTI value will also offer a semi-quantitative tool to assess α4β7 integrin adhesive functions. For example, in CD4 cells, the average white blood cell dwell time is 54.1 and 135.9 seconds in the absence and the presence of Mn²⁺, which may then be defined as 10 and 1 respectively, the RDTI values for CD4 cells. In this case, the closer the RDTI is to 10, the more active is α4β7 integrin.

In FIG. 9D, if the average straightness values of control and Mn² treated leukocytes are arbitrarily set to be 10 and 1 (also referred to as Relative Straightness Index (RSTI)) respectively, the RSTI offers a semi-quantitative approach to assess α4β7 integrin adhesive functions. For example, in CD4 cells, the average white blood cell straightness is 0.76 and 0.60 in the absence and the presence of Mn², which may then be defined as 10 and 1 respectively, the RSTI values for CD4 cells. In this case, the closer the RSTI is to 1, the more active is α4β7 integrin.

The basal inflammatory level of α4β7 integrin may be used as a semi-quantitative tool to assess basal level of α4β7 integrin activation and/or basal inflammation status of a subject or subjects, such as patients with multiple sclerosis, Crohn's disease, colitis, atherosclerosis, autoimmune thyroiditis, appendicitis, diverticulitis, sarcoidosis, dermatoses, vasculitis, lupus and scleroderma or combinations thereof. The RSI, RDTI, RSTI tests or combinations thereof in one or more leukocyte subsets, and the data generated may be used to assess the status of a subject as related to inflammatory disease states.

### Example 12: The Detection of Vedolizumab Efficacy

The present example is directed to detect Vedolizumab efficacy using leukocyte adhesive function assay exemplary embodiments. Based on the protocol described in Example 10, the following modifications were made to perform the experiments:

1. Blood samples were treated with a range of doses of Vedolizumab (Takeda) at room temperature for approximately 5 minutes, before being used for LAFA exemplary embodiments. For the Mn²⁺ activated samples, blood was treated with Mn²⁺ for approximately 5 minutes at room temperature, before being treated with different doses of Vedolizumab for approximately 5 minutes at room temperature.

Vedolizumab, a neutralising anti-human α4β7 integrin antibody, is used for the treatments of patients with inflammatory bowel diseases, including Crohn's disease and colitis. Vedolizumab inhibits α4β7 integrin adhesive function, leading to a reduction of leukocyte recruitment. In the present study, therefore, the ability of a system according to certain embodiments was used to detect the Vedolizumab-inhibited α4β7 integrin adhesive functions using MAdCAM-1 as an adhesive substrate. Whole blood samples were treated with a range of doses of Vedolizumab in whole blood before being used for the leukocyte adhesive function assay exemplary embodiments.

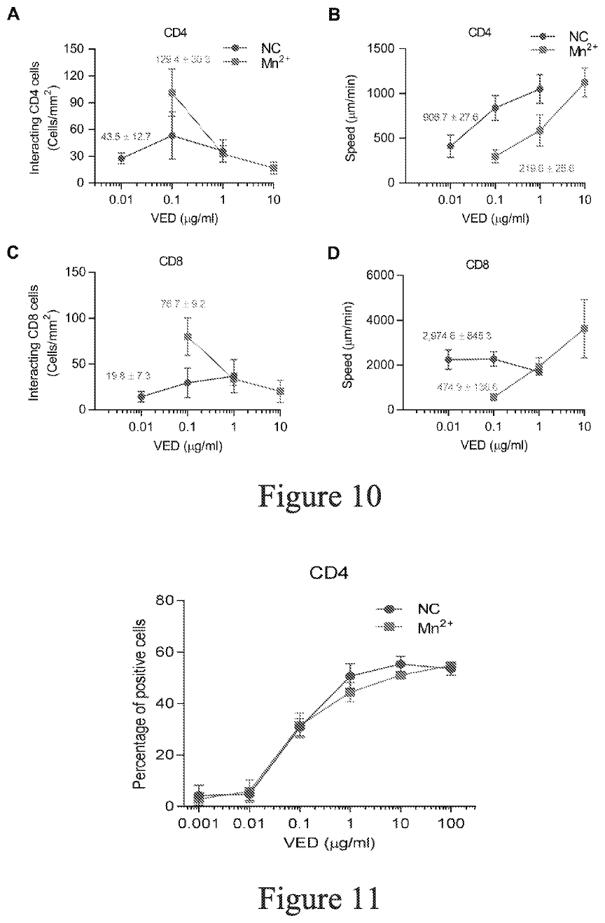

In the absence of Mn²⁺, no obvious Vedolizumab (range from 0.01 to 1 μg/ml) effects on the number of interacting CD4 and CD8 cells was detected (FIGS. 10A and 10C). Comparing to no Vedolizumab controls (906.7±27.6 μm/min), 0.01 μg/ml of Vedolizumab treatments led to a reduction in the average cell speed to 411.5±85.9 μm/min in CD4 cells (FIG. 10B). Similarly, the cell speed of CD8 cells was also decreased from 2,974.6±845.3 to 2,248.6±373.2 μm/min by the same dose of Vedolizumab (FIG. 10D). These results show 0.01 μg/ml of Vedolizumab slightly enhances the interaction between CD4 and CD8 cells with MAdCAM-1 substrate. The reduced speed of interacting CD4 cells was gradually increased and reached to the same level as no Vedolizumab controls at 1 μg/ml, showing that the high doses (>=0.1 μg/ml) of Vedolizumab has inhibitory effects on the interaction between CD4 cells and MAdCAM-1 substrate (FIG. 10B). On the other hand, the increase of Vedolizumab has no effects on the speed of CD8 cells (FIG. 10D).

In Mn²⁺ activated cells, however, the increase of Vedolizumab (from 0.1 to 10 μg/ml) resulted in a gradually decreased number of interacting CD4 cells from 129.4±30.3 cells/mm² (no Vedolizumab control) to 16.9±5.8 cells/mm² (10 μg/ml of Vedolizumab), indicating an inhibitory effect of Vedolizumab on CD4 cell recruitment on MAdCAM-1 substrate (FIG. 10A). A similar Vedolizumab inhibitory effect was also observed in Mn²⁺ activated CD8 cells (FIG. 10C). The IC50 value for Mn²⁺ activated CD4 cells in individual donors was calculated as described in Example 5. As shown in Table 4, the IC50 value for donor 4 is almost 2-fold higher than donor 2, showing a good ability of LAFA exemplary embodiments to detect Vedolizumab sensitivity levels in different subjects.

Additionally, the average speed of CD4 cells were gradually increased with the increase of Vedolizumab concentrations and, at 10 μg/ml, the cell speed reached the same level, or substantially the same levels, as no Vedolizumab controls (FIG. 10B). This shows that Vedolizumab inhibits α4β7 integrin functions, leading to a weaken cell interaction with MAdCAM-1 and an increased cell speed. Similarly, the average speed of Mn²⁺ treated CD8 cells were also enhanced with the increase of Vedolizumab doses (FIG. 10D), showing a similar inhibitory effect of Vedolizumab on CD8 cell recruitment on MAdCAM-1 substrate. It was also noted that the Vedolizumab dose response curves are separated between CD4 cells treated with and without Mn²⁺, demonstrating the ability of certain exemplary embodiments to accurately determine different levels of Vedolizumab sensitivity (FIG. 10B).

Together, these results show an ability of LAFA exemplary embodiments to accurately and/or quantitatively assess not only the Vedolizumab efficacy, but also the subtle differences in Vedolizumab sensitivity between different subjects.

TABLE 4
Vedolizumab sensitivity in individual subjects. Whole blood was activated
with Mn²⁺ before being treated with a range doses of Vedolizumab. Blood
was then analysed by LAFA and IC50 values were then determined as
described in Example 5.

| | Subject | | | | |
|---|---|---|---|---|---|
| | Subject 1 | Subject 2 | Subject 3 | Subject 4 | Subject 5 |
| IC50 | 0.6874 | 0.4913 | 0.5507 | 0.952 | 0.4853 |
| (μg/ml) | | | | | |

Additionally, the average speed of CD4 cells were gradually increased with the increase of Vedolizumab concentrations and, at 10 μg/ml, the cell speed reached the same level, or substantially the same levels, as no Vedolizumab controls (FIG. 13B). This shows that Vedolizumab inhibits α4β7 integrin functions, leading to a weaken cell interaction with MAdCAM-1 and an increased cell speed. Similarly, the average speed of Mn²¹ treated CD8 cells were also enhanced with the increase of Vedolizumab doses (FIG. 13D), showing a similar inhibitory effect of Vedolizumab on CD8 cell recruitment on MAdCAM-1 substrate. It was also noted that the Vedolizumab dose response curves are separated between CD4 cells treated with and without Mn²⁺, demonstrating the ability of certain exemplary embodiments to accurately determine different levels of Vedolizumab sensitivity (FIG. 13B).

Together, these results show an ability of LAFA exemplary embodiments to accurately and/or quantitatively assess not only the Vedolizumab efficacy, but also the subtle differences in Vedolizumab sensitivity between different subjects.

### Example 13: The Accuracy and/or Sensitivity Advantage of LAFA, Comparing to Conventional Ligand Occupancy Assay

The present example is directed to show the accuracy and/or sensitivity advantage of LAFA exemplary embodiments, according to certain exemplary embodiments, to assess Vedolizumab efficacy as compared with conventional ligand occupancy assay. Based on the protocol described in Example 6, the following modifications to that protocol were made to perform the experiments:

1. Whole blood was treated with a range of Vedolizumab doses (0.001, 0.01, 0.1, 1, 10 and 100 μg/ml) before being used for the cell preparation processes for FACS analysis.

In the control cells, Vedolizumab doses lower than 0.01 μg/ml did not cause PE positive CD4 cells, showing that no Vedolizumab binding to its ligand (α4β7 integrin) was induced (FIG. 11, circles). The increased doses of Vedolizumab gradually induced the ligand binding, and reached a plateau of 50.7% of PE positive CD4 cells at 1 μg/ml. The higher Vedolizumab doses (up to 100 μg/ml) did not result in a significant increase of ligand binding, showing the Vedolizumab ligand binding was saturated at 1 μg/ml (FIG. 11).

In Mn²⁺ activated cells, the α4β7 integrin occupancy by Vedolizumab was almost identical to what was observed in untreated cells. As shown in FIG. 11 (squares), the percentage of PE positive CD4 lymphocytes was also saturated at 1 μg/ml of Vedolizumab. These results show that the conventional ligand occupancy assay failed to detect the Mn²⁺-induced activation of α4β7 integrin, which was detectable using the LAFA exemplary embodiments as shown in FIG. 10 and Example 12. This example illustrates that LAFA exemplary embodiments are a more accurate and/or more sensitive assay to determine Vedolizumab efficacy in vitro, comparing to conventional ligand occupancy assay.

### Example 14: Vedolizumab Effects on Leukocyte Recruitment on VCAM-1 Substrate

The present example is directed to determine the effects of Vedolizumab on leukocyte recruitment on VCAM-1 substrate. Whole blood samples were treated with low (10 g/ml) and high (100 μg/ml) dose of Vedolizumab before being used for the leukocyte adhesive function assay, as described in Examples 1 and 12.

Vedolizumab is a monoclonal antibody that specifically binds to α4β7 integrin, without cross-reactivity against α4β1 integrin. Vedolizumab is not expected to affect leukocyte recruitment on VCAM-1 substrate. To confirm this, whole blood was treated with two doses of Vedolizumab (10 and 100 μg/ml) before being used for LAFA analysis.

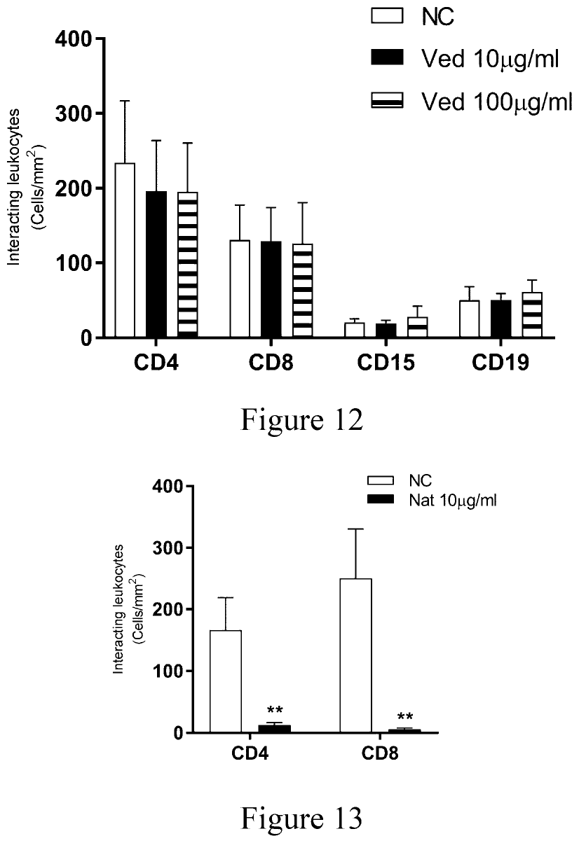

A shown in FIG. 12, no effect of Vedolizumab on the number of interacting CD4, CD8, CD15 and CD19 cell was detected. These results show that Vedolizumab is not able to affect leukocyte recruitment on VCAM-1 substrate, confirming a high septicity of Vedolizumab to its target molecule. These findings also show the ability of LAFA exemplary embodiments to identify potential off-targets of one or more of the following: a drug, a small molecule, an antibody, a peptide and a compound, during the drug development and/or screening processes.

### Example 15: Natalizumab Effects on Leukocyte Recruitment on MAdCAM-1 Substrate

The present example is directed to determine the effects of Natalizumab on leukocyte recruitment on MAdCAM-1 substrate. Whole blood samples were treated with 10 μg/ml of Natalizumab before being used for the leukocyte adhesive function assay, as described in Examples 5 and 12.

Natalizumab is a monoclonal antibody against α4β1 integrin, but is also known to have a cross reactivity to α4β7 integrin. Natalizumab may also inhibit leukocyte recruitment on MAdCAM-1 substrate. To test this, healthy whole blood was treated with or without 10 μg/ml of Natalizumab before being used for LAFA analysis using MAdCAM-1 as substrate, and then the number of interacting cells was determined.

As shown in FIG. 13, 10 μg/ml of Natalizumab almost completed inhibited CD4 and CD8 cell recruitment on MAdCAM-1. These results also show an excellent ability of LAFA exemplary embodiments to identify potential off-targets of one or more of the following: a drug, a small molecule, an antibody, a peptide and a compound, during the drug development and/or screening processes.

### Example 16: Natalizumab and Vedolizumab Effects on Leukocyte Recruitment on P Selectin and E Selectin Substrates

The present example is directed to use leukocyte adhesion assay exemplary embodiments to detect Natalizumab and Vedolizumab effects on leukocyte recruitment on P selectin and E selectin substrates. The protocol set forth in Appendix I was used with the following modifications to perform the experiments:

1. The microfluidic channels were pre-coated with a combination of human P-selectin protein (R&D System, Catalogue number: ADP3) and human E-selectin protein (R&D System, Catalogue number: ADP1), at concentrations of 10 μg/ml and 0.5 μg/ml, respectively.

P selectin and E selectin are two adhesion molecules expressed by blood vessel endothelial cells. P and E selectin selectively bind to their endothelial ligand, P-selectin glycoprotein ligand 1 (PSGL-1), to induce leukocyte tethering and rolling on blood vessel endothelium. As Natalizumab and Vedolizumab specifically binds to leukocyte α4 integrin, either Natalizumab or Vedolizumab will affect the functions of leukocyte expressing PSGL-1. Thus, Natalizumab or Vedolizumab may not have an impact on leukocyte recruitment on P and E selectin (PSGL-1 ligand) substrates.

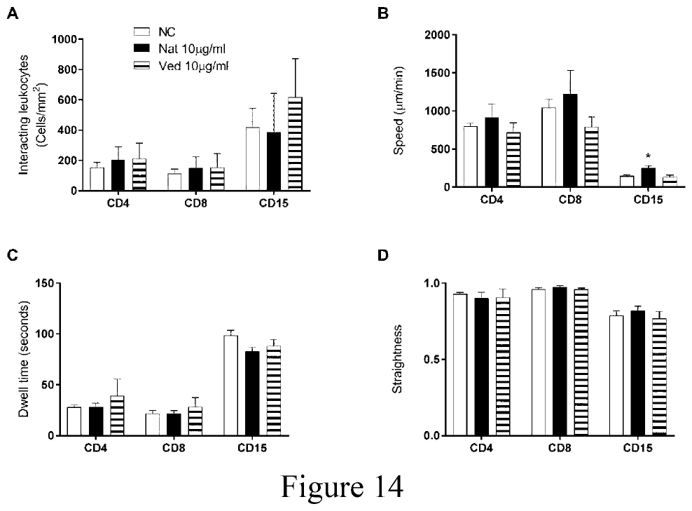

As shown in FIG. 14, except for a slight increase in the speed of CD15 cells by Natalizumab treatments (FIG. 14B), no other obvious effect of either Natalizumab or Vedolizumab on the number of interacting cells, or speed, or dwell time or straightness was detected, comparing to untreated controls. These results show that neither Natalizumab nor Vedolizumab may affect leukocyte recruitment on P and E selectin substrates.

In the Natalizumab and Vedolizumab efficacy tests (described in Examples 5 and 12), little or no cell interaction will be detected if the drug efficacy is close or above maximal. To exclude the possibility that blood cells may be damaged during the blood collection process, the P and E selectin assay provides a suitable internal control assay to ensure the viability of the blood cells. Similarly, P and E selectin assays may also be used as an internal control for the detection of efficacy for other anti-adhesion drugs, including but not limited to Etrolizumab, Efalizumab, PTG-100 and Bio-1211.

### Example 17: Assessment of the Functions of Leukocyte Expressing CXCR1 and CXCR4

The present example is directed to use leukocyte adhesive function assay exemplary embodiments to assess the functions of leukocyte expressing CXCR1 and CXCR4, and their effects on leukocyte recruitment on VCAM-1 substrate. The protocol set forth in Appendix I was used with the following modifications to perform the experiments:

1. Microfluidic channels were pre-coated with one of the following substrate and/or substrates before being used for the assays:
VCAM-1 (10 μg/ml),
VCAM-1 (10 μg/ml)+IL-8 (1 μg/ml, R&D System. Catalogue number: 208-IL),
VCAM-1 (10 μg/ml)+SDF1α (1 μg/ml, R&D System, Catalogue number: 350-NS)

IL-8 and SDF-1α are two chemokines that may guide the migration of leukocytes by forming a concentration gradient. IL-8 is shown to mainly induce neutrophil chemotaxis, whereas SDF-1α predominantly regulates T lymphocyte chemotaxis. CXCR1 and CXCR4, receptors for IL-8 and SDF1α respectively, may be expressed on leukocyte membrane, and play a role in the regulation of leukocyte migration. As a result, CXCR1 and CXCR4 μlay a role in the regulation of leukocyte migratory behaviours. In this example, IL-8 and SDF1α was used as adhesive substrate in combination with VCAM-1, so that the function of leukocyte expressing CXCR1 and CXCR4 may be assessed.

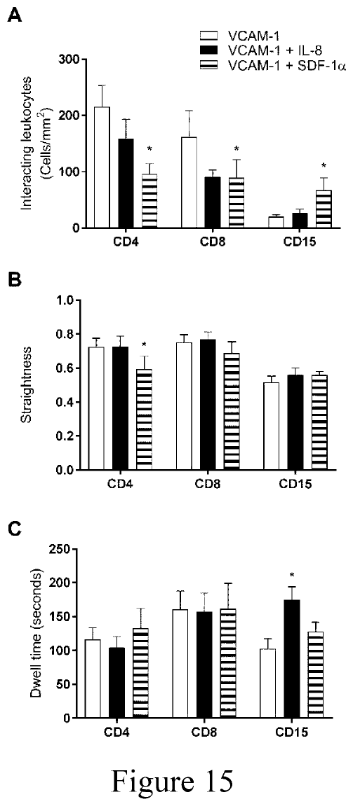

Comparing to VCAM-1 alone, SDF1α plus VCAM-1 significantly (p<0.05) reduced the number of interacting CD4 and CD8 cells, whereas the number of CD15 cells was significantly increased (FIG. 15A), showing an expression and function of CXCR4 on these white blood cells. Consistently, SDF1α also significantly (p<0.05) reduced the straightness of interacting CD4 cells, showing that CD4 cells are able to receive signals from SDF1α, confirming the functional role of CXCR4 (FIG. 15B). Additionally, the dwell time of interacting CD15 cells was significantly increased in the presence of VCAM-1 and IL-8, comparing to VCAM-1 alone, showing a functional role of CXCR1 in the regulation of CD15 cell migratory behaviours (FIG. 15C).

Together, these results show the ability of LAFA exemplary embodiments to detect the functions of chemokine receptors, and their effects on leukocyte migratory behaviours in specific leukocyte subsets. A similar strategy may then be used to assess the functions of other leukocyte expressing chemokine receptors and/or chemokines. It has been shown that the expression and function of certain chemokine receptors and/or chemokines may be activated in disease conditions. Thus, LAFA exemplary embodiments provides a suitable tool to quantitatively identify these abnormal activations, which may then be used to develop optimal treatments on personal basis and/or for one or more subjects.

### Example 18: To Predict the Likelihood of IBD Patients to Respond to Vedolizumab Therapy

The present example is directed to use leukocyte adhesive function assay exemplary embodiments to predict the patient likelihood to respond to Vedolizumab therapy. Based on the protocol described in Example 10, the following modifications were made to perform the experiments:

1. Blood was taken from patients with active inflammatory bowel diseases, who are currently not undergoing Vedolizumab therapy. To test the ability of patient leukocytes to respond to Vedolizumab, blood treated with various doses Vedolizumab (e.g. 0.01, 0.03, 0.1, 0.3, 1, 3 and 10 μg/ml), before being used for LAFA analysis.

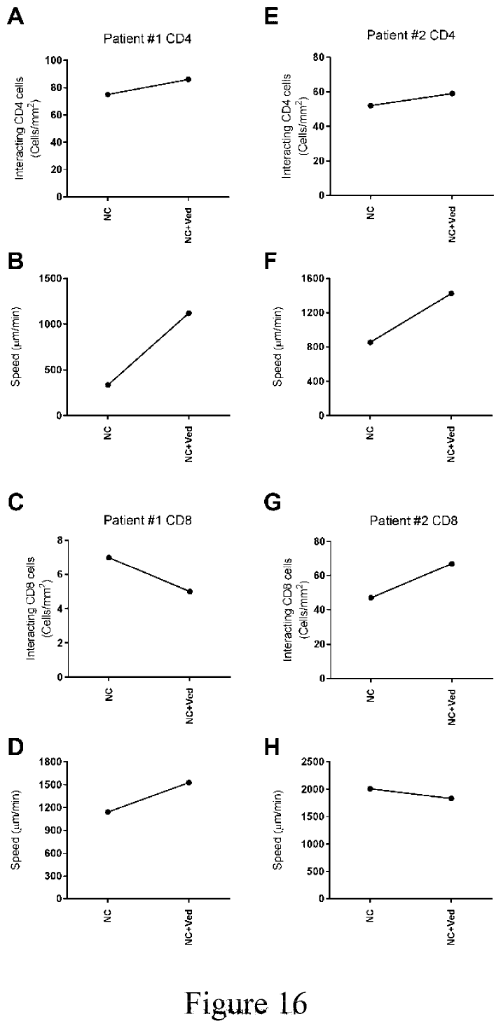

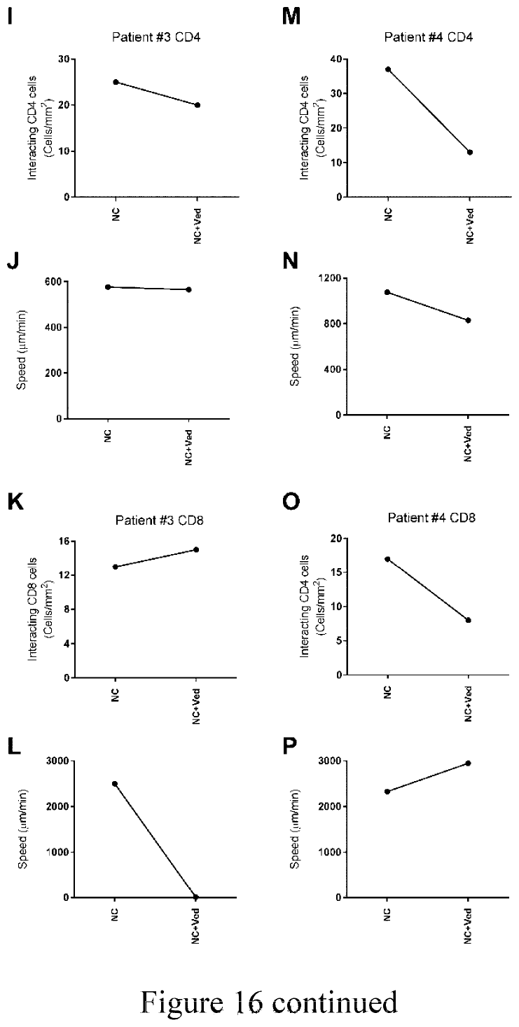

Comparing to untreated controls, Vedolizumab treatments (0.1 μg/ml) increased the speed of interacting CD4 and CD8 leukocytes in IBD patient #1 (FIGS. 16A and 16D), even though no obvious Vedolizumab effect on the number of interacting CD4 and CD8 cells was detected. These results indicated that Vedolizumab weakened the interaction between leukocytes and MAdcAM-1 substrate, leading to an increase of cell speed. These findings also demonstrated the ability of CD4 and CD8 leukocytes from patient #1 to respond to Vedolizumab treatments in vitro, based on which it may be projected that patient #1 may be likely to respond to Vedolizumab therapy. This projection is consistent with conclusion withdrawn in Examples 24 and 25, in which IBD patient #1 was also projected to respond the best to Vedolizumab treatments.

In IBD patient #2, the speed of CD4 leukocytes was also increased by Vedolizumab treatments, comparing to untreated controls, whereas no such effect was detected in CD8 leukocytes (FIGS. 16F and 16H). These results showed an ability of CD4 leukocytes from patient #2 to respond to Vedolizumab treatment in vitro, suggesting that IBD patient #2 may also respond to Vedolizumab therapy.

On the other hand, in IBD patient #3, no obvious effect of Vedolizumab on the number of CD4 and CD8 leukocytes was detected (FIGS. 161 and 16K), comparing to untreated cells. Vedolizumab treatments did not change the average speed of CD4 cells, while the speed of CD8 cell was sharply reduced (FIGS. 16J and 16L). These results showed the lack of ability of leukocytes from patient #3 to respond to Vedolizumab treatments in vitro. Accordingly, it may be predicted that patient #3 may be unlikely to respond to Vedolizumab therapy.

For IBD patient #4, comparing to untreated controls, Vedolizumab treatments caused a moderate reduction in the number of CD4 and CD8 leukocytes, showing an inhibitory effect of Vedolizumab in the recruitment of CD4 and CD8 cells on MAdCAM-1 substrate. Based on these results, it may be projected that patient #4 would also respond to Vedolizumab therapy.

Together, the results in this example show an alibility of LAFA exemplary embodiments to stratify IBD patients that are likely to respond to Vedolizumab therapy, which may potential lead to an increased clinical remission rate for Vedolizumab therapy in the stratified patient population. Additionally, similar strategies may be used to predict how likely patients would respond to other anti-adhesion therapies, including, for example, one or more of the following: PTG-100, Natalizumab, Bio-1211, Etrolizumab and Efalizumab. Other suitable anti-adhesion therapies may also be evaluated using the procedures outline in this example and/or other exemplary embodiments.

### Example 19: Detection of Drug Efficacy in MS Patients Undergoing Natalizumab Efficacy

The present example is directed to use leukocyte adhesive function assay exemplary embodiments to assess Natalizumab efficacy in multiple sclerosis (MS) patients undergoing Natalizumab therapy, according to certain exemplary embodiments. Based on the protocol described in Example 1, the following modifications were made to that protocol to perform the experiments:

1. Blood was Taken from MS Patients Who are not Currently Undergoing Vedolizumab Therapy.

Blood samples were collected at various time points (2, 4, 6, and 10 weeks) post drug infusion, and then were analysed by LAFA exemplary embodiments using VCAM-1 as adhesive substrate. MS patients on Copaxone therapy was used as negative control group, as Copaxone is unlikely to have any impacts on α4 integrin functions.

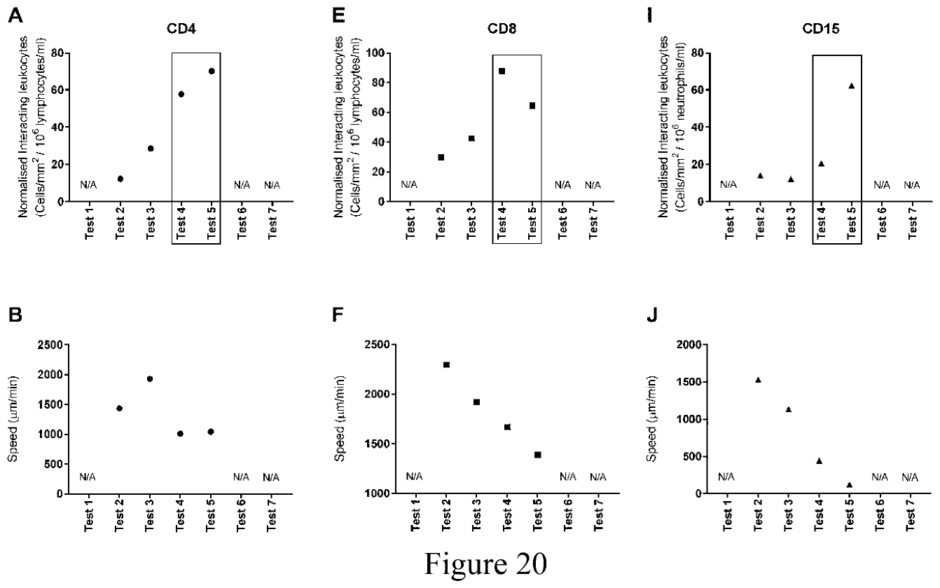

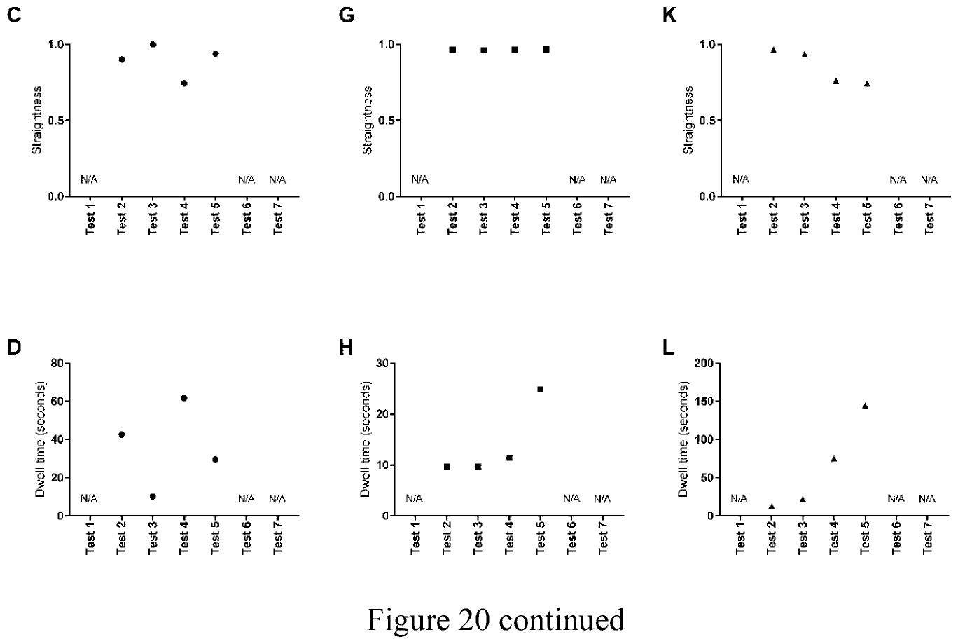

In the absence of Mn²⁺, as shown in FIG. 20A, a large number of interacting CD4 cells (326.9 cells/mm²) were detected in the Copaxone patients. On the other hand, only background level of CD4 cells (11.5 cells/mm²) was observed at week 2 after Natalizumab infusion, showing that CD4 cell interaction with VCAM-1 substrate was completely, or substantially, inhibited in this patient. Similarly, no obvious CD4 recruitment on VCAM-1 substrate was detected in blood samples collected at week 4 post drug infusion (FIG. 20A), indicating a 100% Natalizumab efficacy.

At week 6 post Natalizumab infusion, however, a small number (23.9 cells/mm²) of interacting CD4 cells was detected, and a more obvious CD4 cell recruitment (70.1 cells/mm²) was observed at week 10 post drug infusion, showing a lower Natalizumab efficacy than maximum at these time points (FIG. 20A). It was also noted that the dwell time of interacting CD4 cells was increased by Mn²⁺ treatments at week 6 and 10 (not at week 2 and 4) post Natalizumab infusion, confirming the drug efficacy was reduced below maximum at week 6 and 10 (FIG. 20B).

Together, these results show an excellent ability and/or sensitivity of LAFA exemplary embodiments to accurately and quantitatively assess drug efficacy in patients undergoing Natalizumab therapy. As Natalizumab sensitivity may vary dramatically with time and between individual subjects, LAFA exemplary embodiments provide a fast and accurate tool to monitor drug efficacy on personal basis, facilitating the development of personalised treatment regimens to maximise drug therapeutic benefits and minimise drug-induced side effects. Additionally, similar strategy may be used to monitor efficacy of other anti-adhesion drugs, including one or more of the following: Vedolizumab, Efalizumab, PTG-100 and Bio-1211. Other suitable anti-adhesion therapies may also be evaluated using the procedures outline in this example and/or other exemplary embodiments.

### Example 20: The Uses of LAFA as Point-of-Care Tests in Clinical Settings

The present example is directed to use leukocyte adhesive function assay exemplary embodiments to develop a range of point-of-care tests.

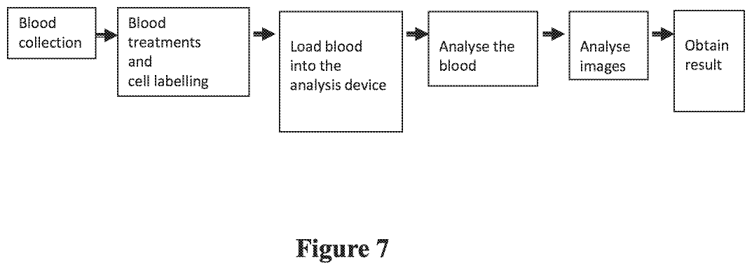

For example, the techniques described herein may be used to develop a point-of-care blood test which detects the effectiveness of Natalizumab in vitro. The blood test may comprise an analysis device, a microfluidic system (e.g. a microfluidic pump and a microfluidic chip), analysis algorithm/s, data transmission platform and related reagents. As seen in FIG. 7, users/patients may: 1. obtain a blood sample from a fingertip by a finger prick; 2. load the blood into a chip; 3. insert the chip into the analysis device; and 4. obtain a result.

The point of care blood test may allow direct assessment of Natalizumab functions, regardless of serum drug concentrations. No current technology has such capability. The blood test will only require <100l (finger prick amount, e.g. 10, 20, 30, 40, 50, 60, 70, 80, 90, 100, 120, 140, 160, 180, 200, 250, 300, 350, 400, 500, 600, 700, 800, 900 and 1,000 μl) of whole blood and may provide results in under 30 minutes (e.g. 5, 10, 15, 20, 25, 30, 40, 50, 60, 90, 120, 180, 240 minutes). The blood test may be implemented into current treatment regimens as it may be utilised at various time points post Natalizumab administration, informing the need for re-dose Natalizumab infusions only when a reduction of the drug effectiveness is detected by the blood test. As a result, the blood test may facilitate the development of a personalised treatment regimen for individual patients. It is aimed to adapt the blood test to be compatible with existing devices and/or platforms, or even a smart phone to enable remote data sharing with GPs/specialists, leading to a significantly reduced workload for busy MS specialists.

In addition, other clinical applications may also be developed from LAFA exemplary embodiments, including but not limited to:
1. Point of-care test to determine the efficacy of other drugs, including but not limited to, Vedolizumab, Etrolizumab, Efalizumab, PTG-100 and Bio-1211.
2. Optimising treatment regimens on personal basis to reduce the risk of drug-induced side effects.
3. Monitoring healthy status of a subject or subjects.
4. Identifying markers for leukocyte adhesive functions in individual patients.
5. Identifying altered leukocyte adhesive functions (related to healthy controls).
6. Projecting the likelihood of a subject or subject to respond to certain therapies.
7. Recommending suitable treatments for a subject or subjects with unknown aetiology or without disease diagnosis.

### Example 21: To Create Personal Profile for Leukocyte Adhesive Functions (Leukocyte Adhesion Fingerprint)

The present example is directed to use leukocyte adhesive function assay exemplary embodiments, according to certain exemplary embodiments, to analyse multiple blood samples collected from a single healthy subject at various time-points. The LAFA exemplary embodiments used in this example is detailed in Examples 1, 10 and 16. Blood samples were analysed by LAFA exemplary embodiments, using VCAM-1 (α4β1 integrin ligand), MAdCAM-1 (α4β7 integrin ligand) and P-selectin (PSGL-1 ligand) as substrates. As a result, a personal profile for basal level of leukocyte adhesive functions was created, as detailed in Examples 3 and 11. Blood was also treated with 5 mM MnCl₂ before being used for LAFA analysis, so that the Mn²⁺ induced activation potential can be determined, as detailed in Example 4.

Seven blood tests (from Test1 to Test7) were performed using LAFA exemplary embodiments. The blood samples were collected from a healthy individual at various time-points during a period of approximately three months, in week 1, week 9, week 11, week 12, week 13, week 15 and week 16. The original goal of this example was to use the data from these 7 blood tests to create a personal leukocyte adhesive function profile for this healthy individual. However, this individual was suffered from wisdom tooth pain (inflamed gum near the wisdom tooth) on the day when blood test #5 (week 13) was performed. As presented in detail below, abnormal leukocyte activation was detected not only in blood test #5, but also in blood test #4 (week 12), which was performed 7 days before blood test #5.

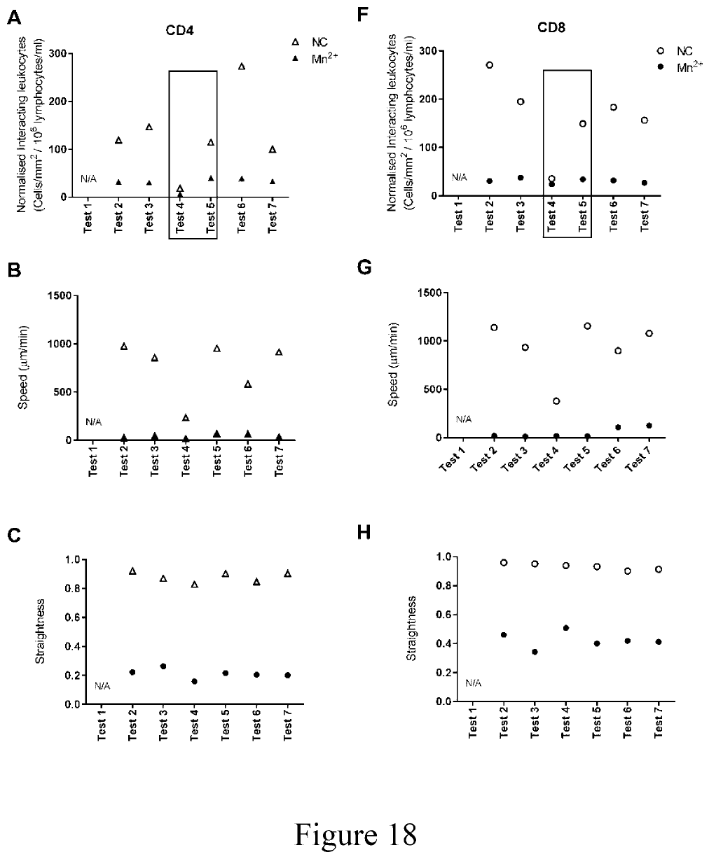

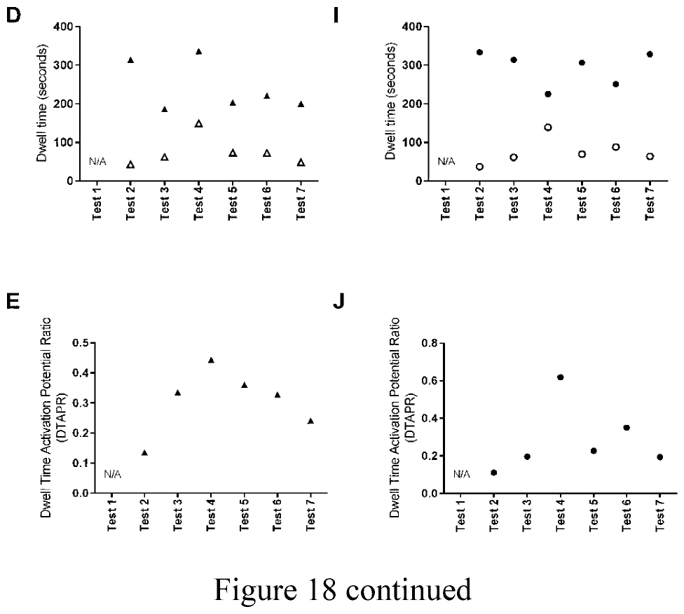

On VCAM-1 substrate, very little variation of CD4 and CD8 cell straightness was observed amount in Tests #1-6. This demonstrates the ability of LAFA exemplary embodiments to produce consistent results at different time points (FIGS. 18C and 18H). However, an abnormally low leukocyte speed in resting CD4 and CD8 leukocytes was observed in blood test #4 comparing to the results from other blood tests, indicating an activation of these leukocytes. Furthermore, a high dwell time in resting CD4 and CD8 cells was also detected (FIGS. 18D and 18I). Also the Dwell Time Activation Potential Ratio (DTAPR, described in Example 4) of CD4 and CD8 cells was high in blood test #4, showing a high portion of activated α4β1 integrin, compared to other blood samples tested in the series. These findings show an abnormal activation of α4β1 integrin on CD4 and CD8 leukocytes in blood test #4, which was performed 7 days before the gum inflammation. These results demonstrate the use DTAPR as a marker to detect early signs of immune response in systemic and/or local inflammation.

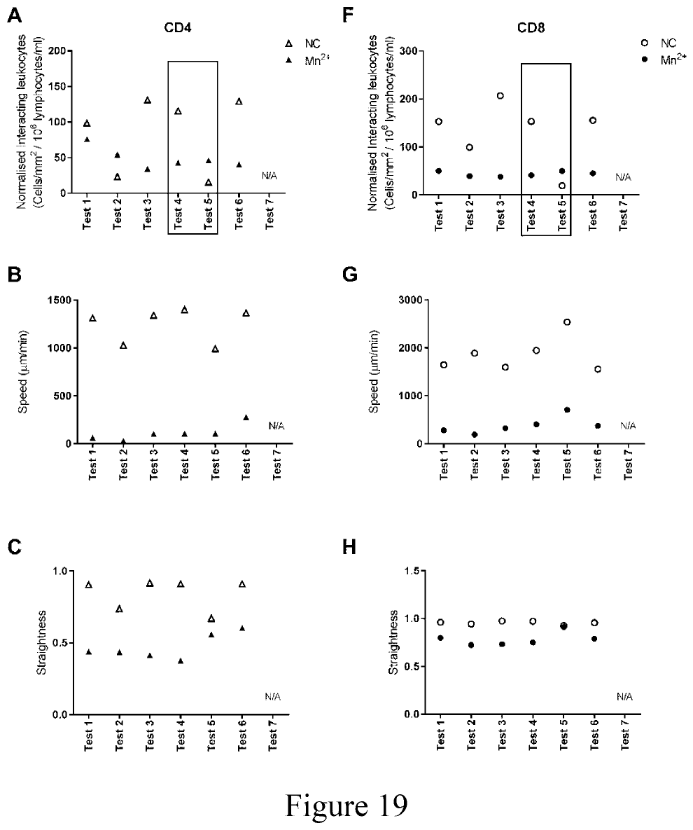

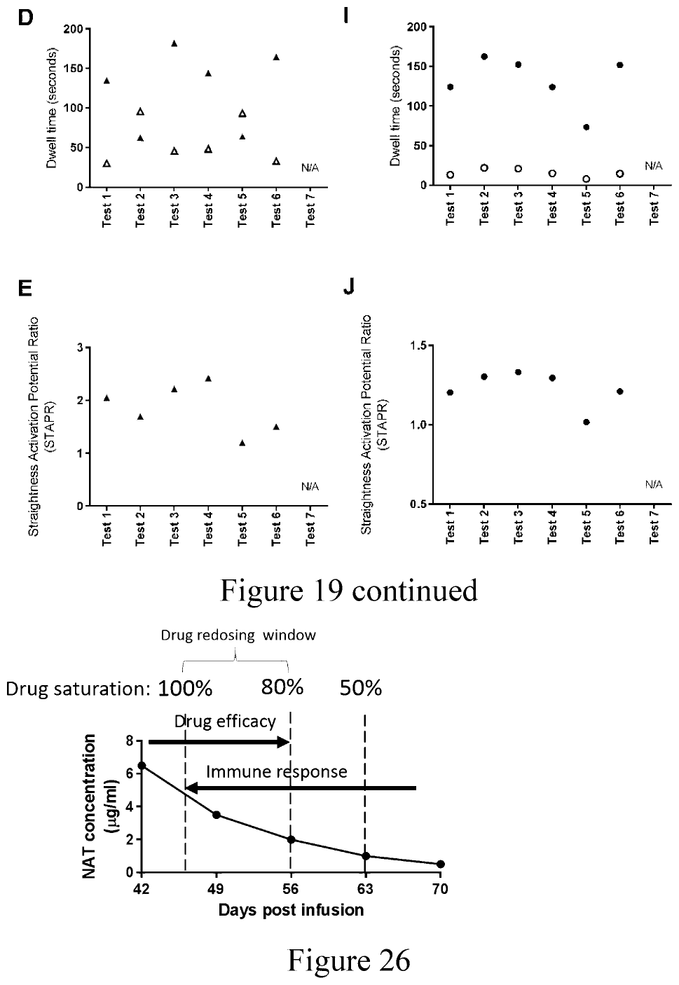

On MAdCAM-1 substrate, little variation in the average cell speed was detected in CD4 and CD8 leukocytes, showing a good consistency of LAFA exemplary embodiments to assess leukocyte adhesive functions. However, a low straightness was observed in resting CD4 cells in blood test #5, when the wisdom tooth pain occurred (FIG. 19C). The dwell time of resting CD4 cells was also abnormally high in test #5, compared to the values found from other blood samples tested in the series (FIG. 19D). The value of Straightness Activation Potential Ratio (STAPR) was low in both CD4 and CD8 leukocytes, showing a high portion of activated α4β7 integrin on CD4 and CD8 leukocytes in blood test #5 (FIGS. 19E and 19J). These results demonstrate the use of ATAPR as marker for the assessment and/or prediction of immune response in systemic and/or local inflammation.

On P-selectin substrate, a high number of interacting CD4 and CD8 cells were detected in blood test #4 and #5 and when compared to other tests results, shows an unusual PSGL-1 (P-selectin ligand) activation in these leukocytes (FIGS. 20A and 20E). Additionally, the number of CD15 cells in blood test #5 is almost 8-fold higher than the average of other tests, indicating a highly activated PSGL-1 on CD15 leukocyte (FIG. 20I). The average cell speed of CD4, CD8 and CD15 cells were abnormally low in blood tests #4 and #5, suggesting a strong interaction between these leukocytes and P-selectin substrate (FIGS. 20C, 20G and 20K). The dwell time of CD8 and CD15 cells was abnormally high in blood test #5, when compared to other blood test results. This shows that PSGL-1 on CD8 and CD15 leukocytes is highly activated, leading to an enhanced interaction between leukocytes and P-selectin substrate.

Together, these findings demonstrate the ability of LAFA exemplary embodiments to create personal profile for basal level of inflammatory status in a subject. Despite a wisdom tooth pain, several parameters of leukocyte migratory profile were not affected by this local gum inflammation, including cell straightness on VCAM-1 substrate (FIGS. 18C and 18 H), and cell speed in MAdCAM-1 substrate (FIGS. 19B and 19G). These findings demonstrate a good reproducibility of LAFA exemplary embodiments for detection of leukocyte adhesive functions.

Based on this basal level of inflammatory status, however, the effects of the gum inflammation on a range of other parameters were clearly detectable up to 7 days before the acute inflammation. Our results clearly showed the activation of α4β1 integrin, α4β7 integrin and PSGL-1 in blood tests #4 and #5. It was also noted that the effects of the local gum inflammation differ dramatically between different parameters in different leukocyte subsets. Thus, these results show the potential uses of the changes in specific parameters on specific leukocyte subsets to determine early sign of abnormal immune responses, which may then be used to predict the likelihood of certain diseases.

Creation of “Leukocyte Adhesion Fingerprint”

With the ability to assess adhesive functions of multiple leukocyte adhesion molecules in specific leukocyte subsets, it becomes possible to quantitatively characterise leukocyte adhesive functions in much greater detail than ever before. The adhesive functions of multiple leukocyte adhesion molecules may be studied in individual flow channels, which are pre-coated with specific adhesive endothelial molecule substrates. As a result cell migration profiles for each adhesion molecule on a specific leukocyte subset may be generated. Combined with advanced bioinformatics, leukocyte adhesion fingerprints may be generated for individual subjects/patients. The leukocyte adhesion fingerprint may serve as a useful tool for identifying leukocyte abnormalities. Thus, the potential applications for leukocyte adhesion fingerprints include:
Determination of personalised pathogenesis
Identification of new disease markers for diseases
Identification of early signs of diseases
Assist early and accurate diagnosis
Disease prediction
Monitoring health status in healthy people (routine health check)

Some embodiments are able to simultaneously assess adhesive functions of multiple leukocyte adhesion molecules on multiple leukocyte subsets using unprocessed whole blood. Potential applications of certain exemplary embodiments may not be limited to certain leukocyte molecules, or certain leukocyte subsets, or certain species of mammals.

### Example 22: To Assess Basal Inflammatory Status of Leukocyte Molecules in Patients with Multiple Sclerosis (MS) and Inflammatory Bowel Diseases (IBD)

The present example is directed to identify abnormal activation of leukocyte adhesive functions in patients with multiple sclerosis (MS) and/or inflammatory bowel diseases (IBD) patients, comparing with healthy subject controls, according to certain exemplary embodiments. Blood samples were taken from MS and/or IBD patients, and then used for leukocyte adhesion assay analysis using VCAM-1 and MAdCAM-1 as substrates, as detailed in Examples 1 and 10. The basal inflammatory status of α4β1 integrin and α4β7 integrin were calculated as detailed in Examples 3 and 11.

To compare basal inflammatory status of healthy subject controls with patients with MS and/or IBD, Relative Straightness Index (RSTI), Relative Speed Index (RSI) and Relative Dwell Time Index (RDTI) were employed, as detailed in Example 3 and 11. The criteria used in this example for selecting a health subject for use in the control group is set forth in example 3.

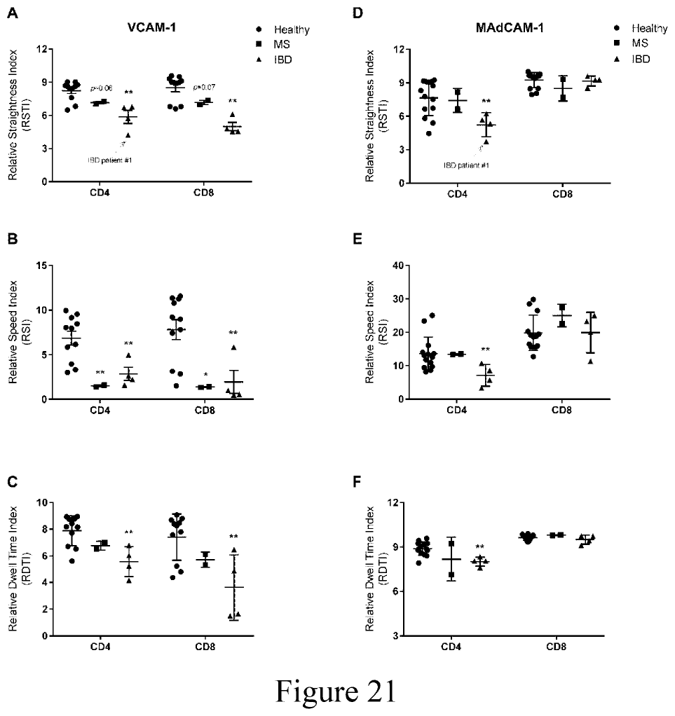

As shown in FIG. 21A, significantly lower RSTI values in CD4 and CD8 leukocytes were observed in both MS and IBD patients comparing to healthy controls, showing an enhanced α4β1 integrin activation in MS and IBD cells. Significantly lower RSI values in CD4 and CD8 leukocytes were also detected in MS and IBD patients, indicating an abnormally increased basal inflammatory status of α4β1 integrin in MS and IBD cells (FIG. 21B). Consistently, a reduced RDTI value was also detected in MS and IBD CD4 and CD8 leukocytes (FIG. 21C). These results clearly show abnormal activation of α4β1 integrin in MS and IBD patients.

A shown in FIG. 21D, significantly lower RSTI values were detected in CD4 leukocytes from IBD patients compared to healthy controls, showing a highly activated α4β7 integrin in CD4 leukocytes in IBD patients. Consistently, a lower RSI and RDTI value was also observed in CD4 leukocyte from IBD patients. On the other, no such activation of α4β7 integrin were observed in MS patients, showing a normal α4β7 integrin function in MS CD4 leukocytes, related to healthy controls.

Together, these results clearly show the ability of LAFA exemplary embodiments to detect abnormal activation of α4β1 integrin and α4β7 integrin on specific leukocyte subsets in MS and IBD patients. As shown in FIGS. 18C and 18H in Example 23, leukocyte straightness is not typically affected even by a local inflammation, such as wisdom tooth pain. Thus, the lower RSTI values in MS and IBD leukocytes may be used as a specific disease marker for one or more of the following: I. Assist disease diagnosis, 2. Stratify patient subpopulations, 3. Detect early signs of diseases, and 4. Optimise treatment conditions on personal basis. The uses of RSTI RSI and RDTI facilitates the application of the technology in clinical settings.

### Example 23: To Assess Mn²⁺-Induced Activation Potential of Leukocyte Molecules in Patients with Multiple Sclerosis (MS) and Inflammatory Bowel Diseases (IBD)

The present example is directed to assess Mn²⁺-induced activation potential of leukocyte molecules in MS and IBD patients. Blood samples were taken from MS and IBD patients, and then used for leukocyte adhesion assay analysis using VCAM-1 and MAdCAM-1 as substrates, as detailed in Examples 1 and 10. The Mn²⁺ activation potential of α4β1 integrin and α4β7 integrin was calculated as detailed in Example 4.

To compare basal inflammatory status of healthy control with patients with MS and IBD, Straightness Activation Potential Ratio (STAPR), Speed Activation Potential Ratio (SAPR) and Dwell Time Activation Potential Ratio (DTAPR) were employed, as detailed in Example 4.

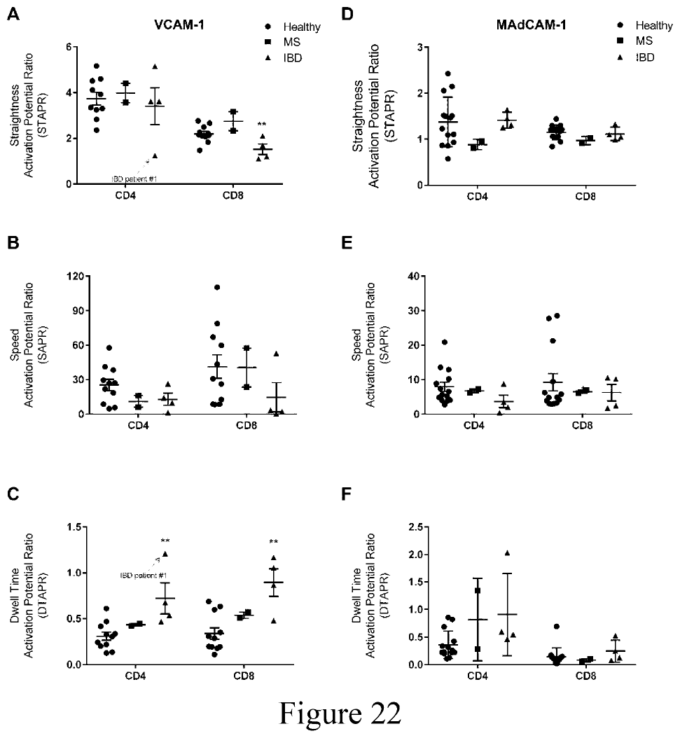

As shown in FIG. 22A, the STAPR value of IBD CD8 leukocytes on VCAM-1 substrate is significantly (p<0.01) lower than healthy controls, suggesting a high portion of activated α4β1 integrin in IBD CD8 cells and lower Mn²⁺-induced activation potential. Consistently, comparing to healthy controls, a significantly higher DTAPR values in IBD CD8 leukocytes was observed (FIG. 22C). It was noted that the DTAPR values of IBD CD4 leukocytes were also higher than healthy controls, suggesting a lower Mn²⁺-induced activation potential in IBD CD4 cells (FIG. 22C). No obvious difference in STAPR, SAPR or DTAPR in MS and IBD patient on MAdCAM-1 substrate was detected, comparing to healthy controls (FIGS. 22D to 22F).

Together, these results clearly show the ability of LAFA exemplary embodiments to detect abnormal Mn²⁺-induced activation potential of α4β1 integrin and α4β7 integrin on specific leukocyte subsets in MS and IBD patients. Thus, STAPR, SAPR or DTAPR values may be used as highly specific disease marker to: 1. Assist disease diagnosis, 2. Stratify patient subpopulations, 3. Detect early signs of diseases, and 4. Optimise treatment conditions on personal basis. The uses of STAPR, SAPR or DTAPR should facilitate the application of certain exemplary embodiments to clinical settings.

### Example 24: To Use Basal Inflammatory Status to Predict the Likelihood of a Subject or Subjects to Respond to Anti-Adhesion Therapy

The present example is directed to use basal inflammatory status of leukocyte molecules to predict the likelihood of a subject or subjects to respond to anti-adhesion therapy, including but not limited to monoclonal antibodies, small molecules, compounds and peptides. The calculation of basal inflammatory status of leukocyte adhesion molecules is detailed in Examples 3 and 11.

In exemplary embodiments, the basal inflammatory status of leukocyte molecules may be used to stratify patient subpopulations with highly enhanced activity in these molecules, including but not limited to α4β1, α4β7 integrin, α_{L} integrin. GM integrin, CXCR1 and CXCR4.

Several drugs (including but not limited to Natalizumab, Vedolizumab, Etrolizumab, PTG-100, Bio-1211 and Efalizumab) have been or are being developed to inhibit the functions of leukocyte adhesive molecules, therefore, attenuate disease progression. Patient sub-population with high basal inflammatory status may be predicted to be more likely to respond to the treatments that inhibit the functions of these molecules.

For example, as shown in FIG. 21A, IBD patient #1 had the lowest RSTI value on VCAM-1 substrate amount all 4 IBD patients, indicating patient #1 had the most activated α4β1 integrin. Based on this result, it can be predicted that IBD patient #1 may respond the best to therapies that inhibit α4β1 integrin functions (e.g. Natalizumab or Bio-1211).

Similarly, as shown in FIG. 21D, IBD patient #1 also had the lowest RSTI value on MAdCAM-1 substrate amount all 4 IBD patients, suggesting patient #1 had the most activated α4β7 integrin. Based on this result, it can be predicted that patient #1 may respond the best to therapies that inhibit α4β7 integrin functions (e.g. Vedolizumab or PTG-100).

Together, our results show the ability of RSI, RDTI and RSTI as marker to predict the likelihood of a subject or subjects to respond to specific therapies.

Accordingly, these data clearly show that LAFA may be used for patient grouping/stratification. For example, despite the fact that α4β1 integrin is activated in MS patients, the basal inflammatory status of α4β1 integrin may vary dramatically between individual patients. The semi-quantitative tools generated herein allow the determination of the level of activation of α4β1 integrin in individual patients. As a result, patients with highly activated α4β1 integrin may be identified and separated from patients with low or no α4β7 integrin activation. It can then be projected that Natalizumab therapy will work more effectively in MS patients with highly activated α4β1 integrin. compared to other patient groups.

### Example 25: To Use Mn²⁺-Induced Activation Potential to Predict the Likelihood of a Subject or Subjects to Respond to Anti-Adhesion Therapy

The present example is directed to use Mn²⁺-induced activation potential to predict the likelihood of a subject or subjects to respond to anti-adhesion therapy, including but not limited to monoclonal antibodies, small molecules, compounds and peptides. The calculation of Mn²⁺-induced activation potential (e.g. Straightness Activation Potential Ratio (STAPR), Speed Activation Potential Ratio (SAPR) and Dwell Time Activation Potential Ratio (DTAPR)) of leukocyte adhesion molecules is detailed in Example 4.

In exemplary embodiments, the Mn²⁺ induced activation potential of specific leukocyte molecules may be also used to stratify patient subpopulations with enhanced portion of activated form of these molecules (or low Mn²⁺ induced activation potential), including but not limited to α4β1 integrin, α4β7 integrin, α3β1 integrin, αVβ3 integrin, αLβ2 integrin, αIIbβ3 integrin, α6β1 integrin, α1β1 integrin, α2β1 integrin, αvβ3 integrin and α5β1 integrin.

Several drugs (including but not limited to Natalizumab, Vedolizumab, Etrolizumab. PTG-100, Bio-1211 and Efalizumab) have been or are being developed to inhibit the functions of leukocyte adhesive molecules, therefore, attenuate disease progression. Patient sub-population with high portion of activated forms of these molecules (or low Mn²⁺ induced activation potential) may be predicted to be more likely to respond to the treatments that inhibit the functions of these molecules.

For example, despite no significant difference in STAPR between healthy control and IBD patients, IBD patient #1 had the lowest STAPR value amount all IBD patients, suggesting a highest portion of activated α4β1 integrin in this patient. Based on this result, it can be predicted that IBD patient #1 may respond the best to therapies that inhibit α4β1 integrin functions (e.g. Natalizumab or Bio-1211). This prediction is identical to the prediction based on the RSTI values in Example 24. where the same patient (IBD #1) was predicted to respond the best to anti-α4β1 integrin therapies.

Together, our results show the ability of STAPR, SAPR and DTAPR as markers to predict the likelihood of a subject or subjects to respond to specific therapies.

### Example 26: Recommendation of Specific Anti-Adhesion Therapy Based on Results from Leukocyte Adhesive Function Assay (LAFA)

The present example is directed to use leukocyte adhesive function assay exemplary embodiments to provide recommendations of specific therapies to patients with unknown aetiology and/or prior to disease diagnosis, after the disease diagnosis or combinations thereof.

LAFA exemplary embodiments may be used to directly assess the ability of leukocytes from a subject or subject to respond to certain drugs, including but not limited to monoclonal antibodies, small molecules, compounds and peptides. Based on these results, the likelihood of a subject or subjects to specific therapies may then predicted, as detailed in Example 18.

LAFA exemplary embodiments allow a quantitative assessment of the functions of a range of leukocyte expressing molecules, including but not limited to α4β1 integrin, α4β7 integrin, PSGL-1. CXCR1 and CXCR4, leading to a semi-quantitative assessment the basal inflammatory status of these molecules, as detailed in Examples 3 and 11. Based on the basal inflammatory status of these molecules, the likelihood of a subject or subjects to specific therapies may then predicted, as detailed in Example 24.

Similarly, LAFA exemplary embodiments also offer a semi-quantitative tool to assess Mn²⁺-induced activation potential so that the portion of activated leukocyte molecules on specific leukocyte subsets may be determined. Based these results, the likelihood of a subject or subjects to specific therapies may then predicted, as detailed in Example 25.

LAFA exemplary embodiments may be used to develop treatments for a subject (or subjects) with unknown aetiology and/or prior to disease diagnosis. For example, comparing to healthy controls, if altered α4β1 integrin activation is identified in a subject (or subjects), it may then be predicted that anti α4 integrin therapy (e.g. Natalizumab) may be suitable for the treatments for this subject and/or these subjects, prior to disease diagnosis in the subjects and/or subjects.

In exemplary embodiments, comparing to healthy controls, the detection of altered α4β1 integrin activity in one or more subjects may indicate that an anti α4 integrin therapy (e.g. Natalizumab) should be given to the one or more subjects for the treatment of these one or more subjects. In exemplary embodiments, the detection of altered α4β1 integrin activity in one or more subjects indicates that an anti α4 integrin therapy (e.g. Natalizumab) should be given to the one or more subjects in order to attenuate disease progression in the one or more subjects, wherein the anti α4 integrin therapy is given to the one or more subjects, prior to disease diagnosis in one or more subjects. In certain exemplary embodiments, the detection of the basal inflammatory status of α4β1 integrin activation state may be used to indicate an anti α4 integrin treatment may be suitable for one or more subjects with unknown aetiology and/or prior to disease diagnosis, after the disease diagnosis or combinations thereof. In certain exemplary embodiments, the detection of the Mn²⁺-induced activation potential of α4β1 integrin may be used to indicate an anti α4 integrin treatment may be suitable for one or more subjects with unknown aetiology and/or prior to disease diagnosis, after the disease diagnosis or combinations thereof.

As shown in Table 3, a range of anti-adhesion drugs have been developed to inhibit the functions of these leukocyte molecules, aiming to attenuate disease progression. Thus, a similar strategy may be used to identify altered activation of other leukocyte molecules (e.g. pi integrin, 37 integrin, PSGL-1, CXCR1 and/or CXCR4) in a subject and/or subjects, based on which suitable treatment strategies may be developed with unknown aetiology and/or prior to disease diagnosis, after the disease diagnosis or combinations thereof.

In addition, LAFA exemplary embodiments may be used to identify new applications for certain drugs, including but not limited to Natalizumab, Vedolizumab, Efalizumab, Etrolizumab, PTG-100 and Bio-1211. For example, the application of Natalizumab therapy is currently limited to MS and Crohn's disease. Using the quantitative approaches developed herein, blood samples from patients with other inflammatory diseases may be analysed, leading to identification of patient populations with increased α4 integrin functions, as detailed in Examples 18, 24 and 25. Thus, an ability of Natalizumab therapy to control disease progression in these patient populations is expected. As a result, the application of Natalizumab therapy may be extended to other human diseases.

### Example 27: To Determine Drug Sensitivity of Anti-Adhesion Therapies in Individual MS and IBD Patients

The present example is directed to use leukocyte adhesive functions assay exemplary embodiments to determine levels of drug sensitivity in individual patients using IC50 values, as detailed in Examples 5 and 12.

For example, to quantitatively assess Natalizumab sensitivity in multiple sclerosis (MS) patients, blood samples were collected from MS patients, and then used for LAFA analysis using VCAM-1 as substrate, as detailed in Example 5. As shown in Table 5, the Natalizumab IC50 value of patient #1 (0.2657 μg/ml) is almost 4 fold higher than patient #2 (0.06843 μg/ml), suggesting that patient #1 is 4 time more resistant to Natalizumab than patient #2. In addition, the Natalizumab IC50 value of patient #1 is also higher than the average IC50 values in healthy control (0.03 μg/ml), as shown in Table 1.

TABLE 5
Natalizumab IC50 values of individual MS patients. Blood samples were
collected from MS patients, and then used for LAFA using VCAM-1 as
substrate, as detailed in Example5. The IC50 value for each patient was
then determined as detailed in Example 5.

Patient

| | MS Patient #1 | MS Patient #2 |
|---|---|---|
| Natalizumab IC50(μg/ml) | 0.2657 | 0.06834 |

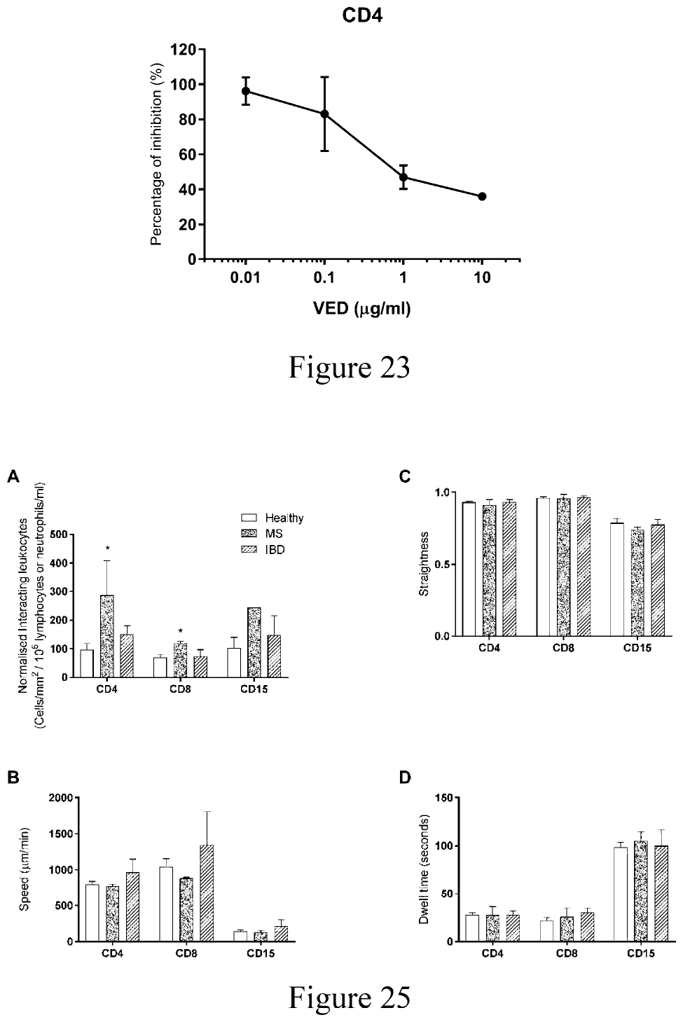

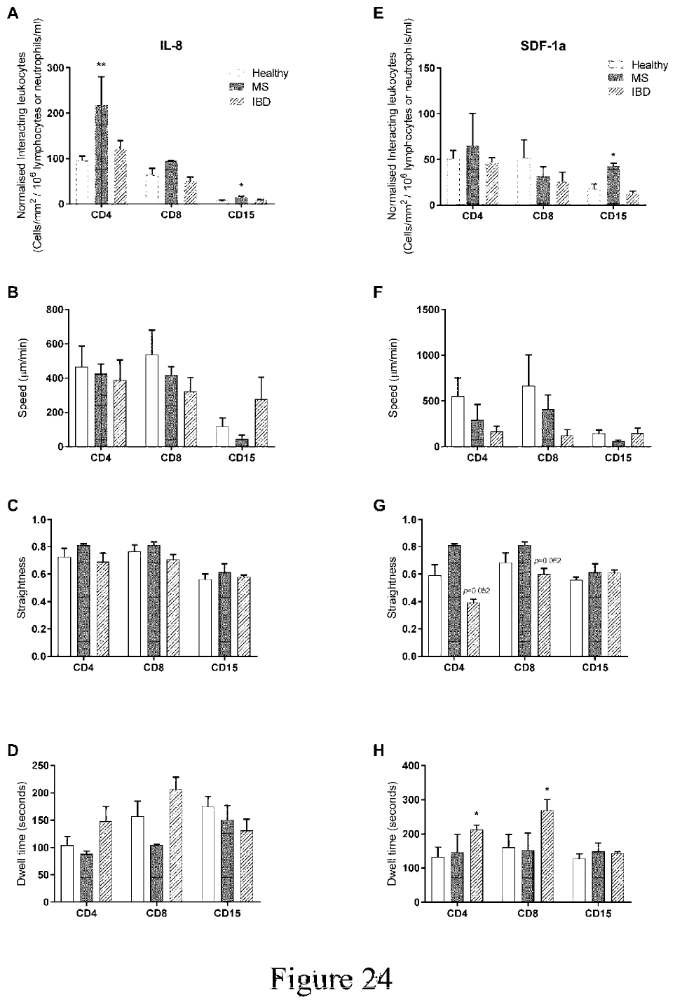

In another example, to quantitatively assess Vedolizumab sensitivity in patients with, blood samples were collected from IBD patients, and then used for LAFA using MAdCAM-1 as substrate, as detailed in Example 12. It was shown in FIG. 10A and Example 12 that Vedolizumab did not inhibit the number of resting CD4 leukocytes on MAdCAM-1 substrate in healthy subjects. As show in FIG. 23, however, the number of interacting CD4 leukocytes was gradually reduced with an increase of Vedolizumab concentrations, suggesting the ability of Vedolizumab to inhibit the recruitment of IBD CD4 leukocytes. The Vedolizumab IC50 values were also determined in three individual IBD patients. As show in Table 6, the Vedolizumab IC50 value was over 2.5 fold higher in patient #3 (2.573 μg/ml) than patient #2 (1.009 μg/ml), suggesting a large variability in Vedolizumab sensitivities amount different IBD patients.

TABLE 6
Vedolizumab IC50 values of individual IBD patients. Blood samples were
collected from IBD patients, and then used for LAFA using MAdCAM-1
as substrate, as detailed in Example 12. The IC50 value for each patient
was then determined as detailed in Example 12.

| | Patient | | |
|---|---|---|---|
| | IBD patient #1 | IBD patient #2 | IBD patient #3 |
| Vedolizumab IC50 | 1.628 | 1.009 | 2.573 |
| (μg/ml) | | | |

Together, these results show the ability of LAFA exemplary embodiments to use IC50 values to quantitatively assess different levels of drug sensitivity in individual patients. The results from LAFA exemplary embodiments may facilitate the development of optimal dosage regimens on personal basis for a wide range of drug and/or treatments.

### Example 28: To Assess the Functions of Leukocyte Expressing CXCR1 and CXCR4 in Patients with Multiple Sclerosis (MS) and Inflammatory Bowel Diseases (IBD)

The present example is directed to assess the functions of leukocyte expressing CXCR1 (IL-8 receptor) and CXCR4 (SDF1α receptor) in MS and IBD patients. Blood samples were collected from patients, and then analysed by LAFA exemplary embodiments using VCAM-1+IL-8 or VCAM-1+SDF1α as substrates, as detailed in Example 17.

As shown in FIG. 25, compared with healthy controls, the number of interacting CD4 (p<0.01) and CD15 (p<0.05) leukocytes was significantly increased in MS patients on VCAM-1+IL-8 substrates, suggesting an activated CXCR1 functions in MS patients (FIG. 25A). The number of interacting MS CD15 cells on VCAM-1+SDF1α substrates was increased, suggesting an increased activity of CXCR4 on MS CD15 leukocytes (FIG. 25E).

In addition, comparing healthy controls, the dwell time of CD4 and CD8 leukocytes on VCAM-1+SDF1α was significantly (p<0.05) increased in IBD patients, showing an enhanced activation of CXCR4 in IBD CD4 and CD8 leukocytes. Consistently, the straightness of IBD CD4 and CD8 leukocyte was reduced (almost reached statistically significance) comparing to healthy, showing an enhanced ability of IBD CD4 and CD8 cells to receive signals from SDF1α via CXCR4.

Together, these results clearly show the ability of LAFA exemplary embodiments to detect abnormal activation of leukocyte expressing CXCR1 and CXCR4 in patients with MS and IBD. Thus, these tests may also be used to assess the functions of other leukocyte chemokine receptors in other diseases. Based on the results from the assays, patients may then be stratified and/or more targeted therapies may then be developed on personal basis. For example, if a inhibitory effect of a drug and/or a compound and/or antibody and/or peptide on CXCR1 function is identified by LAFA in the drug database (as detailed in Example 15), this drug may be projected to have therapeutic benefit for a subject or subjects with enhanced activation of CXCR1.

### Example 29: To Assess the Adhesive Functions of Leukocyte Expression PSGL-1 in MS and IBD Patients

The present example is directed to use leukocyte adhesive functions assay exemplary embodiments to assess the adhesive function of leukocyte P-selectin glycoprotein ligand 1 (PSGL-1, ligand for P and E selectin) in patients with multiple sclerosis (MS) and inflammatory bowel diseases (IBD), as detailed in Example 16.

As shown in FIG. 25A, comparing to healthy controls, an increased number of CD4, CD8 and CD15 leukocytes was detected in MS patients, suggesting an elevated PSGL-1 function in these MS leukocytes. On the other hand, no changes in cell speed, straightness or dwell time was detected in MS and IBD leukocytes was observed, comparing to healthy controls (FIGS. 25B to 25D).

These results show the ability of LAFA exemplary embodiments to detect enhanced PSGL-1 adhesive functions in patients with MS, but not IBD. Similar strategy may be used to detected altered adhesive functions of other adhesion molecules in other diseases.

### Example 30: Image and Data Analysis

The present example is directed to use to use Fiji image analysis software and R studio to process and analyse images generated during leukocyte adhesive function assay (LAFA) according to certain exemplary embodiment, so that a range of cell kinetic parameters may be determined and/or used to characterise the cell migratory behaviours. In addition, toi the example below other image software may be used to analyse the images and/or generate results.

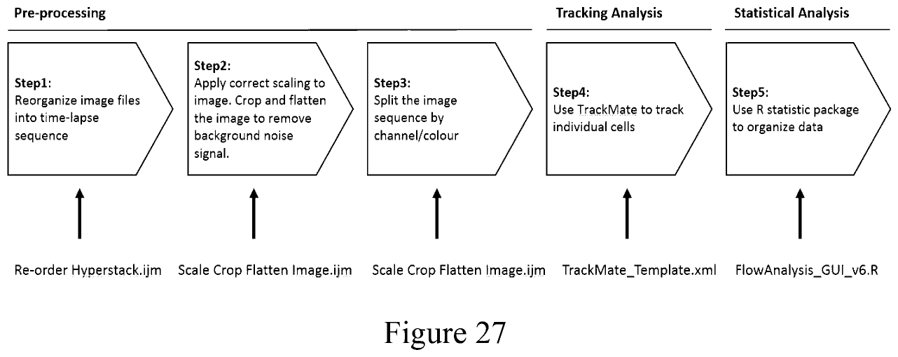

For example, as shown in FIG. 27 the images and data analysis process may consist of the following steps:
1. Raw TIF images captured with the microscope are opened in Fiji image analysis software and reorganized into a time-lapse sequence.
2. Correct scaling information is applied. Flow channel edges are removed from images by cropping. An image flattening algorithm is applied to remove uneven background fluorescence.
3. Image sequence is split into individual channels for analysis.
4. TrackMate plugin from Fiji software is used to track individual cells with a set cell size and intensity threshold per channel.
5. The outputs from TrackMate are further analysed by R Statistical Software package to convert the data to the desired measuring units for a range of cell kinetic parameters, including but not limited to cell numbers, speed, straightness, dwell time, diffusion coefficient.
6. and to generate descriptive statistical graphs (e. g. box-and-whisker plots of kinetic parameters, speed distribution histogram, straightness distribution histogram, duration histogram, dwell time distribution histogram, motility curves, common origin graphs, appearance graphs).

Further advantages of the claimed subject matter will become apparent from the following examples describing certain embodiments of the claimed subject matter.

Example 1A method to assess a subject's response, or potential response, to a drug treatment suitable for controlling progress of a disease, wherein the drug is capable of altering leukocyte recruitment, adhesion and/or migration, the method comprising the steps of: obtaining a blood sample from the subject:
subjecting the blood sample to at least one leukocyte function assay (LAFA), wherein the LAFA assesses leukocyte recruitment, adhesion and/or migration to at least one or more of the following: at least one endothelial molecule and at least one cell; and
based at least in part on one or more results of the at least one LAFA, assess a subject's respond, or potential response, to the drug treatment for controlling progression of the disease.

2A. A method to assess a subject's response, or potential response, to a drug treatment suitable for controlling progress of a disease, wherein the drug is capable of altering leukocyte recruitment, adhesion and/or migration, the method comprising the steps of:
obtaining a blood sample from the subject;
subjecting the blood sample to at least one leukocyte function assay (LAFA), wherein the at least one LAFA quantitatively and/or semi-quantitatively assesses leukocyte recruitment, adhesion and/or migration under realistic physiological conditions to at least one or more of the following: at least one endothelial molecule and at least one cell expressing an endothelial molecule; and
based at least in part on one or more results of the at least one LAFA. assess a subject's respond, or potential response, to the drug treatment for controlling progression of the disease.

3A. The method of example 1A, wherein the at least one LAFA is conducted on one or more of the following substrates: VCAM-1, MAdCAM-1, P-Selectin, E-selectin, IL-8. SDF1α and one or more cells expressing an endothelial molecule.

4A. The method of example 1A, wherein the at least one LAFA is conducted on two or more of the following substrates: VCAM-1, MAdCAM-1, P-Selectin, E-selectin, IL-8, SDF1α and one or more cells expressing an endothelial molecule.

5A. The method of example 1A, wherein the at least one LAFA is conducted on three or more of the following substrates: VCAM-1, MAdCAM-1, P-Selectin, E-selectin, IL-8, SDF1α and one or more cells expressing an endothelial molecule.

6A. The method of one or more of examples 1A-4A, wherein the at least one LAFA measures one or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

7A. The method of one or more of examples 1A-4A, wherein the at least one LAFA measures two or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

8A. The method of one or more of examples 1A-4A, wherein the at least one LAFA measures four or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

9A. The method of one or more of examples 1A-4A, wherein the at least one LAFA measures six or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocwte cells detected and an average dwell time of individual leukocyte cells detected.

10A. The method of one or more of examples 1A-9A, wherein the blood sample from the subject is treated with Mn2+ and the at least one LAFA is conducted on the Mn2+ treated blood sample and the one or more results of that at least one LAFA are used for generating one or more parameters that are used for generating one or more indexes.

11A. The method of one or more of examples 1A-10A, wherein at least one healthy blood sample is treated with Mn2+ and the at least one LAFA is conducted on the at least one healthy Mn2+ blood treated sample and the one or more results of the at least one LAFA are used for generating one or more parameters that are used for generating one or more indexes.

12A. The method of one or more examples 1A-11A, wherein the one or more results of the at least one LAFA from the blood sample from the subject is used as a control for generating one or more parameters that are used for generating one or more indexes.

13A. The method of one or more examples 1A-12A, wherein the one or more results of the at least one LAFA from at least one healthy blood sample is used as a control for generating one or more parameters that are used for generating one or more indexes.

14A. The method of one or more examples 1A-13A, wherein one or more of the following one or more indexes is generated: a relative straightness index (RSTI), a relative speed index (RSI) and a relative dwell time index (RDTI) for the subject.

15A. The method of one or more examples 1A-14A, wherein an activation potential ratio of the subject's blood is generated based on one or more results of the at least one LAFA from the blood of the subject divided by the one or more results of the at least one LAFA of Mn2+ treat blood sample of the subject.

16A. The method of one or more of examples 1A and 15A, wherein the disease at least in part involves one or more of the following: abnormal leukocyte recruitment, adhesion and/or migration; progression of inflammation; progression of an autoimmune state; progression of an immune deficiency state; and progression of an infectious state.

17A. The method of one or more of examples 1A and 16A, wherein the disease at least in part involves multiple sclerosis (MS).

18A. The method of one or more of examples 1A and 17A, wherein the disease at least in part involves of inflammatory bowel disease (IBD).

19A. The method of one or more of examples 1A-18A, wherein the one or more results of the at least one LAFA are used to stratify the subject and to assess a subject's respond, or potential response, to the drug treatment for controlling progression of the disease.

20A. The method of one or more of examples 1A-19A, wherein the one or more results of the at least one LAFA are used to stratify the subject and to predict a subject's respond, or potential response, to the drug treatment for controlling progression of the disease.

21A. The method of one or more examples 1A-20A, wherein the at least one LAFA is conducted under static or non-static conditions.

22A. A system for performing the at least one LAFA based on the methods of one or more examples 1A-21A.

23A. A device for performing the at least one LAFA based on the methods of one or more examples 1A-21A.

Example 1B. A method to assess adhesive function of one or more leukocytes molecules, the method comprising the steps of:
obtaining a blood sample from a subject;
subjecting the blood sample to at least one leukocyte function assay (LAFA), wherein the at least one LAFA quantitatively and/or semi-quantitatively assesses leukocyte recruitment, adhesion and/or migration under realistic physiological conditions to at least one or more of the following: at least one endothelial molecule and at least one cell expressing an endothelial molecule; and
based at least in part on one or more results of the at least one LAFA, assess a level of activation of the one or more leukocytes molecules.

2B. The method of example 1B, wherein the at least one LAFA is conducted on one or more of the following substrates: VCAM-1, MAdCAM-1, P-Selectin, E-selectin, IL-8. SDF1α and one or more cells expressing an endothelial molecule.

3B. The method of example 1B, wherein the at least one LAFA is conducted on two or more of the following substrates: VCAM-1, MAdCAM-1. P-Selectin, E-selectin, IL-8, SDF1α and one or more cells expressing an endothelial molecule.

4B. The method of example 1B, wherein the at least one LAFA is conducted on three or more of the following substrates: VCAM-1, MAdCAM-1, P-Selectin, E-selectin, IL-8, SDF1α and one or more cells expressing an endothelial molecule.

5B. The method of one or more of examples 1B-4B, wherein the at least one LAFA measures one or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

6B. The method of one or more of examples 1B-4B, wherein the at least one LAFA measures two or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

7B. The method of one or more of examples 1B-4B, wherein the at least one LAFA measures four or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

8B. The method of one or more of examples 1B-4B, wherein the at least one LAFA measures six or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

9B. The method of one or more of examples 1B-9B, wherein the blood sample from the subject is treated with Mn2+ and the at least one LAFA is conducted on the Mn2+ treated blood sample and the one or more results of that at least one LAFA are used for generating one or more parameters that are used for generating one or more indexes.

10B. The method of one or more of examples 1B-10B, wherein at least one healthy blood sample is treated with Mn2+ and the at least one LAFA is conducted on the at least one healthy Mn2+ blood treated sample and the one or more results of the at least one LAFA are used for generating one or more parameters that are used for generating one or more indexes.

11B. The method of one or more examples 1B-11B, wherein the one or more results of the at least one LAFA from the blood sample from the subject is used as a control for generating one or more parameters that are used for generating one or more indexes.

12B. The method of one or more examples 1B-12B, wherein the one or more results of the at least one LAFA from at least one healthy blood sample is used as a control for generating one or more parameters that are used for generating one or more indexes.

13B. The method of one or more examples 1B-13B, wherein one or more of the following one or more indexes is generated: a relative straightness index (RSTI), a relative speed index (RSI) and a relative dwell time index (RDTI) for the subject.

14B. The method of one or more examples 1B-14B, wherein an activation potential ratio of the subject's blood is generated based on one or more results of the at least one LAFA from the blood of the subject divided by the one or more results of the at least one LAFA of Mn2+ treat blood sample of the subject.

15B. The method of one or more of examples 1B and 15B, wherein the level of activation of the one or more leukocytes molecules is used to assess an adhesive function of the one or more leukocyte molecules in the blood sample from the subject with respect to a disease in the subject that at least in part involves one or more of the following: abnormal leukocyte recruitment, adhesion and/or migration; progression of inflammation; progression of an autoimmune state; progression of an immune deficiency state; and progression of an infectious state.

16B. The method of one or more of examples 1B and 16B, wherein the disease at least in part involves multiple sclerosis (MS).

17B. The method of one or more of examples 1B and 17B, wherein the disease at least in part involves of inflammatory bowel disease (IBD).

18B. The method of one or more of examples 1B-18B, wherein the one or more results of the at least one LAFA are used to stratify the subject and to assess a subject's respond, or potential response, to a drug treatment for controlling progression of the disease.

19B. The method of one or more of examples 1B-19B, wherein the one or more results of the at least one LAFA are used to stratify the subject and to predict a subject's respond, or potential response, to the drug treatment for controlling progression of the disease.

20B. The method of one or more examples 1B-20B, wherein the at least one LAFA is conducted under static or non-static conditions.

21B. The method of one or more examples 1B-20B, wherein the one or more results of the at least one LAFA are used to assess drug efficacy in the subject being treated with a drug.

22B. The method of one or more examples 1B-20B, wherein the one or more results of the at least one LAFA are used to assess drug sensitivity in the subject being treated with the drug.

23B. The method of one or more of examples 1B-20B, wherein the method is used to assess drug sensitivity in the subject, the method further comprising the steps of: (1) administering to the subject a known quantity of the drug for a predetermined period of time;
(2) after step (1), subjecting a further blood sample from the subject to at least one LAFA; and
(3) based on one or more results of the at least one LAFA. repeating steps (1) and (2) in order to assess drug sensitivity in the subject.

24B. The method of one or more of examples 1B-20B, wherein the method is used to assess drug sensitivity of the subject, the method further comprising the steps of:
(1) adding in vitro to one or more portions of the blood sample from the subject one or more known quantities of a drug;
(2) after step (1), subjecting the one or more portions of the blood sample to at least one LAFA; and
(3) based on one or more results of the at least one LAFA, determining drug sensitivity of the subject.

25B. The method of one or more of examples 1B-20B, wherein the method is used to monitor drug efficacy in the subject, the method further comprising the steps of:
(1) administering to the subject a known quantity of the drug for a predetermined period of time;
(2) after step (1), subjecting a further blood sample from the subject to at least one LAFA; and
(3) based on one or more results of the at least one LAFA, repeating steps (1) and (2) in order to monitor drug efficacy in the subject.

26B. The method of one or more of examples 1B-20B, wherein the method is used to monitor drug efficacy of the subject, the method further comprising the steps of:
(1) adding in vitro to one or more portions of the blood sample from the subject one or more known quantities of a drug;
(2) after step (1), subjecting the one or more portions of the blood sample to at least one LAFA; and
(3) based on one or more results of the at least one LAFA. determining drug efficacy of the subject.

27B. The method of one or more of examples 1B-20B, wherein the method is used to determine a minimum effective drug dose for the subject, the method further comprising the steps of:
(1) administering to the subject a known quantity of the drug for a predetermined period of time;
(2) after step (1), subjecting a further blood sample from the subject to at least one LAFA; and
(3) based on one or more results of the at least one LAFA, repeating steps (1) and (2) in order to determine the minimum effective drug dose in the subject.

28B. The method of one or more of examples 1B-20B, wherein the method is used to determine a minimum effective drug dose for the subject, the method further comprising the steps of:
(1) adding in vitro to the blood sample from the subject a known quantity of a drug;
(2) after step (1), subjecting the blood sample with the known quantity of the drug to at least one LAFA; and
(3) based on one or more results of the at least one LAFA, repeating steps (1) and (2) until a minimum effective drug dose for treating the subject is determined.

29B. The method of one or more of the examples 1B-27B, wherein the method reduces side effects in the subject being treated.

30B. The method of one or more of examples 1B-28B, wherein the method is used to determine the effect of a drug on the adhesive function of the one or more leukocyte molecules in the blood sample from the subject.

31B. The method of one or more of examples 1B-29B, wherein the method is used to identify disease markers of the subject.

32B. The method of one or more of examples 1B-29B, wherein the method is used to monitor the subject's health over a period of time, the method further comprising the steps of:
(1) obtaining one or more further blood samples from the subject and subjecting the one or more further blood samples to at least one LAFA; and
(2) repeating step (1) over a period of time in order to monitor the subject's health;
wherein the period of time between the one or more blood samples is at least one or more of the following: 1 week, 2 weeks, 3 weeks, 1 month, 2 months, 3 months, 4 months, 5 months, 6 months, 1 year, or 2 years.

A system for performing the at least one LAFA based on the methods of one or more examples 1B-32B.

A device for performing the at least one LAFA based on the methods of one or more examples 1B-32B.

Example 1C. A method of (a) predicting how a subject is likely to respond to a drug for controlling progression of a disease, (b) determining whether a drug can be used to control or prevent progression of a disease in a subject, (c) choosing a drug for preventing or controlling progression of a disease in a subject, or (d) identifying a drug for preventing or controlling progression of a disease in a subject wherein the drug is capable of altering leukocyte adhesion to an endothelial molecule, said method comprising the steps of:
subjecting at least one blood sample obtained from the subject to at least one leukocyte adhesive function assay in vitro; and
based on a result of the assay, (a) predicting how the at least one subject is likely to respond to the drug for controlling progression of the disease, (b) determining whether the drug can be used to control or prevent progression of the disease in the subject, (c) choosing a drug for preventing or controlling progression of the disease in the subject, or (d) identifying a drug for preventing or controlling progression of the disease in the subject.

2C. The method of example 1C, wherein the method is used for personalised medicine.

3C. The method of example 1C, wherein the method is used for distinguishing a drug responder from a drug non-responder.

4C. The method of example 1C, wherein the method is used for testing many subjects, for subject stratification (patient grouping).

5C. The method of example 1C, comprising the step of, in accordance with the assay result, trialing the drug on the subject for controlling progression of the disease.

6C. The method of example 1C, comprising the step of, in accordance with the assay result, treating the subject with the drug for controlling progression of the disease.

7C. The method of example 1C, comprising the step of, in accordance with the assay result, determining an effective minimum therapeutic dose of the drug for the subject for controlling progression of the disease whilst minimizing unwanted side effects caused by the drug.

8C. The method of example 7C, wherein the drug dose allows for restoration of minimal leukocyte cell interaction with endothelial cells in vivo so as to minimize or prevent pathologies such as progressive multifocal leukoencephalopathy (PML).

9C. The method of example 1C, comprising the step of, in accordance with the assay result, optimizing a dosage regimen for the drug for the subject for controlling progression of the disease, for example, by altering drug dosage or changing the length of time between sequential drug administrations.

10C. The method of example 1C, wherein the method is used for predicting or determining whether a drug (i.e. compound, chemical, molecule, reagent, biologic, antibody or other) could be useful for controlling progression of a disease for which the drug has not previously been indicated.

11C. The method of example 1C, wherein the assay of the method can comprise the step of identifying an adhesion anomaly or abnormality or a drug target and then choosing an appropriate drug for controlling progression of the disease based on the drug target.

12C. The method of example 1C, wherein the assay of the method can comprise the step of identifying an adhesion anomaly or abnormality or drug target and then choosing an appropriate drug for controlling progression of the disease based on a reference database of drug targets and drugs for those targets.

13C. The method of example 12C, wherein the method comprises the step of building a database of drug targets and drugs.

14C. The method of example 13C, wherein the database is built based on known drug targets and drugs.

15C. The method of example 13C, wherein the database is built based on in vivo drug treatments.

16C. The method of example 1C, wherein the assay of the method comprises the step of assaying for more than one adhesion anomaly or abnormality or drug target at the one time, preferably 2, 3, 4, 5, 6, 7, 8, 9 or 10 drug targets or more.

17C. The method of example 1C, wherein the method is a high throughput assay, testing for a plurality of adhesion anomalies or abnormalities or drug targets at the one time.

18C. The method of example 1C, wherein the method is used to generate a leukocyte adhesion fingerprint for a subject.

19C. The method of example 1C, wherein the method is used to identify different leukocyte anomalies or abnormalities in a subject.

20C. The method of example 1C, wherein the method is used for identifying disease markers.

21C. The method of example 1C, wherein the method is used to group individuals/subjects regardless of disease.

22C. The method of example 1C, further comprising the step of developing a treatment for the subject regardless of disease diagnosis.

23C. The method of example 1C, wherein the method is used for high throughput drug screening in vivo or in vitro.

24C. The method of example 1C, wherein the method is used for industry scale drug screening in laboratory animals.

25C. A method of determining how a subject administered a drug for controlling progression of a disease is responding to that drug, wherein the drug is capable of altering leukocyte adhesion to an endothelial molecule, said method comprising the steps of:
subjecting at least one blood sample containing the drug obtained from the subject to at least one leukocyte adhesive function assay in vitro; and
based on a result of the assay, determining how the subject is responding to the drug.

26C. The method of example 25C, wherein the method is used for personalized medicine.

27C. The method of example 25C, wherein the method is used for distinguishing a drug responder from a drug non-responder.

28C. The method of example 25C, wherein the method is used for testing many subjects, for subject stratification (patient grouping).

29C. The method of example 25C, wherein the method is used for high throughput drug.

Screening In Vivo or In Vitro.

30C. The method of example 25C, wherein the method is used for industry scale drug screening in laboratory animals.

31C. The method of example 25C, wherein the method comprises the step of, in accordance with the assay result, determining an effective minimum therapeutic dose of the drug for the subject for controlling progression of the disease whilst minimising unwanted side effects caused by the drug.

32C. The method of example 31C, wherein the drug dose allows for restoration of minimal leukocyte cell interaction with endothelial cells in vivo so as to minimise or prevent pathologies such as progressive multifocal leukoencephalopathy (PML).

33C. The method of example 25C, comprising the step of, in accordance with the assay result, optimising a dosage regimen for the drug for the subject for controlling progression of the disease, for example, by altering drug dosage or changing the length of time between sequential drug administrations.

34C. A method of optimising a dosage regimen for a subject taking a drug for controlling progression of a disease, wherein the drug is capable of altering leukocyte adhesion to an endothelial molecule, said method comprising the steps of: subjecting at least one blood sample containing the drug obtained from the subject to at least one leukocyte adhesive function assay in vitro; and based on a result of the assay, optimising the drug dosage regimen for the subject to control progression of the disease.

35C. The method of example 34C, comprising the step of, in accordance with the assay result, determining an effective minimum therapeutic dose of the drug for the subject for controlling progression of the disease whilst minimising unwanted side effects caused by the drug.

36C. The method of example 34C, wherein the drug dose allows for restoration of minimal leukocyte cell interaction with endothelial cells in vivo so as to minimise or prevent pathologies such as progressive multifocal leukoencephalopathy (PML).

37C. The method of example 34C, comprising the step of, in accordance with the assay result, optimising a dosage regimen for the drug for the subject for controlling progression of the disease, for example, by altering drug dosage or changing the length of time between sequential drug administrations.

38C. The method of example 34C, wherein each time the assay is carried out, the dosage regimen or minimum effective drug dose is optimised accordingly.

39C. The method of example 34C, wherein the method provides an accurate assessment of drug effectiveness, regardless of serum drug concentration.

40C. A method of determining a minimum effective drug dose for a subject for controlling progression of a disease, wherein the drug is capable of altering leukocyte adhesion to an endothelial molecule, said method comprising the steps of:
(1) administering to the subject a known amount of the drug for a predetermined period of time;
(2) after step (1), subjecting a blood sample containing the drug obtained from the subject to a leukocyte adhesive function assay in vitro; and
(3) based on a result of the assay, repeating steps (1) and (2) until a minimum effective drug dose for the subject can be determined for controlling progression of the disease.

41C. The method of example 40C, comprising minimizing unwanted side effects caused by the drug.

42C. The method of example 40C, comprising the step of, in accordance with the assay result, optimizing a dosage regimen for the drug for the subject for controlling progression of the disease.

43C. The method of example 40C, wherein drug sensitivity is tested in the subject using IC50 or IC99 to obtain the minimum effective drug dose.

44C. The method of example 40C, wherein the minimum effective drug dose allows for restoration of minimal leukocyte cell interaction with endothelial cells in vive so as to minimize or prevent pathologies such as progressive multifocal leukoencephalopathy (PML).

45C. The method of example 1C, wherein the method entails performing a leukocyte adhesive function assay to identify a leukocyte adhesion abnormality, choosing a suitable drug based on the nature of the leukocyte adhesion abnormality, and determining the effect of the drug on the leukocyte adhesion abnormality.

46C. The method of example 45C, comprising the following steps: 1. Subjecting a blood sample obtained from the subject to a leukocyte adhesive function assay to identify a leukocyte abnormality; 2. Choosing a suitable drug candidate (or more than one drug candidate) that could potentially be used for such an abnormality; 3. Treating a blood sample with the suitable drug candidate in vitro at various doses; 4. Performing a further leukocyte adhesive function assay to test the effect of the drug candidate on the leukocyte abnormality; 5. Choosing the best or most effective drug for the subject; 6. Administering the drug to the subject; 7. Taking blood from the subject at various time points post-drug administration; and, 8. Performing a leukocyte adhesive function assay to confirm the drug effect in the subject.

47C. The method of example 45C, wherein determining the effect of a drug in a human subject entails carrying out the following steps: I. Collecting blood from the subject; 2. Performing a first leukocyte adhesive function assay to obtain a baseline-3. Administering a drug to the subject; and, 4. Performing a leukocyte adhesive function assay at various time points postdrug administration to determine the drug effect.

48C. The method of example 45C, wherein determining the effect of a drug in an animal model/laboratory animal subject (mouse, primate etc) entails carrying out the following steps: 1. Collecting blood from the subject; 2. Performing a first leukocyte adhesive function assay to obtain a baseline; 3. Administering a drug to the subject at various doses (each dose will be an independent assay); and 4. Performing a leukocyte adhesive function assay at various time points post-drug administration to determine the drug effect.

49C. The method of example 45C, wherein determining the effect of a drug is carried out in vitro using the following steps: 1. Collecting blood from a subject; 2. Treating the blood with the drug at various doses; and, 3. Performing a leukocyte adhesive function assay at various time points after the start of drug treatment, to determine the drug effects and time required to reach such effects.

50C. The method of any one of the preceding examples, wherein the drug directly interferes with the binding of the leukocyte with the endothelial molecule.

51C. The method of any one of examples 1C-49C, wherein the drug indirectly interferes with the binding of the leukocyte with the endothelial molecule.

52C. The method of any one of examples 1C-49C, wherein the drug can target, bind to, associate with or otherwise interfere with a leukocyte adhesion molecule or other binding molecule of the leukocyte.

53C. The method of any one of examples 1C-49C, wherein the drug can target, bind to, associate with or otherwise interfere with the endothelial molecule.

54C. The method of any one of examples 1C-49C, wherein the drug can target, bind to, associate with or otherwise interfere with both a leukocyte adhesive molecule or other binding molecule and endothelial molecule.

55C. The method of any one of examples 1C-49C, wherein the drug can indirectly influence the adhesion/interaction between the leukocyte and endothelial molecule by way of exerting its effect upon another part or molecule of the leukocyte adhesion pathway.

56C. The method of any one of examples 1C-49C, wherein the drug can: regulate expression of a gene that affects leukocyte adhesion (for example the drug can act on intracellular signaling pathways to regulate the expression of a gene that affects leukocyte adhesion); affect posttranslational modification ofa gene product (RNA or protein) that affects leukocyte adhesion; regulate transportation or translocation of a gene product that affects leukocyte adhesion, and/or regulate the release from intracellular storage of a gene product that affects leukocyte adhesion.

57C. The method of any one of examples 1C-49C, wherein the leukocyte is a neutrophil, eosinophil, basophil, CD4 T lymphocyte, CD8 T lymphocyte, T regulatory cell, B lymphocytes, dendritic cell, monocyte or natural killer cell.

58C. The method of any one of examples 1C-49C, wherein the leukocyte adhesion molecule or other binding molecule of the leukocyte is a selectin, integrin, chemokine or chemokine receptor.

59C. The method of any one of examples 1C-49C, wherein the endothelial molecule is a selectin, cell adhesion molecule (CAM), chemokine or chemokine receptor.

60C. The method of any one of example 1C-49, wherein the leukocyte adhesion molecule is: PSGL-1, L-selectin, α1 integrin, α2 integrin, α3 integrin, α4 integrin, α5 integrin, α6 integrin, α7 integrin, α8 integrin, α9 integrin, α10 integrin, α11 integrin, αD integrin, αE integrin, αV integrin, αX integrin, CD11a (αL integrin), CD11b (αM integrin), β1 integrin, β2 integrin, β4 integrin, β5 integrin, β6 integrin, β7 integrin, β8 integrin, CD44, ESL-1, CD43, CD66, CD15 or ALCAM.

61C. The method of any one of examples 1C-49C, wherein the endothelial molecule is: E-selectin, Pselectin, VCAM-1, ICAM-1. ICAM-2, MadCAM-1, PECAM. GlyCAM-1, JAM-A, JAM-B, JAM-C, JAM-4, JAM-L, CD34, CD99, VAP-1, L-VAP-2, ESAM, E-LAM, cadherin, or hyaluronic acid.

62C The method of example 59C, wherein the chemokine and chemokine receptor are selected from the group consisting of: chemokine CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL26, CX3CL1, XCL1 and XCL2; chemokine receptor CXCR1, CXCR2, CXCR3, CXCR4, CXCR5, CXCR6, CCR1, CCR2, CCR3, CCR4, CCR5, CCR6, CCR7, CCR8, CCR9, CCR10, CCR11, CX3CR1 and XCR1.

63C. The method of any one of examples 1C-49C, wherein the drug can regulate the activity of a cytokine or chemokine.

64C. The method of any one of examples 1C-49C, wherein the drug can alter post-translational modification of an adhesion molecule or chemokine, alter protein membrane translocation of an adhesion molecule, regulate the release of an adhesion molecule from intracellular storage, act on intracellular signalling pathways to regulate the expression of an adhesion molecule or chemokine gene, or regulate mobilisation of an adhesion molecule.

65C. The method of any one of examples 1C-49C, wherein the drug attenuates leukocyte α4 integrin activation.

66C. The method of any one of examples 1C-49C, wherein the drug interferes with the interaction between leukocyte α4 integrin and its endothelial molecule.

67C. The method of any one of examples 1C-49C, wherein the drug interferes with the interaction between leukocyte-expressed PSGL-1 (P-selectin glycoprotein ligand-1) and its endothelial molecule, being P-selectin and/or E-selectin.

68C. The method of any one of examples 1C-49C, wherein the drug interferes with the interaction between leukocyte β2 integrin and its endothelial molecule.

69C. The method of any one of examples 1C-49C, wherein the drug interferes with the interaction between intercellular adhesion molecule-1 (ICAM-1) and/or vascular cell adhesion molecule-1 (VCAM-1) and its/their leukocyte adhesion molecule.

70C. The method of any one of examples 1C-49C, wherein the drug is an antibody that interferes with the binding between α4β7 integrin and MAdCAM-1.

71C. The method of example 70C, wherein the drug is Natalizumab.

72C. The method of any one of examples 1C-49C, wherein the drug is an antibody that interferes α4β7 integrin and MAdCAM-1.

73C. The method of example 72C, wherein the drug is Vedolizumab.

74C. The method of any one of examples 1C-49C, wherein the drug is an antibody that interferes CD11a (αL) and ICAM-1.

75. The method of example 74C, wherein the drug is Efalizumab or Odulimomab.

76C. The method of any one of examples 1C-49C, wherein the drug is an antibody that interferes with the binding between CD11b (αM) and ICAM-1.

77C. The method of example 76, wherein the drug is UK279, 276.

78C. The method of any one of examples 1C-49C, wherein the drug is an antibody that interferes with the binding between β2 integrin and its endothelial molecule.

79C. The method of example 78C, wherein the drug is Erlizumab or Roverlizumab.

80C. The method of any one of examples 1C-49C, wherein the drug is an antibody that interferes with the binding between β integrin and its endothelial molecule.

81C. The method of example 80C, wherein the drug is Etrolizumab.

82C. The method of any one of examples 1C-49C, wherein the drug is a steroid such as glucocorticoid (corticosteroid).

83. The method of example 82C, wherein the drug is a steroid such as Budesonide, Cortisone, Dexamethasone, Methylprednisolone, Prednisolone, or Prednisone.

84C. The method of any one of examples 1C-49C, wherein the drug is a non-steroidal anti-inflammatory drug (NSAID).

85C. The method of example 84C, wherein the drug is Celecoxib, Etoricoxib, Ibuprofen, Ketoprofen, Naproxen and Sulindac.

86C. The method of any one of examples 1C-49C, wherein the drug is an immune selective anti-inflammatory derivative (ImSAID).

87C. The method of example 86C, wherein the drug is Sub-mandibular gland peptide-T (SGP-T) or Phenylalanine-glutamine-glycine (FEG).

88C. The method of any one of examples 1C-49C, wherein the drug is a bioactive compound from a plant.

89C. The method of example 88C, wherein the drug is Plumbagin (from Plumbago zylanica) or Plumericin (from Himatanthus sucuuba).

90C. The method of any one of examples 1C-89C, wherein the drug is used to control a disease involving abnormal leukocyte recruitment.

91C. The method of any one of examples 1C-89C, wherein the drug is used to control a disease involving inflammation.

92C. The method of any one of examples 1C-89C, wherein the drug is used to control progression of an autoimmune disease.

93C. The method of any one of examples 1C-89C, wherein the drug is used to control progression of an immune-deficient disease.

94C. The method of any one of examples 1C-89C, wherein the drug is used to control progression of an infectious disease.

95C. The method of any one of examples 1C-89C, wherein the drug is used to control progression of multiple sclerosis, Crohn's disease, asthma, psoriasis or rheumatoid arthritis.

96C. The method of any one of examples 1C-89C, wherein the drug is used to control progression of disease caused by organ transplant, stroke, myocardial infarction or traumatic shock.

97C. The method of any one of the preceding examples, wherein the blood sample is whole blood.

98C. The method of any one of the preceding examples, wherein the leukocyte adhesive function assay/result is semi-quantitative and/or quantitative.

99C. The method of any one of the preceding examples, wherein the leukocyte adhesive function assay achieves one or more of the following: characterizing leukocyte cell recruitment; characterizing leukocyte cell tracking; characterizing leukocyte cell migratory behavior, in a quantitative manner.

100C. The method of any one of the preceding examples, wherein the leukocyte adhesive function assay entails quantitatively determining leukocyte migration.

101C. The method of example 100C, wherein leukocyte migration includes detecting, measuring or observing leukocyte cell tethering, rolling, slow rolling, firm adhesion, crawling and/or transendothelial migration.

102C. The method of any one of the preceding examples, wherein the leukocyte adhesive function assay entails detecting, measuring or observing leukocyte cell average speed, displacement, acceleration, deceleration, directionality, dwell time and/or straightness.

103C. The method of any one of the preceding examples, wherein the leukocyte adhesive function assay entails detecting, measuring or observing leukocyte migration under realistic physiological conditions.

104C. The method of any one of the preceding examples, wherein the assay allows for simultaneous detection of different leukocyte subsets.

105C. The method of any one of the preceding examples, wherein the leukocyte adhesive function assay involves a flow assay.

106C. The method of any one of the preceding examples, wherein the leukocyte adhesive function assay allows for visual analysis for characterizing leukocyte cell migratory behavior, characterizing leukocyte cell tracking, or characterizing leukocyte cell recruitment by the endothelial adhesion molecule.

107C. The method of any one of the preceding examples, wherein the endothelial molecule is in the form of a recombinant protein bound to a support or substrate.

108C. The method of any one of the preceding examples, wherein the endothelial molecule that can be used as adhesive substrate (i.e. bound to a support or substrate) in the leukocyte adhesive function assay is selected from the group consisting of: 1. An adhesion molecule; 2. Chemokine; 3. Purified antigen and artificial antigen-presenting cell system; 4. Other molecule that can regulate cell-cell interaction; and 5. Chemokine receptor.

109C. The method of any one of the preceding examples (in so far as they are relevant), wherein the leukocyte adhesive function assay entails detecting, measuring or observing the interaction between leukocyte-expressed PSGL-1 (P-selectin glycoprotein ligand-1) and its endothelial molecule, P-selectin and/or E-selectin.

110C. The method of any one of the preceding examples (in so far as they are relevant), wherein the LAFA entail quantitative assessment of a integrin adhesion function.

111C. The method of any one of the preceding examples (in so far as they are relevant), wherein the LAFA entails detecting, measuring or observing increased leukocyte α integrin expression and activity.

112C. The method of any one of the preceding examples (in so far as they are relevant), wherein the leukocyte adhesive function assay entails measuring, detecting or observing the interaction between leukocyte α4 integrin and endothelial VCAM-1.

113C. The method of any one of the preceding examples (in so far as they are relevant), wherein the leukocyte adhesive function assay entails detecting, measuring or observing the interaction between CD11a (αL integrin) and ICAM-1.

114C. The method of any one of the preceding examples (in so far as they are relevant), wherein the leukocyte adhesive function assay entails detecting, measuring or observing the interaction between CD11b (αM integrin) and ICAM-1.

115C. The method of any one of the preceding examples (in so far as they are relevant), wherein the leukocyte adhesive function assay entails detecting, measuring or observing the interaction between α4017 integrin and MAdCAM-1.

116C. The method of any one of the preceding examples (in so far as they are relevant), wherein the leukocyte adhesive function assay entails detecting, measuring or observing the interaction between intercellular adhesion molecule-1 (ICAM-1) and/or vascular cell adhesion molecule-1 (VCAM-1) and their leukocyte adhesion molecule.

117C. The method of any one of the preceding examples (in so far as they are relevant), wherein the leukocyte adhesive function assay entails detecting, measuring or observing the interaction between leukocyte β7 integrin and its endothelial molecule.

118C. The method of any one of the preceding examples (in so far as they are relevant), wherein the leukocyte adhesive function assay entails measuring one or more specific subsets of leukocytes, such as CD4, CD8 and CD15 cells.

119C. The method of any one of the preceding examples (in so far as they are relevant),

wherein the leukocyte adhesive function assay entails detecting, measuring or observing leukocyte migratory behaviours on cytokine or chemokine (e.g. THFα and IL-4) activated primary endothelial cell (e.g. HUVEC) or immobilised endothelial cell line (e.g. human microcirculation endothelial cell (HMEC)).

120C. The method of any one of the preceding examples (in so far as they are relevant), wherein the leukocyte adhesive function assay entails detecting, measuring or observing the effects of the drug Natalizumab on the leukocyte interaction with TNFα activated HUVEC.

121C. The method of any one of the preceding examples (in so far as they are relevant), wherein the leukocyte adhesive function assay entails detecting, measuring or observing Natalizumab-specific binding to α4 integrin on leukocytes.

122C. The method of any one of the preceding examples (in so far as they are relevant), wherein the leukocyte adhesive function assay entails measuring, detecting or observing the inhibitory effects of Natalizumab on the α4 integrin function.

123C. The method of any one of the preceding examples (in so far as they are relevant), wherein the leukocyte adhesive function assay entails simultaneously detecting, measuring or observing different leukocyte subsets by labelling the subsets with specific membrane markers.

124C. The method of any one of the preceding examples (in so far as they are relevant), wherein the method is carried out as a blood test, performed at various time points post-Natalizumab infusion, the assay results are used to determine the need of Natalizumab redosing, and the blood test is conducted in individual subjects to ensure drug effectiveness. facilitating the development of optimal/personalised treatment regimen for individual subjects.

125C. The method of any one of the preceding examples (in so far as they are relevant), wherein the method facilitates the restoration of immune response without comprising the drug efficacy, allowing enough immune response each dosing circle to effectively eliminate risk of PML.

126C. The method of example 1C, wherein when the leukocyte adhesive function assay is used for detecting activation of a drug target, the ability of the drug to control disease progression is predicted.

127C. The method of example 1C, wherein the method/assay is used to predict whether the drug can be used to control the progression of yet other diseases not known to be treatable using that drug.

128C. A method of generating a leukocyte adhesion profile for a subject, said method comprising the steps of:
subjecting at least one blood sample obtained from the subject to at least one leukocyte adhesive function assay in vitro so as to quantitatively assess the adhesion functions of different leukocyte subsets to one or more different endothelial molecules

at substantially the same time; and
using the assay result for: identifying leukocyte abnormalities; determination of personalised pathogenesis; identification of new disease markers for diseases; identifying early signs of disease: disease prediction; disease prevention; assisting with early an accurate diagnosis; developing an effective and personalised treatment for the subject; monitoring the health (healthy status) of the subject: grouping subjects regardless of disease; or developing a treatment for the subject regardless of disease diagnosis.

129C. The method of example 128C, when carried out on a blood sample obtained from a single subject.

130C. The method of example 128C, when carried out on blood samples obtained from a plurality of different subjects.

131C. The method of any one of examples 128C to 130C, wherein each subject is a healthy subject.

132. The method of example 128C or example 130C, wherein each subject has a disease.

133C. The method of any one of examples 128C to 132C, wherein the assay is carried out on a processed blood sample.

134C. The method of any one of examples 128C to 133C, wherein the leukocyte adhesive function assay is a flow (cell) assay for quantitating leukocyte cell migratory behaviour, leukocyte cell tracking, or leukocyte cell recruitment by the one or more endothelial adhesion molecules.

135C. The method of any one of examples 128C to 134C, wherein one or more endothelial molecules is/are in the form of a recombination protein bound to a support or substrate, or a cell system overexpressing the one or more endothelial adhesion molecules.

136C. The method of any one of examples 128C to 135C, wherein the leukocytes are as described in example 57.

137C. The method of any one of examples 128C to 135C, wherein leukocyte adhesion molecules or other binding molecules of the leukocyte are as described in examples 58C and 60.

138C. The method of any one of examples 128C to 135C, wherein one or more endothelial molecules are as described in examples 59 and 61C.

139C. The method of any one of examples 128C to 138C, when assaying the adhesion of different leukocyte subsets in individual flow channels, which can be pre-coated with specific endothelial molecule substrates, so that cell migration profiles for each adhesion molecule on a specific leukocyte subset can be generated.

140C. The method of any one of examples 128C to 139C, wherein the assay of the method comprises the step of identifying a drug target and then choosing an appropriate drug for controlling progression of the disease based the drug target.

141C. The method of any one of examples 128C to 140C, wherein the assay of the method comprises the step of identifying a drug target and then choosing an appropriate drug for controlling progression of the disease based on a reference database of drug targets and drugs for those targets.

142C. The method of example 141C, wherein the assay of the method comprises the step of building a database of drug targets and drugs.

143C. The method of any one of examples 128C to 142C, wherein the assay of the method comprises the step of assaying for more than one drug target at the one time (e.g. 2, 3, 4, 5, 6, 7, 8, 9 or 10 drug targets or more).

144C. The method of any one of examples 128C to 143C, wherein the method is a high throughput assay, testing for a plurality of drug targets at the one time.

### Example 1D

A method of (a) predicting how a subject is likely to respond to a drug for controlling progression of a disease. (b) determining whether a drug can be used to control or prevent progression of a disease in a subject, (c) choosing a drug for preventing or controlling progression of a disease in a subject, or (d) identifying a drug for preventing or controlling progression of a disease in a subject, wherein the drug is capable of altering leukocyte adhesion to an endothelial molecule, said method comprising the steps of:
subjecting at least one blood sample obtained from the subject to at least one leukocyte adhesive function assay in vitro; and
based on a result of the assay. (a) predicting how the at least one subject is likely to respond to the drug for controlling progression of the disease, (b) determining whether the drug can be used to control or prevent progression of the disease in the subject, (c) choosing a drug for preventing or controlling progression of the disease in the subject, or (d) identifying a drug for preventing or controlling progression of the disease in the subject.

2D. A method of determining how a subject administered a drug for controlling progression of a disease is responding to that drug, wherein the drug is capable of altering leukocyte adhesion to an endothelial molecule, said method comprising the steps of:
subjecting at least one blood sample containing the drug obtained from the subject to at least one leukocyte adhesive function assay in vitro; and
based on a result of the assay, determining how the subject is responding to the drug.

3D. A method of optimising a dosage regimen for a subject taking a drug for controlling progression of a disease, wherein the drug is capable of altering leukocyte adhesion to an endothelial molecule, said method comprising the steps of:
subjecting at least one blood sample containing the drug obtained from the subject to at least one leukocyte adhesive function assay in vitro; and
based on a result of the assay, optimising the drug dosage regimen for the subject to control progression of the disease.

4D. A method of determining a minimum effective drug dose for a subject for controlling progression of a disease, wherein the drug is capable of altering leukocyte adhesion to an endothelial molecule, said method comprising the steps of:
(1) administering to the subject a known quantity of the drug for a predetermined period of time;
(2) after step (1), subjecting a blood sample containing the drug obtained from the subject to a leukocyte adhesive function assay in vitro; and
(3) based on a result of the assay, repeating steps (1) and (2) until a minimum effective drug dose for the subject can be determined for controlling progression of the disease.

5D. A flow assay or flow device for carrying out the method as exampleed in any one of examples 1 to 4.

6D. A method of generating a leukocyte adhesion profile for a subject said method comprising the steps of:
subjecting at least one blood sample obtained from the subject to at least one leukocyte adhesive function assay in vitro so as to quantitatively assess the adhesion functions of different leukocyte subsets to one or more different endothelial molecules at substantially the same time; and
using the assay result for: identifying leukocyte abnormalities; determination of personalised pathogenesis; identification of new disease markers for diseases; identifying early signs of disease; disease prediction; disease prevention: assisting with early and accurate diagnosis; developing an effective and personalised treatment for the subject; monitoring the health (healthy status) of the subject; grouping different subjects regardless of disease; or developing a treatment for the subject regardless of disease diagnosis.

The term ‘comprise’ and variants of the term such as ‘comprises’ or ‘comprising’ are used herein to denote the inclusion of a stated integer or stated integers but not to exclude any other integer or any other integers, unless in the context or usage an exclusive interpretation of the term is required.

### APPENDIX A

### Protocol for Leukocyte Adhesive Functions Assay According to Certain Exemplary Embodiments (LAFA)

—VCAM-1 Assays

1. Introduction

The following protocol may be used with certain exemplary embodiments to carry out LAFA using VCAM-1 as substrate (VCAM-1 assays). Leukocyte adhesive function assay is an assay that may be used to assess the ability of leukocytes to interact with other molecules in blood, under certain flow conditions. Leukocytes are typically visualised in the blood by labelling with one or more fluorescent dyes, so that they may be detected by, for example, a fluorescent microscope. Suitable variations of this protocol are contemplated in some exemplary embodiments.

To mimic blood microcirculation in vitro, a microfluidic system may be used, which consist of a microfluidic pump and microfluidic chips/channels. Adhesive substrates may be pre-coated on the bottom of microfluidic channels, and blood samples may then be perfused through the channels, allowing leukocytes to interact with pre-coated adhesive substrates. These interactions may then be recorded by a microscope, and the images may then be analysed by suitable software. As a result, the cell interaction behaviours may be described using a range of cell kinetic parameters, from which the ability of leukocytes to interact with specific adhesive substrate may be assessed. The assessment may be qualitative, semi-substantially quantitative, quantitative or combinations thereof.

2. Reagents and Material
a) Human VCAM-1 protein (purchased from R&D system, Cat #: ADP5) After receiving the vial from R&D, VCAM-1 protein was reconstituted in HBSS buffer to a concentration of 1 mg/ml. The VCAM-1 solution was then aliquoted to 2 μl per tube (0.5 centrifuge tube), and stored at −80° C. freezer. When needed, one aliquot/tube of VCAM-1 is thawed out and used within 8 hours. No repeated freeze-thaw allowed.
b) Hanks' balanced salt solution (HBSS) (Sigma. Cat #: H1387) One package of HBSS powder is reconstituted into a 1 L water, and stored at 4° C. fridge.
c) Microfluidic chips: (Microfluidic ChipShop, Cat #: 01-0178-0152-01) PMMA, Lid thickness (175 μm) Straight channel chip (16 parallel channels), Mini Luer interface Width (1,000 μm)/Depth (200 μm)/Length (18 mm)
d) Chip Inlets
   i. Mini luer to luer adapter: can hold up to 70 μl blood
   ii. Mini luer to luer adapter plus 500 μl tank: can hold up to 500 μl blood
e) MnCl₂, (Sigma, Cat #: 450995) Make 0.5M stock, use 1:100 dilution in whole blood (5 mM of final concentration).
f) Fluorescence markers
   i. CD4-Alexa488 (BD. Cat #: 557695)
   ii. CD8-PE (BD, Cat #: 555635)
   iii. CD5-APC (BD, Cat #: 551376)
   iv. CD19-BV510 (BD, Cat #: 562947)
3. Microfluidic Chip Preparation
a. Thaw out the VCAM-1 protein from the −80° C. freezer, and dilute it to a concentration of 10 μg/ml with HBSS.
b. Use 151l of the diluted VCAM-1 protein solution to pre-coat each microfluidic channel, at 4° C. overnight.
c. Always leave the first channel on the chip empty for auto-focusing on the InCell.
d. Next day, channels need to be washed with HBSS once before being used for LAFA.

4. Blood Collection
a. 7-10 ml of whole blood via venepuncture is needed, which include 2 ml in EDTA tube (for FBE), and 5 ml in Lithium Heparin tube (for LAFA).
b. If a butterfly is used for blood collection, it is preferred to collect 2 ml blood in EDTA tube first, and then 5 ml in heparin tube. If a syringe is used, the order does not matter.
c. After collection, store blood tubes at room temperature (20° C.).
d. Avoid vigorously shaking to the blood tubes as it may activate the blood cells.

5. Blood Pre-Treatment and Labelling
a. For each assay, 130 μl heparinised blood is needed.
b. In some experiments, blood needs to be activated by 5 mM Mn for 5 min at room temperature (RT), before being used for the assay.
c. The following markers can be added alone or in any combination to the whole blood, incubating for 5 min at RT.
   CD4-Alexa488 (2 μl/100 μl whole blood)
   CD8-PE (1.5 μl/100 μl whole blood)
   CD15-APC (3 μl/100 μl whole blood)
   CD19-BV510 (2 μl/100l whole blood)
d. If testing drug effects (e.g. Natalizumab), the drug needs to be added to the blood for 10 min at RT before the assay. In Mn experiments, the drug needs to be added at least 5 min after the Mn treatment.

6. The Assay
a. Place the chip into the slide holder of the InCell 2200
b. Ture on both top and bottom heaters to 39° C. (which gives 35.5° C. to the slide)
c. Load the blood sample to the inlet of the chip
d. Connect the outlet of the chip to the microfluidic pump
e. Open up the protocol in InCell operating software
f. Find the focus plane
g. Start the pump/blood perfusion at 0.6 ml/hour
   10 ml syringe
   16G needle
h. Start the recording

7. Video Analysis
a. Open Fiji open-source image analysis software. Download and install the version suitable for your operating system prior to starting the analysis if you haven't used Fiji before (https://imagej.net/Fiji/Downloads).
b. Open macro ‘Re-order Hyperstack.ijm’ by dragging and dropping into Fiji.
c. Open macro ‘Scale Crop Flatten Image.ijm’ by dragging and dropping into Fiji.
d. Open B&C window (Image>Adjust>Brightness/Contrast).
e. Open TrackMate plugin (Plugins>Tracking>Load a TrackMate File) and select ‘TrackMate_Template.xml’.
f. Close window blank (V).
g. Open your images (Plugins>Bio-Formats>Bio-Formats Importer), double-click on the first image only, the rest will be loaded automatically.
h. Tick ‘Group files with similar names’, press ‘OK’. Everything else should be unticked, View should be ‘Hyperstack’, Color mode should be ‘Default’.
i. Tick ‘File name contains’ and type ‘A*.tif’ in the respective box.
j. Click on macro tab ‘Re-order Hyperstack.ijm’, press Run.
k. Click on macro tab ‘Scale Crop Flatten Image.ijm’, press Run.
l. In the B&C window, press ‘Auto’ to see the channel edges. Adjust ROI to include the channel center and exclude channel edges. Press ‘OK’.
m. Select the channel to be analysed (e.g. Dapi)
n. In the TrackMate window, press the green ‘left’ arrow button until greyed out. This is the start panel of the TrackMate analysis wizard.
o. Press ‘Refresh source’
p. Press green ‘right’ arrow button or ‘Please wait’ button twice.
q. Set Threshold to
   0.4 for Dapi
   1.0 for FITC
   3.0 for Cy3
   3.0 for Cy5
r. Press green ‘right’ arrow button or ‘Please wait’ button once, wait until detection is finished.
s. Press green ‘right’ arrow button or ‘Please wait’ button eight times to finish the remaining steps of the wizard.
t. Press ‘Analysis’ button, save the first table (Track statistics) as .csv file. Name pattern should include experiment number and channel (Example: Expt1_Dapi_Tracks.csv).
u. Discard the second table (Links in tracks statistics).
v. Save the third table (Spots in tracks statistics) as .csv file. Name pattern should include experiment number and channel (Example: Expt1_Dapi_Spots.csv).
w. Repeat steps m-v for all other channels to be analysed.
x. TrackMate .csv files can then be analysed in R.

### See FIG. 27

Re-order Hyperstack.ijm
Scale Crop Flatten Image.ijm
TrackMate_Template.xml

8. Secondary Data Analysis
a. OpenR
b. Open “FlowAnalysis_GUI_v6.R”, developed by StickyCell.
c. Go to the “FlowAnalysis” window
d. Choose “Full” from the “Analysis Type” menu
e. Click on “Browse Working Directory” to choose the fold where the files to be analysed are located.
f. Click on “Browse Control” to choose one Excel file, exported from “Trackmate”
g. Click on “Browse Patient” to choose another Excel file, exported from “Trackmate”
h. Press “Analyse” button.
i. All parameters will be generated in a sub-fold in the same fold.

### See FIG. 27

FlowAnalysis_GUI_v6.R

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A method to assess a subject's response, or potential response, to a drug treatment suitable for controlling progress of a disease, wherein the drug is capable of altering leukocyte recruitment, adhesion and/or migration, the method comprising the steps of:
obtaining a blood sample from the subject:
subjecting the blood sample to at least one leukocyte function assay (LAFA), wherein the LAFA assesses leukocyte recruitment, adhesion and/or migration to at least one or more of the following: at least one endothelial molecule and at least one cell; and
based at least in part on one or more results of the at least one LAFA, assess a subject's respond, or potential response, to the drug treatment for controlling progression of the disease.

2. A method to assess a subject's response, or potential response, to a drug treatment suitable for controlling progress of a disease, wherein the drug is capable of altering leukocyte recruitment, adhesion and/or migration, the method comprising the steps of:
obtaining a blood sample from the subject;
subjecting the blood sample to at least one leukocyte function assay (LAFA), wherein the at least one LAFA quantitatively and/or semi-quantitatively assesses leukocyte recruitment, adhesion and/or migration under realistic physiological conditions to at least one or more of the following: at least one endothelial molecule and at least one cell expressing an endothelial molecule; and
based at least in part on one or more results of the at least one LAFA, assess a subject's respond, or potential response, to the drug treatment for controlling progression of the disease.

3. The method of claim 1, wherein the at least one LAFA is conducted on one or more of the following substrates: VCAM-1, MAdCAM-1, P-Selectin, E-selectin, IL-8, SDF1α and one or more cells expressing an endothelial molecule.

4. The method of claim 1, wherein the at least one LAFA is conducted on two or more of the following substrates: VCAM-1, MAdCAM-1, P-Selectin, E-selectin, IL-8, SDF1α and one or more cells expressing an endothelial molecule.

5. The method of claim 1, wherein the at least one LAFA is conducted on three or more of the following substrates: VCAM-1, MAdCAM-1, P-Selectin, E-selectin, IL-8, SDF1α and one or more cells expressing an endothelial molecule.

6. The method of one or more of claims 1-4, wherein the at least one LAFA measures one or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

7. The method of one or more of claims 1-4, wherein the at least one LAFA measures two or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

8. The method of one or more of claims 1-4, wherein the at least one LAFA measures four or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

9. The method of one or more of claims 1-4, wherein the at least one LAFA measures six or more the following parameters: a quantification of rolling leukocyte cells detected, a quantification of adhesion leukocyte cells detected, a quantification of crawling leukocyte cells detected, an average speed of individual leukocyte cells detected, an average straightness of individual leukocyte cells detected, an average displacement of individual leukocyte cells detected and an average dwell time of individual leukocyte cells detected.

10. The method of one or more of claims 1-9, wherein the blood sample from the subject is treated with Mn2+ and the at least one LAFA is conducted on the Mn2+ treated blood sample and the one or more results of that at least one LAFA are used for generating one or more parameters that are used for generating one or more indexes.

11. The method of one or more of claims 1-10, wherein at least one healthy blood sample is treated with Mn2+ and the at least one LAFA is conducted on the at least one healthy Mn2+ blood treated sample and the one or more results of the at least one LAFA are used for generating one or more parameters that are used for generating one or more indexes.

12. The method of one or more claims 1-11, wherein the one or more results of the at least one LAFA from the blood sample from the subject is used as a control for generating one or more parameters that are used for generating one or more indexes.

13. The method of one or more claims 1-12, wherein the one or more results of the at least one LAFA from at least one healthy blood sample is used as a control for generating one or more parameters that are used for generating one or more indexes.

14. The method of one or more claims 1-13, wherein one or more of the following one or more indexes is generated: a relative straightness index (RSTI), a relative speed index (RSI) and a relative dwell time index (RDTI) for the subject.

15. The method of one or more claims 1-14, wherein an activation potential ratio of the subject's blood is generated based on one or more results of the at least one LAFA from the blood of the subject divided by the one or more results of the at least one LAFA of Mn2+ treat blood sample of the subject.

16. The method of one or more of claims 1 and 15, wherein the disease at least in part involves one or more of the following: abnormal leukocyte recruitment, adhesion and/or migration; progression of inflammation; progression of an autoimmune state; progression of an immune deficiency state; and progression of an infectious state.

17. The method of one or more of claims 1 and 16, wherein the disease at least in part involves multiple sclerosis (MS).

18. The method of one or more of claims 1 and 17, wherein the disease at least in part involves of inflammatory bowel disease (IBD).

19. The method of one or more of claims 1-18, wherein the one or more results of the at least one LAFA are used to stratify the subject and to assess a subject's respond, or potential response, to the drug treatment for controlling progression of the disease.

20. The method of one or more of claims 1-19, wherein the one or more results of the at least one LAFA are used to stratify the subject and to predict a subject's respond, or potential response, to the drug treatment for controlling progression of the disease.

21. The method of one or more claims 1-20, wherein the at least one LAFA is conducted under static or non-static conditions.

22. A system for performing the at least one LAFA based on the methods of one or more claims 1-21.

23. A device for performing the at least one LAFA based on the methods of one or more claims 1-21.
